(19) United States
(12) Patent Application Publication
(10) Pub. No.: US 2022/0298255 A1
(43) Pub. Date: Sep. 22, 2022

(54) E-CADHERIN ACTIVATING ANTIBODIES AND USES THEREOF

(71) Applicant: Seattle Children's Hospital d/b/a Seattle Children's Research Institute, Seattle, WA (US)

(72) Inventor: Barry M. Gumbiner, Seattle, WA (US)

(73) Assignee: Seattle Children's Hospital d/b/a Seattle Children's Research Institute, Seattle, WA (US)

(21) Appl. No.: 17/608,886

(22) PCT Filed: May 29, 2020
(86) PCT No.: PCT/US2020/035388

§ 371 Date: Nov. 4, 2021

Related U.S. Application Data
(60) Provisional application No. 62/855,525, filed on May 31, 2019.

(51) Int. Cl. C07K 16/28; A61P 35/04; A61P 29/00

(52) U.S. Cl. CPC C07K 16/2896; A61P 35/04; A61P 29/00; C07K 2317/24; A61K 2039/505

(57) ABSTRACT

Provided herein are several monoclonal antibodies that activate the adhesion activity of human and mouse E-cadherin, including the amino acid sequences for the CDRs that define the binding domains of each monoclonal antibody. Also described are methods of making these antibodies, as well as biologically functional fragments and derivatives thereof; and methods of using them in the treatment, prevention, and/or amelioration of disease and conditions characterized by disruption of normal cell adhesion and/or cell junctions. Specifically contemplated are methods and compositions for the treatment of cancer metastasis as well as inflammatory conditions (such as inflammatory bowel disease and airway inflammation).

### CROSS-REFERENCE TO RELATED APPLICATIONS

This is the U.S. National Phase of International Patent Application No. PCT/US2020/035388, filed May 29, 2020, which claims priority to and the benefit of the earlier filing date of U.S. Provisional Application No. 62/855,525, titled “E-CADHERIN ACTIVATING ANTIBODIES” and filed on May 31, 2019, each of which is incorporated by reference herein in its entirety.

### STATEMENT REGARDING FEDERALLY SPONSORED RESEARCH OR DEVELOPMENT

This invention was made with government support under grants GM122467 and CA207115 awarded by the National Institutes of Health. The government has certain rights in the invention.

### FIELD OF THE DISCLOSURE

The current disclosure provides engineered E-cadherin activating antibodies, as well as methods of their use, for instance in treating diseases such as cancer metastasis and inflammatory diseases/conditions.

### BACKGROUND OF THE DISCLOSURE

Traditional understanding of epithelial cancer metastasis is derived primarily from mouse models and it is thought to involve a series of sequential steps: Epithelial to mesenchymal transition (EMT) of individual cells within the primary tumor leading to their intravasation into the bloodstream, survival of such circulating tumor cells (CTCs) within the bloodstream, and finally their extravasation at distant sites, where mesenchymal-epithelial transition (MET) culminates in their proliferation as epithelial metastatic deposits. In light of its well-established function in maintaining adherens junctions, loss of E-cadherin epithelial cell adhesion protein ostensibly promotes metastasis by enabling the first step of the metastatic cascade: the disaggregation of cancer cells from one another. In addition, loss of the E-cadherin expression has been long considered to increase tumor cell invasiveness in vitro and contributes to the transition of adenoma to carcinoma in animal models. See, for instance, U.S. Pat. No. 7,569,668.

However, loss of E-cadherin expression is an oversimplification as many metastases still contain high levels of E-cadherin and epithelial cells expressing E-cadherin can become invasive and/or undergo an EMT-like process and metastasize in various cancers. Also, whether the loss of E-cadherin expression has successfully completed the various stages of the invasion-metastasis cascade is unclear. In fact, invasive leader cells in primary breast tumor and circulating tumor cell clusters in the blood still maintained expression of E-cadherin and E-cadherin is involved in collective cell behaviors that facilitate invasion and metastasis.

E-cadherin is a well-known important metastasis suppressor, but the regulation of its state of activation is almost unknown. Previous work on both Xenopus C-cadherin and human E-cadherin provided evidence for the regulation of cadherin adhesion activity independent of cell surface expression levels. Physiological regulation of C-cadherin in response to growth factors during embryonic morphogenesis involves changes in the adhesive state of cadherins at the cell surface, without changes in either expression levels at the cell surface or amounts of associated catenins. Furthermore, E-cadherin adhesive activity can be regulated at the cell surface by an inside-out signaling mechanism probably involving allosteric regulation of the homophilic adhesive bond, analogous to integrin regulation.

Additional information about cadherin, including E-cadherin, in regulating cell adhesion as well as involvement of E-cadherin in cancer, can be found in Petrova et al. (Mol. Biol. Cell. 23:2092-2108, 2012) and Petrova et al. (Mol. Biol. Cell, 27(21):3233-3244, 2016).

### SUMMARY OF THE DISCLOSURE

Specific monoclonal antibodies (mAbs) have been developed that can bind to E-cadherin and distinguish the activity and inactivity of E-cadherin from the cell surface. E-cadherin adhesive activity is dynamically regulated at the cell surface in tumor cells, and an activating monoclonal antibody to E-cadherin that induces a high adhesive state significantly decreased the number of cells metastasized to a distal organ without affecting the growth in size of primary tumor in the mammary gland. This indicates that low activity of E-cadherin on the surface of tumor cells is important for metastasis and that activation of its function with mAbs can suppress metastasis.

Amino acid sequences for monoclonal antibodies that activate the adhesion activity of human and mouse E-cadherin have been determined and are provided herein. They have been cloned into IgG1 and Fab cDNA backbones for expression and purification. These antibodies (including 19A11, 66E8, 56-4, and 18-5, as well as biologically active fragments and derivatives thereof) can be used in animal studies and for treatment of patients for various diseases, including cancer metastasis, inflammatory bowel disease, and inflammation of other epithelial organs, including the lung. In particular embodiments, activating E-cadherin antibodies include 56-4 and 18-5 that specifically bind to and activate mouse E-cadherin. Embodiments provide for use of antibodies that specifically bind to and activate mouse E-cadherin in experimental pre-clinical studies. In particular embodiments, activating E-cadherin antibodies include 19A11 and 66E8 that specifically bind to and activate human E-cadherin. Embodiments provide for use of antibodies that specifically bind to and activate human E-cadherin in treatment of subjects for diseases, including cancer metastasis, inflammatory bowel disease, and inflammation of other epithelial organs, including the lung.

Identified sequences and constructs allow for high expression and purification. Optionally, the provided sequences and constructs can be modified to increase the activity, targeting, and/or stability of the antibodies.

The antibodies, functional antibody fragments, and derivatives provided herein are useful for treating diseases of, for instance, epithelial tissues where there is disruption in normal cell adhesion or cell junctions. Such diseases include cancer metastasis in which cells become disorganized, as well as inflammatory diseases (including microbial infectious diseases, such as viral infections) where barrier function in mucosal tissues is disrupted due to loss of cell junctions.

The herein provided antibody molecules optionally may be targeted to desired tissue(s) for treatment; such targeting can help to limit off-target effects. Stability of mAbs or penetration can be overcome by engineering sequence changes.

The current disclosure provides an engineered (non-naturally occurring) antibody including: the heavy chain CDR1, CDR2 and CDR3 shown, respectively, in SEQ ID NO: 17, 18, and 19, and the light chain CDR1, CDR2 and CDR3 shown, respectively, in SEQ ID NO: 20, 21, and 22; or the heavy chain CDR1, CDR2 and CDR3 shown, respectively, in SEQ ID NO: 23, 24, and 25, and the light chain CDR1, CDR2 and CDR3 shown, respectively, in SEQ ID NO: 26, 27, and 28; or the heavy chain CDR1, CDR2 and CDR3 shown, respectively, in SEQ ID NO: 29, 30, and 31, and the light chain CDR1, CDR2 and CDR3 shown, respectively, in SEQ ID NO: 32, 33, and 34; or the heavy chain CDR1, CDR2 and CDR3 shown, respectively, in SEQ ID NO: 35, 36, and 37, and the light chain CDR1, CDR2 and CDR3 shown, respectively, in SEQ ID NO: 38, 39, and 40. In certain embodiments, the engineered antibody is a humanized antibody. By way of example, various of the claimed engineered antibodies may be any form of antibody or derivative thereof (which substantially maintaining binding to E-cadherin), including a Fab, an IgG, a scFv, a diabody, or bispecific antibody. It is specifically contemplated that examples of the engineered antibody binds specifically to and activates E-cadherin.

Also provided is an engineered antibody that binds specifically to and activates E-cadherin, which engineered antibody includes: the heavy chain variable domain shown in SEQ ID NO: 2 and the light chain variable domain shown in SEQ ID NO: 4; or the heavy chain variable domain shown in SEQ ID NO: 6 and the light chain variable domain having SEQ ID NO: 8; or the heavy chain variable domain shown in SEQ ID NO: 10 and the light chain variable domain shown in SEQ ID NO: 12; or the heavy chain variable domain shown in SEQ ID NO: 14 and the light chain variable domain shown in SEQ ID NO: 16. A specific engineered antibody includes the heavy chain variable domain shown in SEQ ID NO: 2 and the light chain variable domain shown in SEQ ID NO: 4. Another specific engineered antibody includes the heavy chain variable domain shown in SEQ ID NO: 6 and the light chain variable domain having SEQ ID NO: 8. Yet another specific engineered antibody includes the heavy chain variable domain shown in SEQ ID NO: 10 and the light chain variable domain shown in SEQ ID NO: 12. A fourth specific engineered antibody includes the heavy chain variable domain shown in SEQ ID NO: 14 and the light chain variable domain shown in SEQ ID NO: 16.

Specifically provided herein are engineered antibodies that include the monoclonal antibody 19A11, 66E8, as well as humanized versions and functional fragments thereof, that specifically bind to and activate human E-cadherin. The engineered antibody in some instances includes monoclonal antibody 56-4, 18-5, or a functional fragment thereof, that specifically binds to and activates mouse E-cadherin.

Also provided are polynucleotides encoding the described anti-E-cadherin antibodies. Examples of such polynucleotide include: (1) the polynucleotide sequence encoding the VH domain shown in SEQ ID NO: 1; or the polynucleotide sequence encoding the VL domain that is shown in SEQ ID NO: 3; or both; (2) the polynucleotide sequence encoding the VH domain shown in SEQ ID NO: 5; or the polynucleotide sequence encoding the VL domain that is shown in SEQ ID NO: 7; or both; (3) the polynucleotide sequence encoding the VH domain shown in SEQ ID NO: 9; or the polynucleotide sequence encoding the VL domain that is shown in SEQ ID NO: 11; or both; or (4) the polynucleotide sequence encoding the VH domain shown in SEQ ID NO: 13; or the polynucleotide sequence encoding the VL domain that is shown in SEQ ID NO: 15; or both.

Also provided are uses of the herein described anti-E-cadherin antibodies to treat, prevent, or ameliorate: cancer metastasis; inflammatory bowel disease; or airway inflammation; or use to treat, prevent, or ameliorate any disease or condition associated with or involving defective or disrupted epithelial barrier function. Another embodiment is a method for treating cancer in a subject, including: administering to a subject in need of such treatment a therapeutically effective amount of an activating E-cadherin engineered antibody provided herein or encoded by a polynucleotide described herein. The method of embodiment specifically includes instances where treating cancer includes reducing cancer metastasis.

Yet another embodiment is a method of treating a cancer patient with a cancer that expresses an E-cadherin protein, including: obtaining a tissue sample from an individual at risk of having a cancer that expresses an E-cadherin protein; determining the presence or absence or amount of the E-cadherin protein in the tissue sample in comparison to a control tissue sample from an individual known to be negative for the cancer; thereby diagnosing the cancer that expresses an E-cadherin protein, wherein the E-cadherin protein is expressed at normal or low levels, or is expressed by a subset of cells, or is overexpressed; and administering to the cancer patient with a cancer that expresses an E-cadherin protein an effective amount of the engineered antibody of any one of the provided embodiments or encoded by a polynucleotide of any of provided embodiments, or an antigen-binding antibody fragment thereof.

Yet another method embodiment is a method for treating a subject having an inflammatory disorder (such as inflammatory bowel disease or an airway inflammation), the method including: administering to a subject in need of such treatment a therapeutically effective amount of an engineered antibody of any one of disclosed embodiments or encoded by the disclosed polynucleotide. Specifically contemplated are methods that treat an inflammatory disorder including an autoimmune disease, as well as an inflammatory disorder characterized by disruption of normal cell adhesion and/or cell junctions.

Also contemplated are methods that treat infectious inflammatory diseases, such as lung infections that impact epithelial barrier function. Examples of such lung infections include bacterial and viral infections, including infections that induce acute respiratory distress syndrome (ARDS).

Also provided is a method for modulating cell adhesion of cadherin-expressing cells, which method includes: contacting the cells with an engineered anti-E-cadherin antibody of any one of the described embodiments or encoded by the polynucleotide of any of the described embodiments.

In examples of any of the provided method embodiments, the engineered antibody or encoding polynucleotide is administered locally to a site of inflammation or cancer in the subject.

In examples of any of the provided method embodiments, the engineered antibody binds (specifically) to and activates E-cadherin. By way of example, the engineered antibody in some instances includes monoclonal antibody 19A11, 66E8, as well as humanized versions and functional fragments thereof, that specifically bind to and activate human E-cadherin. The engineered antibody in some instances includes monoclonal antibody 56-4, 18-5, or a functional fragment thereof, that specifically binds to and activates mouse E-cadherin.

### BRIEF DESCRIPTION OF THE DRAWINGS

One or more of the drawings submitted herein are better understood in color, which is not available in patent application publications at the time of filing. Applicants consider the color versions of the drawings as part of the original submission and reserve the right to present color images of the drawings in later proceedings.

FIGS. 1A-1D. E-cadherin activation inhibits the tumor metastasis in the MMTV-PyMT mouse model of breast cancer. FIG. 1A, Schema of MMTV-PyMT breast cancer mouse model study. The MMTV-PyMT or FVB control female mice received intraperitoneal injections of either control neutral E-cadherin-specific mAb 19.1-10 or E-cadherin-activating mAb 56-4 twice weekly from 4 to 14 weeks of age. FIG. 1B, All palpable masses were measured weekly using external calipers until reaching a volume of 5 cm3. FIG. 1C, Bouin's fluid fixation (top) and H&E staining (middle) of the lung from 14-week-old MMTV-PyMT mice. Visible metastatic nodules after fixation were counted from individual animals and quantified. Scale bar, 500 μm. (FIG. 1B and FIG. 1C, n=14 to 16) ***, P<0.001, statistically significant compared with neutral antibody-treated control. FIG. 1D, Representative microscopic images of immunofluorescence staining for E-cadherin in lungs of MMTV-PyMT mice. Scale bar, 50 μm.

FIGS. 2A-2D. Recombinant activating mAbs to mouse E-cadherin. FIG. 2A. Activation assay using human colo205 cells expressing mouse E-cadherin (C18) versus colo205 cells in which E-cadherin has been knocked out using CRISPRCas9 (CR14). Shown are cells that were treated with 3 μg/mL of the noted recombinant mAb for 7 hrs.; this assay was also used for the initial hybridoma screen. FIG. 2B. Schematic of the structure of recombinant heavy and light chain proteins for all mAbs. Examples of all the recombinant antibodies contain the mouse IgG1 heavy chain Fc region and the Ig kappa light chain constant region. FIG. 2C. Western Blot analysis of the recombinant antibodies. Neutral (19.1-10) and activating (18-5, 56-4) antibodies at 1 μg/mL were blotted against 20 μg of whole cell lysate from mouse 4T1 cells. FIG. 2D. Immunofluorescence staining shows the mAbs bind to endogenous mouse E-cadherin as indicated, respectively. Parental 4T1 cells were incubated with neutral (19.1-10) and activating (18-5, 56-4) antibodies at 1 μg/mL for 7 hrs and then fixed with 4% paraformaldehyde (PFA).

FIGS. 3A-3D. New mouse E-cadherin activating antibody did not affect growth in size of primary tumor in MMTV-PyMT mouse model of breast cancer. FIG. 3A, 14-week-old MMTV-PyMT mice developed mammary tumors in similar size. FIG. 3B, Body weight was measured weekly. (n=14 to 16). FIGS. 3C, 3D. Representative microscopic images of immunofluorescence staining for E-cadherin (FIG. 3C) and injected antibody (FIG. 3D) in primary tumors or metastatic lungs of MMTV-PyMT mice. Scale bar, 50 μm. To identify the injected antibodies in tissues, paraffin sections were stained with secondary antibody Alexa Fluor 488 IgG1 alone without primary antibody staining (FIG. 3D).

FIGS. 4A-4D. Circulating tumor cells are reduced by E-cadherin activation in the breast cancer models. mRNA levels were analyzed by qRT-PCR and the estimated number of circulating tumor cells (CTCs) from equations for levels of mRNA expression in cultured Py2T or 4T1 cells was calculated. FIG. 4A, The MMTV-PyMT or FVB control female mice received intraperitoneal injections of either control neutral E-cadherin-specific mAb 19.1-10 or E-cadherin-activating mAb 56-4 twice weekly from 4 to 14 weeks of age. Circulating tumor cells in the peripheral blood were detected by mRNA levels of PyMT, E-cadherin, and EpCAM expression in MMTV-PyMT mouse model. (FIGS. 4A and 4B, FVB wild type n=3 and MMTV-PyMT n=5 to 7) *, P<0.05; **, P<0.01; ***, P<0.001, statistically significant compared with neutral antibody-treated control. FIGS. 4B-4D, Schema of 4T1 tumor cell grafted metastatic mouse model study. (FIG. 4B). Mouse epithelial 4T1 Luc2 cells expressing human E-cadherin (4T1 Luc2-hE) were injected into mammary fat pads of BALB/c mice. After 3 days, the mice were treated intraperitoneal injections of either control neutral E-cadherin-specific mAb 46H7 or E-cadherin-activating mAb 19A11 twice weekly until the end of the experiments. FIG. 4C, Number of pulmonary tumor nodules were examined by staining with Bouin's solution. (n=10) ***, P<0.001, statistically significant compared with neutral antibody-treated control. FIG. 4D, Circulating tumor cells in the peripheral blood were detected by mRNA levels of Luc2 and hE-cadherin expression in 4T1 Luc2-hE orthotopic grafted mouse model. (no graft group n=3 and tumor cell graft group n=8 to 9 **, P<0.01; ***, P<0.001, statistically significant compared with neutral antibody-treated control in tumor cell grafted group.

FIG. 5A-5B. E-cadherin activating antibody decreases tumor cell dissemination by inhibiting intravasation without affecting primary tumor growth. FIG. 5A. Representative microscopic images of H&E staining of the lung from 4T1 Luc2-hE cell injected mice. Scale bar, 500 μm. FIG. 5B, Tumors were measured weekly using external calipers.

FIGS. 6A-6C. E-cadherin activation delays the metastatic colonization of disseminated carcinoma cells. FIG. 6A, Schema of mouse model for metastatic colonization study by extravasation. Mouse epithelial 4T1 Luc2-hE cells were injected into tail-vein of BALB/c mice. One day after inoculation, the mice were treated intraperitoneal injections of either control neutral E-cadherin-specific mAb 46H7 or E-cadherin-activating mAb 19A11 twice weekly until the end of the experiments. FIG. 6B, Representative microscopic images of H&E stained sections (top). Representative image of lungs fixed with Bouin's (FIG. 6B, bottom) and quantification of visible metastatic tumor nodules (FIG. 6C). (n=15) Scale bar, 500 μm. **, P<0.01, statistically significant compared with neutral antibody-treated control group.

FIGS. 7A-7C. E-cadherin activation inhibits invasiveness of PyMT primary spheroids. FIG. 7A, Schema of MMTV-PyMT in 3D culture: (I) Isolation of primary mammary tumor; (II) Collagenase digestion and single cell suspension; (III) Spheroids formation in the lid of a tissue culture dish; (IV) Spheroid harvesting and plating with Matrigel/collagen mixture. The organoids were mixed to yield a suspension of 1-2 organoids/μl with a 3D extracellular matrix (growth factor-reduced Matrigel+Collagen I) and incubated for 5 days in the presence of mAbs. FIG. 7B, Representative image of organoid spheroids and enlarged views (FIG. 7B, bottom). Scale bar, 100 μm. FIG. 7C, Quantification of (FIG. 7B). Calculation of invasion as a function of the longest invasive distance emanating from the spheroid. Average of the longest invasive distance (μm) per spheroid. (n=30 organoids per group) ***, P<0.001, statistically significant compared with neutral antibody treatment.

FIGS. 8A-8D. E-cadherin activating antibody suppresses invasiveness in Py2T and 4T1 spheroids. Micrography of 3D collage-I-embedded organoids treated with neutral mAb or activating mAb. Representative microscopic images of Py2T spheroids (FIG. 8A) and quantification (FIG. 8B). Representative microscopic images of 4T1 spheroids (FIG. 8C) and quantification (FIG. 8D). Enlarged views of panels in FIGS. 8A and 8C, right of each image. Scale bar, 100 μm. Calculation of invasion as a function of the longest invasive distance emanating from the spheroid. Average of the longest invasive distance (μm) per spheroid. (n=50 organoids per group) ***, P<0.001, statistically significant compared with neutral antibody treatment.

FIGS. 9A-9G. E-cadherin activation inhibited cell adhesion, migration, and invasion in vitro. FIGS. 9A-9C, Py2T cell. FIGS. 9D-9F, 4T1 cell. For cell adhesion assay, activating mAbs and Fab fragments stimulated adhesion of cells to pure E-cadherin substrate. Py2T (FIG. 9A) or 4T1 (FIG. 9D) cells were untreated, pretreated with 3 μg/ml neutral mAb, 19.1-10 or activating mAb, 18.5 or 56-4 for 2 hr, and cell adhesion strength to E-cadherin-coated capillary tubes was evaluated using increasing laminar flow to determine the force required to detach cells. Migration (FIG. 9B and 9E) and invasion assay (FIG. 9C and 9F). The number of migrated cells in the in vitro migration assay of Py2T (FIG. 9B) or 4T1 (FIG. 9E) cells through a Boyden chamber and the number of invaded cells in the in vitro invasion assay of Py2T (FIG. 9C) or 4T1 (FIG. 9F) cells through a basement membrane. The cells were treated with of 3 μg/ml neutral mAb, 19.1-10 or activating mAb, 18.5 or 56-4 for 24 hr. The cells on the bottom part of the inserts were fixed and were subsequently stained with crystal violet. ***, P<0.001, statistically significant compared with neutral antibody treatment. FIG. 9G, Activating mAbs and Fab fragments (Neutral 19-1-10, Whole IgG 56-4, Fab 56-4) triggered compact epithelial morphology. 4T1 cells were treated with of 3 pg/ml neutral mAb, 19.1-10 or activating mAb, 56-4 for 24 hr. Data are representatives of one out of at least three independent experiments.

FIGS. 10A-10D. E-cadherin activating antibody inhibits cell migration and invasion in vitro. Transwell migration (FIGS. 10A and 10C) and invasion (FIGS. 10B and 10D) assays. Representative microscopic images of the experiments shown graphically in FIGS. 9B, 9C, 9E, and 9F (0.5% crystal violet stain, magnification ×200). In each panel, the six images are: No treatment, Neutral 19 1-10, Whole IgG 18.5, Whole IgG 65-4, Fab 18.5, and Fab 56.4.

FIGS. 11A-11D. E-cadherin activation increases cancer cell-specific apoptosis. Sections of lung from neutral or activating mAb-treated mice were examined for in situ apoptosis by TUNEL (FIG. 11A) or cleaved caspase-3 by immunofluorescence (FIG. 11B). Cells were measured in at least 10,000 cells and each percentage represents the average of three randomly chosen fields of 1 sample (×10) (n=4 per group). FIGS. 11C-11D, 4T1 and MCF10a cells were treated with neutral mAb 19.1-10 (4T1) and 46H7 (MCF10a) or activating mAb 56-4 (4T1) and 19A11 (MCF10a) for 24 hr. FIG. 11C, Immunofluorescence staining for cleaved caspase-3 was tested in the 4T1 and MCF10a cells. Each percentage represents the average of three randomly chosen fields of 1 sample (×10) (n=3 per group). FIG. 11D, Total RNA was prepared and analyzed for expression of the indicated transcripts by qRT-PCR using specific primers. ***, P<0.001, statistically significant compared with neutral antibody treatment. Data are representatives of one out of at least three independent experiments.

FIGS. 12A-12B. E-cadherin activating antibody enhanced apoptosis in metastatic lung. TUNEL assay (FIG. 12A) and Immunofluorescence staining for Cleaved-caspase-3 (FIG. 12B). Representative microscopic images of FIG. 12A and 12B. Scale bar, 50 μm. Hoechst staining is shown in the “merge” panels.

FIGS. 13A-13C. E-cadherin activation increase sensitivity to apoptosis in circulation. Mouse was treated intraperitoneal injections of either control neutral E-cadherin-specific mAb 46H7 or E-cadherin-activating mAb 19A11 twice weekly until the end of the experiments. One day after treatment, the mice were i.v. injected into mouse tail veins with 4T1 Luc2-hE cells and whole blood was collected at the indicated time point. CTCs expressing hE-cadherin were sorted by FACS. FIG. 13A, Percentage of CTCs expressing hE-cadherin in whole blood sample. FIGS. 13B-13C, mRNA expression of Bcl-xL (FIG. 13B) and Bax (FIG. 13C) in CTCs isolated by hE-cadherin. The levels were normalized by mRNA expression of hE-cadherin and the fold change was assessed with the control group treated with neutral mAb 46H7 at each time point. *, P<0.05; **, P<0.01; ***, P<0.001, statistically significant compared with neutral antibody treatment.

FIGS. 14A-14B. E-cadherin activating antibody reduces number of circulating tumor cells in the bloodstream. FIG. 14A, Schema of tumor cell graft animal experiment. Mouse was i.p. injected with either control neutral E-cadherin-specific mAb, 46H7 or E-cadherin-activating mAb, 19A11 twice weekly until the end of the experiments. One day after mAb treatment, the mice were i.v. injected into mouse tail veins with 4T1 Luc2-hE cells. Whole blood was collected at the indicated time point. FIG. 14B, mRNA level of Luc2 were analyzed by qRT-PCR and the estimated number of circulating tumor cells (CTCs) from equations for level of Luc2 mRNA expression in cultured 4T1Luc2-hE cells was calculated. **, P<0.01, statistically significant compared with neutral antibody treatment at each time point.

FIGS. 15A-15C. Cell proliferation was not altered by E-cadherin activating Ab. FIG. 15A, Quantification of immunofluorescence staining for pHistone3. The same sections as used in FIG. 12A and 12B were used. Representative microscopic images (FIG. 15A, bottom). Scale bar, 50 μm B, mRNA level of Ki67 were analyzed by qRT-PCR and normalized by Luc2 mRNA expression analyzed in the CTCs of FIG. 12. (FIG. 15B). FIG. 15C, Circulating tumor cells were sorted with hE-cadherin and then Ki67 mRNA level was analyzed by qRT-PCR. The RNA sample as used in FIG. 13 was tested.

FIG. 16. Importance of E-cadherin activation in metastatic cascade. The effects of E-cadherin activation in E-cadherin expressing tumor cells. The activation of E-cadherin (1) repress the cell migration and invasion as well as induce the cell adhesion, (2) can effectively inhibit metastasis to distant organs through the bloodstream by enhancement of the sensitivity of cancer cell-specific apoptosis.

FIG. 17. Activating mAbs to human E-cadherin inhibit the loss of human airway cell monolayer permeability caused by respiratory syncytial virus (RSV) infection. Human bronchial epithelial cell line 16HBE14O- (1.5×10⁵ cells from passage number between 12-16) were grown in transwell chambers (Costar catalogue #3470: 6.5 mm insert with 0.4 μm pore size) at liquid:liquid interface for one week to form a confluent monolayer. Cells were then either treated with activating monoclonal E-cad 19A11 Fab or with neutral monoclonal E-cad 46H7 Fab at 3 μg/ml concentration for 4 hours. 19A11 Fab treated or 46H7 Fab treated cells were then either left uninfected or infected with RSVL19 with MOI of 1 for 6, 24, and 48 hours. Trans epithelial electrical resistance (TEER) in each Fab treated 16HBE140-monolayer was measured before and after of RSVL19 infection (MOI 1) at indicated time points using STX chopstick electrode and EVOM (epithelial voltmeter instrument). TEER values were for blank filter was subtracted from each individual data point to determine the true tissue resistance for the monolayer. Unit area resistance was measured in triplicate and calculated for each condition and average values (ohm*cm²) were plotted in Y axis against time (hrs) at X axis. Error bars represent the average ±standard deviations from three independent transwells. MOI=multiplicity of infection.

FIGS. 18A-18B. Effect of E-Cadherin antibody in colonic length in mice. FIG. 18A. Age matched male and female (5 weeks old) IL10KO mice strain B6.129P2-II10tm1Cgn/J and corresponding control mice strain C57BL/6J animals were either fed standard lab based rodent diet or fed with NSAID (non-steroidal anti-inflammatory) group of drug Piroxicam (at a dose of 200 ppm=200 mg/kg) pelleted lab based rodent diet for 14 days. All four treatment groups were intraperitoneally injected either with activating monoclonal E-Cad antibody r56.4 or with control neutral antibody r19.1-10 for two weeks at a dose of 5 mg/kg twice weekly. At 7 weeks of age animals were euthanized followed by colonic length measurement in each individual mouse. The values for colon length in cm are plotted in Y axis for indicated experimental cohorts. Total number of mice participant in each cohort ranged between 4-5. FIG. 18B. Age matched male (6 weeks old) spontaneous ileitis mice strain SAMP1/YitFc and corresponding control mice strain AKR/J were intraperitoneally injected either with activating monoclonal E-Cad antibody r56.4 or with control neutral antibody r19.1-10 for 4 weeks at a dose of 5 mg/kg twice weekly. At 10 weeks of age animals were euthanized followed by colonic length measurement in each individual mouse. The values for colon length in cm are plotted in Y axis for indicated experimental cohorts. Total number of mice participant in each cohort ranged between 4-5.

FIGS. 19A-19C. Adoptive T cell transfer model of colitis in mice—activating mAbs for mouse E-cadherin reduce overt symptoms. Acute colitis was induced using a standard method by i.v. injection of a sorted subset of reactive T-cells, CD45Rb-high. CD45Rb-low T-cells were injected as a control that does not cause colitis. CD45Rb-high injected mice were subsequently treated by twice weekly IP injection of 5 mg/kg either E-cadherin activating mAb 56-4 or a control neutral mAb 19.1.10. FIG. 19A. Animals were monitored for weight loss. CD45Rb-high animals began to lose weight due to the onset of colitis, and this effect was reversed by activating mAb but not neutral mAb. FIG. 19B. Same data as in FIG. 19A but showing endpoints of weight loss when experiment was terminated. FIG. 19C. Colonic length, which is known to shorten in colitis. Colons were dissected from euthanized mice and measured.

FIGS. 20A-20C. E-Cadherin Activating mAb Reduced Pathology of Colitis, Adoptive T-cell transfer model. Histological analysis by HistoTox (Boulder, Colo.) of colons from animals described in FIGS. 19A-19C to examine pathology and extent of inflammation. Examples from individual mice are shown. All hematoxylin and eosin (H&E) stain. 100× magnification. FIG. 20A. Group 1 (CD45Rb-low, activating mAb 56.4 treated), Animal 217, Mouse Colon. Minimally affected colon is captured. Mucosal glands (M), submucosa (SM), tunica muscularis externa (TME), and a lymphoid aggregate (LA) are indicated. FIG. 20B. Group 2 (CD45Rb-high, activating mAb 56.4 treated), Animal 210, Mouse Colon. The mucosal glands (M) are minimally to mildly hyperplastic. Multifocally, inflammatory cells (neutrophils, macrophages, lymphocytes; arrows with asterisks) infiltrate the lamina propria of the mucosa and separate glands. Occasionally, regions of inflammation are associated with crypt infiltration and damage (arrows without asterisks). The submucosa (SM) and tunica muscularis externa (TME) are indicated. FIG. 20C. Group 3 (CD45Rb-high, neutral mAb 19.1-10 treated), Animal 219, Mouse Colon. The mucosal glands (M) are mildly to moderately hyperplastic. Inflammatory cells (macrophages, lymphocytes, and occasional neutrophils; arrows with asterisks) infiltrate the lamina propria of the mucosa and separate glands. Regions of inflammation are associated with crypt infiltration and damage (arrows without asterisks) resulting in some gland loss. The submucosa (SM) is minimally expanded by edema. The tunica muscularis externa (TME) is indicated.

FIGS. 21A-21D. E-Cadherin activating mAb for mouse E-cadherin reduced pathology of colitis -Adoptive T-cell transfer model. Pathology scoring by Histotox using their standard methods for assessing IBD. Pathology scoring of mice described in FIGS. 19A-19C analyzed histologically as shown by examples in FIGS. 20A-20C. Data combined for all animals in each cohort. FIG. 21A. Sum Colitis Scores. Standardized IBD scoring that includes parameters measured in FIGS. 21B-21D. FIG. 21B. Mean Edema Extent. FIG. 21C. Mean Histopathology Scores. Histological sections were scored for mucosal thickening (hyperplasia), degree of inflammation, gland damage, and erosion extent. FIG. 21D. Mean Neutrophil Score measures neutrophil invasion which increases in inflammation.

FIGS. 22A-22C. ID 10−/− (knockout) model of colitis—activating mAbs for mouse E-cadherin reduce overt symptoms. IL10 gene knockout mice develop colitis spontaneously, but more slowly over time. Mice were treated twice weekly with IP injection of 5 mg/kg of either E-cadherin activating mAb 56-4 or a control neutral mAb 19.1.10. FIG. 22A. Body weight over time. Animals did not lose weight during the short duration of this experiment, due a slower onset of colitis and continued normal weight gain due to growth. Nonetheless, 56.4 activating mAb supported greater weight gain compared to 19.1-10 neutral mAb, potentially due to slowing of colitis induced loss. FIG. 22B. Same data as in FIG. 22A but showing endpoints of body weight when experiment was terminated. FIG. 22C. Colonic length, which is known to shorten in colitis. Colons were dissected from euthanized mice and measured; colons were longer in activating mAb 56.4 treated mice.

FIGS. 23A-23B. Histological analysis of colons by HistoTox (Boulder, Colo.) from animals described in FIGS. 22A-22C to examine pathology and extent of inflammation. Examples from individual mice are shown. All H&E stain. 40× magnification. FIG. 23A. Group 1 (neutral mAb 19.1-10), Animal 201, Mouse Colon. The lamina propria of the mucosa (M) is infiltrated by low to moderate numbers of inflammatory cells (lymphocytes, macrophages, and few neutrophils; arrows with asterisks); inflammation mildly extends into the submucosa (SM). A region of the mucosal epithelium is hyperplastic and forms a polypoid-like structure extending into the lumen (arrows without asterisks mark the border); remaining epithelium is mildly hyperplastic or no hyperplasia. The tunica muscularis externa (TME) and mucosal lymphoid aggregates (LA) are indicated. FIG. 23B. Group 2 (activating mAb 56.4), Animal 175, Mouse Colon. Non-lesioned colon is captured. Mucosal glands (M), submucosa (SM), tunica muscularis externa (TME), and mucosal (mLA) and submucosal (smLA) lymphoid aggregates are indicated.

FIGS. 24A-24D. E-Cadherin activating mAb for mouse E-cadherin reduced pathology of colitis—IL10−/− (knockout) model of colitis. Pathology scoring by HistoTox (Boulder, Colo.) using their standard methods for assessing IBD. Pathology scoring of mice described in FIGS. 22A-22C analyzed histologically as shown by examples in FIGS. 23A, 23B. Data combined for all animals in each cohort. FIG. 24A. Sum Colitis Scores. Standardized IBD scoring that includes parameters measured in FIGS. 24B-24D. FIG. 24B. Mean Edema Extent. FIG. 24C. Mean Histopathology Scores. Histological sections were scored for mucosal thickening (hyperplasia), degree of inflammation, gland damage, and erosion extent. FIG. 24D. Mean Neutrophil Score measures neutrophil invasion which increases in inflammation.

FIGS. 25A, 25B. Atomic structures of fragments of two different activating mAbs to human E-cadherin bound to fragments of human E-cadherin, as determined by X-ray crystallography. Fabs are the antigen binding fragments of antibodies, produced by expression of cDNAs in mammalian cells. The E-cadherin fragment containing domains EC1-EC2 were produce by cDNA expression in bacteria. Complexes were formed, crystals produced, and imaged by X-ray crystallography. FIG. 25A. Fab from activating mAb 19A11 interacts with EC1 region. Structure deposited as PDB files in protein database found on World Wide Web at rcsb.org/structure/6CXY. FIG. 25B. Fab from activating mAb 66E8 interacts with EC2 and link between EC1 and EC2 region. Structure deposited as PDB files in protein database found on World Wide Web at rcsb.org/structure/6VEL.

### REFERENCE TO SEQUENCE LISTING

The nucleic acid sequences described herein are shown using standard letter abbreviations for nucleotide bases, as defined in 37 C.F.R. § 1.822. Only one strand of each nucleic acid sequence is shown, but the complementary strand is understood as included in embodiments where it would be appropriate. The Sequence Listing associated with this application is provided in text format in lieu of a paper copy, and is hereby incorporated by reference into the specification. The name of the text file containing the Sequence Listing is 2L24936.txt. The text file is 32 KB, was created on Nov. 3, 2021, and is being submitted electronically via EFS-Web.

SEQ ID NO: 1 is the heavy chain variable region DNA sequence of mouse anti-human E-cad monoclonal antibody mAB-1_19A11 (414 bp). Leader sequence (1-57)-FR1 (58-147)-CDR1 (148-162)-FR2 (163-204)-CDR2 (205-252)-FR3 (253-348)-CDR3 (349-381)-FR4 (381-414).

ATGGCTGTCCTGGGGCTGCTTCTCTGCCTGGTGACGTTCCCAAGCTGTGT
CCTGTCCCAGGTGCAGCTGAAGGAGTCAGGACCTGGCCTGGTGGCACCCT
CACAGAGCCTGTCCATCACATGCACGGTCTCTGGGTTCTCATTATCCAGA
TATGGTGTACACTGGGTTCGCCAGCCTCCAGGAAAGGGTCTGGAGTGGCT
GGGAATGATGTGGGGTGGTGGAAACACAGACTATAATTCAGCTCTCAAAT
CCAGACTGAGCATCAGCAAGGACAACTCCAAGAGCCAAGTTTTCTTAAAA
ATGAACAGTCTGCAAACTGATGACACAGCCATGTACTACTGTGCCAGTAG
TAACTACGTTCTTGGGTATGCTATGGACTACTGGGGTCAAGGAACCTCAG
TCACCGTCTCCTCA

SEQ ID NO: 2 is the heavy chain variable region amino acids sequence of mouse anti-human E-cad monoclonal antibody mAB-1_19A11 (138 aa). Leader sequence (1-19)-FR1 (20-49)-CDR1 (50-54)-FR2 (55-68)-CDR2 (69-84)-FR3 (85-116)-CDR3 (117-127)-FR4 (128-138).

MAVLGLLLCLVTFPSCVLSQVQLKESGPGLVAPSQSLSITCTVSGFSLSR
YGVHWVRQPPGKGLEWLGMMWGGGNTDYNSALKSRLSISKDNSKSQVFLK
MNSLQTDDTAMYYCASSNYVLGYAMDYWGQGTSVTVSS

SEQ ID NO: 3 is the light chain variable region DNA sequence of mouse anti-human E-cad monoclonal antibody mAB-1_19A11 (399 bp). Leader sequence (1-60)-FR1 (61-129)-CDR1 (130-180)-FR2 (181-225)-CDR2 (226-246)-FR3 (247-342)-CDR3 (343-369)-FR4 (370-399).

ATGGAATCACAGACCCAGGTCCTCATGTTTCTTCTGCTCTGGGTATCTGG
TGCCTGTGCAGACATTGTGATGACACAGTCTCCATCCTCCCTGGCTATGT
CAGTAGGACAGAAGGTCACTATGAACTGCAAGTCCAGTCAGAGTCTTTTA
AATAGTAGCAATCAAAAGAACTATTTGGCCTGGTACCAGCAGAAACCAGG
ACAGTCTCCTAAACTTCTGATATACTTTACATCCACTAGGGGATCTGGGG
TCCCTGATCGCTTCATAGGCAGTGGATCTGGGACAGATTTCACTCTTACC
ATCAGCAGTGTGGAGGCTGAAGACCTGGCAGATTACTTCTGTCAGCAACA
TTATAGAACTCCGCACACGTTCGGAGGGGGGACCAAGGTGGAAATAAAA

SEQ ID NO: 4 is the light chain variable region amino acid sequence of mouse anti-human E-cad monoclonal antibody mAB-1_19A11 (133 aa). Leader sequence (1-20)-FR1 (21-43)-CDR1 (44-60)-FR2 (61-75)-CDR2 (76-82)-FR3 (83-114)-CDR3 (115-123)-FR4 (124-133).

MESQTQVLMFLLLWVSGACADIVMTQSPSSLAMSVGQKVTMNCKSSQSLL
NSSNQKNYLAWYQQKPGQSPKLLIYFTSTRGSGVPDRFIGSGSGTDFTLT
ISSVEAEDLADYFCQQHYRTPHTFGGGTKVEIK

SEQ ID NO: 5 is the heavy chain variable region DNA sequence of mouse anti-human E-cad monoclonal antibody 66E8 (405 bp). Leader sequence (1-57)-FR1 (58-147)-CDR1 (148-162)-FR2 (163-204)-CDR2 (205-255)-FR3 (256-351)-CDR3 (351-372)-FR4 (373-405).

ATGGGATGGAGCTGTGTCTTTCTCTTTCTCCTGTCAGTAACTGTAGGTGT
GTTCTCTGAGGTTCAGCTGCAGCAGTCTGGACCTGAGCTGGTGAAGCCTG
GGGCTTCAGTGAAGATATCCTGCAAGGCTTCAGGTTACTCATTTACTGGC
TACTTTATGAACTGGGTGAAGCAGAGCCATGGAAAGAGCCTTGAGTGGAT
TGGACGTATTAATCCTTACAATGGTGATACTTTCTACAAGCAGAGGTTCA
AGGGCAAGGCCACATTGACTGTAGACAAATCCTCTAGCACAGTCCACATG
GACCTCCTGAGCCTGACATCTGAGGACTCTGCAGTCTATTATTGTGGAAG
AGGTAACTACTACTTTGACTACTGGGGCCAAGGCACCACTCTCACAGTCT
CCTCA

SEQ ID NO: 6 is the heavy chain variable region amino acid sequence of mouse anti-human E-cad monoclonal antibody 66E8 (135 aa). Leader sequence (1-19)-FR1 (20-49)-CDR1 (50-54)-FR2 (55-68)-CDR2 (69-85)-FR3 (86-117)-CDR3 (118-124)-FR4 (125-135).

MGWSCVFLFLLSVTVGVFSEVQLQQSGPELVKPGASVKISCKASGYSFTG
YFMNWVKQSHGKSLEWIGRINPYNGDTFYKQRFKGKATLTVDKSSSTVHM
DLLSLTSEDSAVYYCGRGNYYFDYWGQGTTLTVSS

SEQ ID NO: 7 is the light chain variable region DNA sequence of mouse anti-human E-cad monoclonal antibody 66E8 (381 bp). Leader sequence (1-60)-FR1 (61-129)-CDR1 (130-162)-FR2 (163-207)-CDR2 (208-228)-FR3 (229-264)-CDR3 (265-351)-FR4 (352-381).

ATGAGGTTCCAGGTTCAGGTTCTGGGGCTCCTTCTGCTCTGGATATCAGG
TGCCCAGTGTGATGTCCAGATAACCCAGTCTCCATCTTATCTTGCTGCAT
CTCCTGGAGAAACCATTACTATTAATTGCAGGACAAGTAAGAACATTAGC
AAGTATTTAGCCTGGTATCAAGAGAAACCTGGGAAAACTAATAAGCTTCT
TATCTACTCTGGATACACTTTGCAGTCTGGAATTCCATCAAGGTTCAGTG
GCAGTGGATCTGGTACAGATTTCACTCTCACCATCAGTAGCCTGGAGCCT
GAAGATTTTGCAATGTATTACTGTCAACAGCATAATGAATACCCGTACAC
GTTCGGAGGGGGGACCAAGCTGGAAATTAAA

SEQ ID NO: 8 is the light chain amino variable region acid sequence of mouse anti-human E-cad monoclonal antibody 66E8 (127 aa). Leader sequence (1-20)-FR1 (21-43)-CDR1 (44-54)-FR2 (55-69)-CDR2 (70-76)-FR3 (77-108)-CDR3 (109-117)-FR4 (118-127)

MRFQVQVLGLLLLWISGAQCDVQITQSPSYLAASPGETITINCRTSKNIS
KYLAWYQEKPGKTNKLLIYSGYTLQSGIPSRFSGSGSGTDFTLTISSLEP
EDFAMYYCQQHNEYPYTFGGGTKLEIK

SEQ ID NO: 9 is the heavy chain variable region DNA sequence of rabbit anti-mouse E-cad monoclonal antibody mAb-1_56-4 (426 bp). Leader sequence (1-60)-FR1 (61-147)-CDR1 (148-165)-FR2 (166-207)-CDR2 (208-261)-FR3 (262-354)-CDR3 (355-393)-FR4 (394-426)

ATGGAGACTGGGCTGCGCTGGCTTCTCCTGGTCGCTGTGCTCAAAGGTGT
CCAGTGTCAGCAGGAGCTGGAGGAGTCCGGGGGAGGCCTGGTCAGGCCTG
GGGCATCCCTGACACTCACCTGCAAAGCCTCTGGATTCGACCTCAGTAAC
TACTACTACTTGTGCTGGGTCCGCCAGTCTCCAGGGAAGGGGCTGGAGTG
GATCGCATGCATTTATGCTGGTGCTACTCATGACACTTACTACGCGAACT
GGGCGAAAGGCCGATTCACCATCTCCAGGACCTCGTCGACCACGGTGACT
CTGCAAATGACCAGTCTGACAGACGCGGACACGGCCACCTATTTCTGTGC
GAGAGACATTTTTGCTAGTGGTACTTATTATTATCGGGCCTTGTGGGGCC
CGGGCACCCTGGTCACCGTCTCCTCA

SEQ ID NO: 10 is the heavy chain amino acid sequence of rabbit anti-mouse E-cad monoclonal antibody mAb-1_56-4 (142 aa). Leader sequence (1-20)-FR1 (21-49)-CDR1 (50-56)-FR2 (57-69)-CDR2 (70-87)-FR3 (88-118)-CDR3 (119-131)-FR4 (132-142)

METGLRWLLLVAVLKGVQCQQELEESGGGLVRPGASLTLTCKASGFDLSN
YYYLCWVRQSPGKGLEWIACIYAGATHDTYYANWAKGRFTISRTSSTTVT
LQMTSLTDADTATYFCARDIFASGTYYYRALWGPGTLVTVSS

SEQ ID NO: 11 is the light chain variable region DNA sequence of rabbit anti-mouse E-cad monoclonal antibody mAb-1_56-4 (390 bp). Leader sequence (1-66)-FR1 (67-135)-CDR1 (136-168)-FR2 (169-213)-CDR2 (214-234)-FR3 (235-330)-CDR3 (331-360)-FR4 (361-390).

ATGGACATGAGGGCCCCCACTCAGCTGCTGGGGCTCCTGCTGCTCTGGCT
CCCAGGTGCCAGATGTGCATTCGAATTGACCCAGACTCCATCCTCCGTGG
AGGCAGCTGTGGGAGGCACAGTCACCATCAATTGCCAGGCCAGTCAGAGC
ATTAATAGTTGGTTATCCTGGTATCAGCAGAAACCAGGGCAGCCTCCCAA
GCTCCTGATCTATGCTGCATCCACTCTGGCATCTGGAGTCTCATCGCGGT
TCAAAGGCAGTGGATCTGGGACAGAGTTCGCTCTCACCATCAGCGACCTG
GAGTGTGCCGATGCTGCCACTTACTACTGTCAGAGCTATTATGGAACTAG
TACTACTGATTTCGGCGGAGGGACCGAGGTGGTGGTCAAA

SEQ ID NO: 12 is the light chain variable region amino acid sequence of rabbit anti-mouse E-cad monoclonal antibody mAb-1_56-4 (130 aa). Leader sequence (1-22)-FR1 (23-45)-CDR1 (46-56)-FR2 (57-71)-CDR2 (72-78)-FR3 (79-110)-CDR3 (111-120)-FR4 (121-130).

MDMRAPTQLLGLLLLWLPGARCAFELTQTPSSVEAAVGGTVTINCQASQS
INSWLSWYQQKPGQPPKLLIYAASTLASGVSSRFKGSGSGTEFALTISDL
ECADAATYYCQSYYGTSTTDFGGGTEVVVK

SEQ ID NO: 13 is the heavy chain variable region DNA sequence of rabbit anti-mouse E-cad monoclonal antibody mAb-2_18-5 (426 bp). Leader sequence (1-60)-FR1 (61-147)-CDR1 (148-165)-FR2 (166-207)-CDR2 (208-261)-FR3 (262-354)-CDR3 (355-393)-FR4 (34-426).

ATGGAGACTGGGCTGCGCTGGCTTCTCCTGGTCGCTGTGCTCAAAGGTGT
CCAGTGTCAGGAGCAGCTGGAGGAGTCCGGGGGAGGCCTGGTCAAGCCTG
GGGCATCCCTGACACTCACCTGCACAGCCTCTGGATTCGACCTCAGTACC
TATTTCTACATGTGCTGGGTCCGCCAGGCTCCAGGGAAGGGGCTGGAGTG
GATCGCATGCATTTATGTTGGTAGTACTGGTGACACTTACTACGCGAACT
GGGCGAAAGGCCGATTCACCATCTCCAAAACCTCGTCGACCACGGTGACT
CTGCAAATGACCAGTCTGACAGACGCGGACACGGCCACCTATTTCTGTGC
GAGAGACATTTTTGCTACTGGTATTAATTATTATCGGGCCTTGTGGGGCC
CAGGCACCCTGGTCACCGTCTCCTCA

SEQ ID NO: 14 is the heavy chain variable region amino acid sequence of rabbit anti-mouse E-cad monoclonal antibody mAb-2_18-5 (142 aa). Leader sequence (1-20)-FR1 (21-49)-CDR1 (50-55)-FR2 (56-69)-CDR2 (70-87)-FR3 (88-118)-CDR3 (119-131)-FR4 (132-142).

METGLRWLLLVAVLKGVQCQEQLEESGGGLVKPGASLTLTCTASGFDLST
YFYMCWVRQAPGKGLEWIACIYVGSTGDTYYANWAKGRFTISKTSSTTVT
LQMTSLTDADTATYFCARDIFATGINYYRALWGPGTLVTVSS

SEQ ID NO: 15 is the light chain variable region DNA sequence of rabbit anti-mouse E-cad monoclonal antibody mAb-2_18-5 (390 bp). Leader sequence (1-66)-FR1 (67-135)-CDR1 (136-168)-FR2 (169-213)-CDR2 (214-234)-FR3 (235-330)-CDR3 (331-360)-FR4 (361-390).

ATGGACATGAGGGCCCCCACTCAGCTGCTGGGGCTCCTGCTGCTCTGGCT
CCCAGGTGCCAGATGTGCATTCGAATTGACCCAGACTCCAGCCTCCGTGG
AGGCAGGTGTGGGAGGCACAGTCACCATCAAGTGCCAGGCCAGTGAGAGC
ATTAATAGTTGGTTATCCTGGTATCAGCAGAAACCAGGGCAGCCTCCCAC
GCTCCTGATCTATTCTGCATCCACTCTGGCATCTGGGGTCCCATCGCGTT
TCAAAGGCAGTAGATCTGGGACACAGTTCACTCTCACCATCAGCGACCTG
GAGTGTGCCGATGCTGCCACTTACTACTGTCAAAGCTATTATGGAACTAG
TACTACTGCTTTCGGCGGAGGGACCGAGGTGGTGGTCAAA

SEQ ID NO: 16 is the light chain variable region amino acid sequence of rabbit anti-mouse E-cad monoclonal antibody mAb-2_18-5 (130 aa). Leader sequence (1-22)-FR1 (23-45)-CDR1 (46-56)-FR2 (57-71)-CDR2 (72-78)-FR3 (79-110)-CDR3 (110-120)-FR4 (121-130).

MDMRAPTQLLGLLLLWLPGARCAFELTQTPASVEAGVGGTVTIKCQASES
INSWLSWYQQKPGQPPTLLIYSASTLASGVPSRFKGSRSGTQFTLTISDL
ECADAATYYCQSYYGTSTTAFGGGTEVVVK

SEQ ID NOs: 17-40 show the amino acid sequences of the complementarity determining regions (CDRs) for the provided monoclonal antibodies, as shown in the following table (Table 1). The first line for each provides the amino acid positions in the corresponding amino acid sequence of the heavy or light chain of each antibody:

TABLE 1

| | Heavy Chain | | | Light Chain | | |
|---|---|---|---|---|---|---|
| Antibody | CDR1 | CDR2 | CDR3 | CDR1 | CDR2 | CDR3 |
| 19A11 | 50-54 | 69-84 | 117-127 | 44-60 | 76-82 | 115-123 |
| | SEQ ID: 17 | SEQ ID: 18 | SEQ ID: 19 | SEQ ID: 20 | SEQ ID: 21 | SEQ ID: 22 |
| 66E8 | 50-54 | 69-85 | 118-124 | 44-54 | 70-76 | 109-117 |
| | SEQ ID: 23 | SEQ ID: 24 | SEQ ID: 25 | SEQ ID: 26 | SEQ ID: 27 | SEQ ID: 28 |
| 56-4 | 50-55 | 70-87 | 119-131 | 46-56 | 72-78 | 111-120 |
| | SEQ ID: 29 | SEQ ID: 30 | SEQ ID: 31 | SEQ ID: 32 | SEQ ID: 33 | SEQ ID: 34 |
| 18-5 | 50-55 | 70-87 | 119-131 | 46-56 | 72-78 | 111-120 |
| | SEQ ID: 35 | SEQ ID: 36 | SEQ ID: 37 | SEQ ID: 38 | SEQ ID: 39 | SEQ ID: 40 |

SEQ ID NOs: 41-60 show the nucleotide sequences of representative forward and reverse primers as follows:

TABLE 2

| | Forward primer | Reverse primer |
|---|---|---|
| hE-cadherin | CCCGCCTTATGATTCTCTGCTCGTGTCC | TCCGTACATGTCAGCCAGCTTCTTGAA |
| | SEQ ID NO: 41 | SEQ ID NO: 42 |
| hBcl-xL | TCCTTGTCTACGCTTTCCACG | GGTCGCATTGTGGCCTTT |
| | SEQ ID NO: 43 | SEQ ID NO: 44 |
| hBax | CCCGAGAGGTCTTTTTCCGAG | CCAGCCCATGATGGTTCTGAT |
| | SEQ ID NO: 45 | SEQ ID NO: 46 |
| hki67 | TGACCCTGATGAGAAAGCTCAA | CCCTGAGCAACACTGTCTTTT |
| | SEQ ID NO: 47 | SEQ ID NO: 48 |
| mE- | CGACCCTGCCTCTGAATCC | TACACGCTGGGAAACATGAGC |
| cadherin | SEQ ID NO: 49 | SEQ ID NO: 50 |
| mEpCAM | AGAATACTGTCATTTGCTCCAAACT | GTTCTGGATCGCCCCTTC |
| | SEQ ID NO: 51 | SEQ ID NO: 52 |
| mBcl-xL | TACCGGAGAGCGTTCAGTGA | CCATCCCGAAAGAGTTCATTCA |
| | SEQ ID NO: 53 | SEQ ID NO: 54 |
| mBax | TTGCTACAGGGTTTCATCCA | CATATTGCTGTCCAGTTCATCTC |
| | SEQ ID NO: 55 | SEQ ID NO: 56 |
| Luc2 | GCTCAGCAAGGAGGTAGGTG | TCTTACCGGTGTCCAAGTCC |
| | SEQ ID NO: 57 | SEQ ID NO: 58 |
| PyMT | CTCCAACAGATACACCCGCACATACT | GTATCCAGAAAGCGACCAAGACCAGC |
| | SEQ ID NO: 59 | SEQ ID NO: 60 |

SEQ ID NO: 61 is a representative mouse IgG heavy chain constant region DNA sequence.

GGGCAACCTAAGGCTCCATCAGTCTTCCCACTGGCCCCCTGCTGCGGGGA
CACACCCAGCTCCACGGTGACCCTGGGCTGCCTGGTCAAAGGCTACCTCC
CGGAGCCAGTGACCGTGACCTGGAACTCGGGCACCCTCACCAATGGGGTA
CGCACCTTCCCGTCCGTCCGGCAGTCCTCAGGCCTCTACTCGCTGAGCAG
CGTGGTGAGCGTGACCTCAAGCAGCCAGCCCGTCACCTGCAACGTGGCCC
ACCCAGCCACCAACACCAAAGTGGACAAGACCGTTGCGCCCTCGACATGC
AGCAAGCCCACGTGCCCACCCCCTGAACTCCTGGGGGGACCGTCTGTCTT
CATCTTCCCCCCAAAACCCAAGGACACCCTCATGATCTCACGCACCCCCG
AGGTCACATGCGTGGTGGTGGACGTGAGCCAGGATGACCCCGAGGTGCAG
TTCACATGGTACATAAACAACGAGCAGGTGCGCACCGCCCGGCCGCCGCT
ACGGGAGCAGCAGTTCAACAGCACGATCCGCGTGGTCAGCACCCTCCCCA
TCGCGCACCAGGACTGGCTGAGGGGCAAGGAGTTCAAGTGCAAAGTCCAC
AACAAGGCACTCCCGGCCCCCATCGAGAAAACCATCTCCAAAGCCAGAGG
GCAGCCCCTGGAGCCGAAGGTCTACACCATGGGCCCTCCCCGGGAGGAGC
TGAGCAGCAGGTCGGTCAGCCTGACCTGCATGATCAACGGCTTCTACCCT
TCCGACATCTCGGTGGAGTGGGAGAAGAACGGGAAGGCAGAGGACAACTA
CAAGACCACGCCGGCCGTGCTGGACAGCGACGGCTCCTACTTCCTCTACA
GCAAGCTCTCAGTGCCCACGAGTGAGTGGCAGCGGGGCGACGTCTTCACC
TGCTCCGTGATGCACGAGGCCTTGCACAACCACTACACGCAGAAGTCCAT
CTCCCGCTCTCCGGGTAAATGA

SEQ ID NO: 62 is a representative mouse IgG heavy chain constant region amino acid sequence.

GQPKAPSVFPLAPCCGDTPSSTVTLGCLVKGYLPEPVTVTWNSGTLTNGV
RTFPSVRQSSGLYSLSSVVSVTSSSQPVTCNVAHPATNTKVDKTVAPSTC
SKPTCPPPELLGGPSVFIFPPKPKDTLMISRTPEVTCVVVDVSQDDPEVQ
FTWYINNEQVRTARPPLREQQFNSTIRVVSTLPIAHQDWLRGKEFKCKVH
NKALPAPIEKTISKARGQPLEPKVYTMGPPREELSSRSVSLTCMINGFYP
SDISVEWEKNGKAEDNYKTTPAVLDSDGSYFLYSKLSVPTSEWQRGDVFT
CSVMHEALHNHYTQKSISRSPGK-

SEQ ID NO: 63 is a representative mouse IgG light chain constant region DNA sequence.

GGTGATCCAGTTGCACCTACTGTCCTCATCTTCCCACCAGCTGCTGATCA
GGTGGCAACTGGAACAGTCACCATCGTGTGTGTGGCGAATAAATACTTTC
CCGATGTCACCGTCACCTGGGAGGTGGATGGCACCACCCAAACAACTGGC
ATCGAGAACAGTAAAACACCGCAGAATTCTGCAGATTGTACCTACAACCT
CAGCAGCACTCTGACACTGACCAGCACACAGTACAACAGCCACAAAGAGT
ACACCTGCAAGGTGACCCAGGGCACGACCTCAGTCGTCCAGAGCTTCAAT
AGGGGTGACTGTTAG

SEQ ID NO: 64 is a representative mouse IgG light chain constant region amino acid sequence.

GDPVAPTVLIFPPAADQVATGTVTIVCVANKYFPDVTVTWEVDGTTQTTG
IENSKTPQNSADCTYNLSSTLTLTSTQYNSHKEYTCKVTQGTTSVVQSFN
RGDC-

### DETAILED DESCRIPTION

Provided herein are several monoclonal antibodies that activate the adhesion activity of human and mouse E-cadherin, including the amino acid sequences for the CDRs that define the binding domains of each monoclonal antibody. These binding domains have been cloned into IgG1 and Fab cDNA backbones, though other expression constructs and formats are contemplated. The E-cadherin activating antibodies (and biologically active fragments and derivatives thereof) can be expressed and purified, and will be useful in animal studies as well as for treating patients that have a disease or condition characterized by disruption of normal cell adhesion and/or cell junctions. Such diseases and conditions include cancer metastasis, inflammatory bowel disease, and inflammation of other epithelial organs. “E-cadherin” stands for epithelial-cadherin and is primarily expressed in epithelial tissues; it is frequently used as a marker for epithelial cells. There are some small exceptions; for instance, E-cadherin has been observed in a small number of neurons/synapses despite the fact that N-cadherin is the predominant cadherin in neural tissue. In general E-cadherin is not expressed in mesenchymal, endothelial, muscle cells, etc.

Thus, there is provided herein an engineered antibody including: the heavy chain CDR1, CDR2 and CDR3 shown, respectively, in SEQ ID NO: 17, 18, and 19, and the light chain CDR1, CDR2 and CDR3 shown, respectively, in SEQ ID NO: 20, 21, and 22; or the heavy chain CDR1, CDR2 and CDR3 shown, respectively, in SEQ ID NO: 23, 24, and 25, and the light chain CDR1, CDR2 and CDR3 shown, respectively, in SEQ ID NO: 26, 27, and 28; or the heavy chain CDR1, CDR2 and CDR3 shown, respectively, in SEQ ID NO: 29, 30, and 31, and the light chain CDR1, CDR2 and CDR3 shown, respectively, in SEQ ID NO: 32, 33, and 34; or the heavy chain CDR1, CDR2 and CDR3 shown, respectively, in SEQ ID NO: 35, 36, and 37, and the light chain CDR1, CDR2 and CDR3 shown, respectively, in SEQ ID NO: 38, 39, and 40. In certain embodiments, the engineered antibody is a humanized antibody. By way of example, various of the claimed engineered antibodies may be any form of antibody or derivative thereof (which substantially maintaining binding to E-cadherin), including a Fab, an IgG, a scFv, a diabody, or bispecific antibody.

It is specifically contemplated that examples of the engineered antibody binds specifically to and activates E-cadherin.

Also provided is an engineered antibody that binds specifically to and activates E-cadherin, which engineered antibody includes: the heavy chain variable domain shown in SEQ ID NO: 2 and the light chain variable domain shown in SEQ ID NO: 4; or the heavy chain variable domain shown in SEQ ID NO: 6 and the light chain variable domain having SEQ ID NO: 8; or the heavy chain variable domain shown in SEQ ID NO: 10 and the light chain variable domain shown in SEQ ID NO: 12; or the heavy chain variable domain shown in SEQ ID NO: 14 and the light chain variable domain shown in SEQ ID NO: 16. A specific engineered antibody includes the heavy chain variable domain shown in SEQ ID NO: 2 and the light chain variable domain shown in SEQ ID NO: 4. Another specific engineered antibody includes the heavy chain variable domain shown in SEQ ID NO: 6 and the light chain variable domain having SEQ ID NO: 8. Yet another specific engineered antibody includes the heavy chain variable domain shown in SEQ ID NO: 10 and the light chain variable domain shown in SEQ ID NO: 12. A fourth specific engineered antibody includes the heavy chain variable domain shown in SEQ ID NO: 14 and the light chain variable domain shown in SEQ ID NO: 16.

Specifically provided herein are engineered antibodies that include the monoclonal antibody 19A11, 66E8, as well as humanized versions and functional fragments thereof, that specifically bind to and activate human E-cadherin. The engineered antibody in some instances includes monoclonal antibody 56-4, 18-5, or a functional fragment thereof, that specifically binds to and activates mouse E-cadherin.

Also provided are polynucleotides encoding the described anti-E-cadherin antibodies. Examples of such polynucleotide include: (1) the polynucleotide sequence encoding the VH domain shown in SEQ ID NO: 1; or the polynucleotide sequence encoding the VL domain that is shown in SEQ ID NO: 3; or both; (2) the polynucleotide sequence encoding the VH domain shown in SEQ ID NO: 5; or the polynucleotide sequence encoding the VL domain that is shown in SEQ ID NO: 7; or both; (3) the polynucleotide sequence encoding the VH domain shown in SEQ ID NO: 9; or the polynucleotide sequence encoding the VL domain that is shown in SEQ ID NO: 11; or both; or (4) the polynucleotide sequence encoding the VH domain shown in SEQ ID NO: 13; or the polynucleotide sequence encoding the VL domain that is shown in SEQ ID NO: 15; or both.

Also provided are uses of the herein described anti-E-cadherin antibodies to treat, prevent, or ameliorate: cancer metastasis; inflammatory bowel disease; or airway inflammation; or use to treat, prevent, or ameliorate any disease or condition associated with or involving defective or disrupted epithelial barrier function.

Another embodiment is a method for treating cancer in a subject, including: administering to a subject in need of such treatment a therapeutically effective amount of an activating E-cadherin engineered antibody provided herein or encoded by a polynucleotide described herein. The method of embodiment specifically includes instances where treating cancer includes reducing cancer metastasis.

Yet another embodiment is a method of treating a cancer patient with a cancer that expresses an E-cadherin protein, including: obtaining a tissue sample from an individual at risk of having a cancer that expresses an E-cadherin protein; determining the presence or absence or amount of the E-cadherin protein in the tissue sample in comparison to a control tissue sample from an individual known to be negative for the cancer; thereby diagnosing the cancer that expresses an E-cadherin protein, wherein the E-cadherin protein is expressed at normal or low levels, or is expressed by a subset of cells, or is overexpressed; and administering to the cancer patient with a cancer that expresses an E-cadherin protein an effective amount of the engineered antibody of any one of the provided embodiments or encoded by a polynucleotide of any of provided embodiments, or an antigen-binding antibody fragment thereof.

Yet another method embodiment is a method for treating a subject having an inflammatory disorder (such as inflammatory bowel disease or an airway inflammation), the method including: administering to a subject in need of such treatment a therapeutically effective amount of an engineered antibody of any one of disclosed embodiments or encoded by the disclosed polynucleotide. Specifically contemplated are methods that treat an inflammatory disorder including an autoimmune disease, as well as an inflammatory disorder characterized by disruption of normal cell adhesion and/or cell junctions.

In examples of any of the provided method embodiments, the engineered antibody or encoding polynucleotide is administered locally to a site of inflammation or cancer in the subject.

In examples of any of the provided method embodiments, the engineered antibody binds (specifically) to and activates E-cadherin. By way of example, the engineered antibody in some instances includes monoclonal antibody 19A11, 66E8, as well as humanized versions and functional fragments thereof, that specifically bind to and activate human E-cadherin. The engineered antibody in some instances includes monoclonal antibody 56-4, 18-5, or a functional fragment thereof, that specifically binds to and activates mouse E-cadherin.

Also provided is a method for modulating cell adhesion of cadherin-expressing cells including: contacting the cells with an engineered anti-E-cadherin antibody of any one of the described embodiments or encoded by the polynucleotide of any of the described embodiments.

Aspects of the disclosure are now described with additional detail and options to support the teachings of the disclosure, as follows: (I) Activating E-Cadherin Antibodies; (II) Production of Antibodies and Antibodies Variations; (III) Pharmaceutical Compositions and Formulations; (IV) Exemplary Methods of Use; (V) Kits; and (VI) Exemplary Embodiments.

(I) Activating E-Cadherin Antibodies

Provided herein are several E-cadherin activating monoclonal antibodies. These include specifically mouse anti-human E-cadherin monoclonal antibody mAB-1_19A11 (Petrova et al., Mol. Biol. Cell. 23:2092-2108, 2012), mouse anti-human E-cadherin monoclonal antibody 66E8; rabbit anti-mouse E-cadherin monoclonal antibody mAb-1_56-4; and rabbit anti-mouse E-cadherin monoclonal antibody mAb-2_18-5.

The following table (Table 3) provides the nucleotide (odd SEQ ID NOs) and amino acid (even SEQ ID NOs) positions corresponding to specific regions (leader, framework, complementarity determining) in the heavy and light chains of the variable domains of four representative activating E-cadherin antibodies:

TABLE 3

| | SEQ | | | | | | | | |
|---|---|---|---|---|---|---|---|---|---|
| mAb | ID: | Leader | FR1 | CDR1 | FR2 | CDR2 | FR3 | CDR3 | FR4 |
| 19A11 | 1 | 1-57 | 58-147 | 148-162 | 163-204 | 205-252 | 253-348 | 349-381 | 381-414 |
| | 2 | 1-19 | 20-49 | 50-54 | 55-68 | 69-84 | 85-116 | 117-127 | 128-138 |
| | 3 | 1-60 | 61-129 | 130-180 | 181-225 | 226-246 | 247-342 | 343-369 | 370-399 |
| | 4 | 1-20 | 21-43 | 44-60 | 61-75 | 76-82 | 83-114 | 115-123 | 124-133 |
| 66E8 | 5 | 1-57 | 58-147 | 148-162 | 163-204 | 205-255 | 256-351 | 352-372 | 373-405 |
| | 6 | 1-19 | 20-49 | 50-54 | 55-68 | 69-85 | 86-117 | 118-124 | 125-135 |
| | 7 | 1-60 | 61-129 | 130-162 | 163-207 | 208-228 | 229-264 | 265-351 | 352-381 |
| | 8 | 1-20 | 21-43 | 44-54 | 55-69 | 70-76 | 77-108 | 109-117 | 118-127 |
| 56-4 | 9 | 1-60 | 61-147 | 148-165 | 166-207 | 208-261 | 262-354 | 355-393 | 394-426 |
| | 10 | 1-20 | 21-49 | 50-56 | 57-69 | 70-87 | 88-118 | 119-131 | 132-142 |
| | 11 | 1-66 | 67-135 | 136-168 | 169-213 | 214-234 | 235-330 | 331-360 | 361-390 |
| | 12 | 1-22 | 23-45 | 46-56 | 57-71 | 72-78 | 79-110 | 111-120 | 121-130 |
| 18-5 | 13 | 1-60 | 61-147 | 148-165 | 166-207 | 208-261 | 262-354 | 355-393 | 394-426 |
| | 14 | 1-20 | 21-49 | 50-55 | 56-69 | 70-87 | 88-118 | 119-131 | 132-142 |
| | 15 | 1-66 | 67-135 | 136-168 | 169-213 | 214-234 | 235-330 | 331-360 | 361-390 |
| | 16 | 1-22 | 23-45 | 46-56 | 57-71 | 72-78 | 79-110 | 110-120 | 121-130 |

Specifically contemplated are engineered (non-naturally occurring) antibody molecules that bind specifically to and activate E-cadherin, which antibody molecules include a set of six CDRs identified herein as an E-cadherin binding set of CDRs. These include, for sentence, the sets of six CDRs derived from any one of monoclonal antibodies 19A11 (SEQ ID NOs: 17-22), 66E8 (SEQ ID NOs: 23-28), 56-4 (SEQ ID NOs: 29-34), or 18-5 (SEQ ID NOs: 35-50).

Also provided herein are functionally equivalent variants of the CDR sequences shown in SEQ ID NOs: 17-40, which also fall within the scope of the invention. As it is used herein, the term “functionally equivalent variant of a CDR sequence” refers to a sequence variant of a particular CDR sequence having substantially similar sequence identity with it and substantially maintaining its capacity to bind to its cognate antigen when part of an antibody or antibody fragment. For example, a functionally equivalent variant of a CDR sequence may be a polypeptide sequence derivative of said sequence including the addition, deletion or substitution of one or more amino acids.

Additional functionally equivalent variants of a CDR sequence include CDR sequences having at least 90%, at least 91%, at least 92%, at least 93%, at least 94%, at least 95%, at least 96%, at least 97%, at least 98% or at least 99% sequence identity with the corresponding amino acid sequences shown in one of SEQ ID NOs: 17-40. It is also contemplated that functionally equivalent variants of a CDR sequence include additions of at least 1 amino acid, or at least 2 amino acids, or at least 3 amino acids, or at least 4 amino acids, or at least 5 amino acids, or at least 6 amino acids at the N-terminus the C-terminus, or both at the N- and C-terminus of the corresponding amino acid sequence shown in one of SEQ ID NOs: 17-40. Likewise, it is also contemplated that variants include deletions of at least 1 amino acid, or at least 2 amino acids, or at least 3 amino acids, or at least 4 amino acids, or at least 5 amino acids, or at least 6 amino acids at the N-terminus, or at the C-terminus, or both at the N- and C-terminus of the corresponding amino acid sequence shown in one of SEQ ID NOs: 17-40.

Functionally equivalent variants of a CDR sequence will maintain at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 100%, at least 105%, at least 110%, at least 115%, at least 120%, at least 125%, at least 130%, at least 135%, at least 140%, at least 145%, at least 150%, at least 200% or more of the capacity of the corresponding amino acid sequence shown in one of SEQ ID NOs: 17-40 to bind to its cognate antigen (E-cadherin) when being part of an antibody or antibody fragment as provided herein. This capacity to bind to its cognate antigen may be determined as a value of affinity, avidity, specificity and/or selectivity of the antibody or antibody fragment to its cognate antigen.

E-cadherin is generally expressed in epithelial cells and tissues. Generally, epithelial tissues are sheets of cells that cover the surface of the body and the outside and inside of many internal organs. The main functions of epithelial tissues include protection, secretion, absorption, and filtration. Epithelial tissues are classified based on the cell shape of the apical cells and the number of layers of cells. Cell shape can be squamous, cuboidal, or columnar: A squamous cell has a flat, thin shape; a cuboidal cell has a box-like shape; and a columnar cell is tall and has a cuboid shape. A special case is made for transitional epithelial tissue found in the wall of the urinary bladder, where the cell shapes change based on how much the organ is stretched.

The number of cell layers in epithelial tissue determines whether the tissue is classified as simple (one cell layer thick) or stratified (containing two or more layers of cells). In epithelial tissue with many layers, such as in the skin and alimentary canal of the digestive system, the basal cells undergo cell division to replace exfoliated apical cells. Epithelial tissues are listed in Table 4.

TABLE 4

| Type of | | |
|---|---|---|
| Epithelial | | Representative |
| Tissue | Functions | Locations |
| Simple squamous | Material exchange via | Lung alveoli, kidney |
| | diffusion and filtration | glomeruli, blood and |
| | across membranes | lymphatic vessels, |
| | | membranes |
| Simple cuboidal | Secretion and absorption | Glands, ducts, kidney |
| | | tubules |
| Simple columnar | Secretion and absorption; | Alimentary tract of the |
| | ciliated cells propel | digestive system, |
| | secreted mucus across the | respiratory tract, uterus, |
| | apical surface, and | ducts, gallbladder |
| | absorptive cells contain | |
| | microvilli to increase | |
| | surface area. | |
| Pseudostratified | Secretion | Respiratory tract, ducts |
| columnar | | |
| Stratified | Protection of other tissues | Skin, lining of mouth, |
| squamous | from abrasions | esophagus, vagina |
| Stratified | Secretion of sweat, saliva, | Ducts of large glands |
| cuboidal | milk, and some hormones | |
| Stratified | Mucus secretion | Anus |
| columnar | | |
| Transitional | Stretching, protection from | Urinary bladder |
| | harsh conditions of urine | |

In particular embodiments, antibodies that specifically bind to and activate E-cadherin modulate cell adhesion or cell junctions in epithelial cells and not in endothelial cells. Endothelial cells can be distinguished from epithelial cells by structural and/or functional properties. Structural and functional properties of epithelial cells are discussed above. In particular embodiments, endothelial cells line the internal surface of the components of the circulatory system, such as the lumen of arteries, veins, blood capillaries, lymphatic vessels, and cavities of the heart. In particular embodiments, endothelial cells do not form more than one layer of cells. In particular embodiments, the main function of endothelial cells is to provide a slippery, non-sticky surface for the flow of fluids. In particular embodiments, endothelial cells include intermediate filaments instead of keratin filaments. In particular embodiments, the surface of an endothelial cell is smooth and lacks papillary projections.

An antibody that binds to and activates E-cadherin can include antibodies that enhance epithelial cell barrier function or inhibit loss of epithelial cell barrier permeability. In particular embodiments, an E-cadherin activating antibody can increase cell adhesion, reduce cell migration, and/or reduce cell invasion compared to a neutral antibody that binds E-cadherin but does not activate E-cadherin. The measurement of cell and barrier properties can be performed by numerous in vitro and in vivo experiments known to one of ordinary skill in the art and described herein.

In particular embodiments, the ability of an anti-E-cadherin antibody to enhance epithelial cell barrier function or inhibit loss of epithelial cell barrier permeability can be assessed in vitro by permeability assays and transepithelial electrical resistance (TEER) assays on in vitro barrier models. Permeability assays include tracer diffusion measurements in which the tracers are added in a donor compartment (i.e., the apical or basolateral side) and quantified in a received compartment (i.e., the opposite side) as a function of time. Tracers can include fluorescent dyes and radiolabeled markers. For TEER assays, agents that reduce barrier integrity can be added to in vitro cells, such as a monolayer cell culture of bronchial epithelial cells, and the ability of an anti-E-cadherin antibody to enhance or maintain barrier integrity against such agents can be tested. In particular embodiments, the TEER for a barrier system treated with an E-cadherin activating antibody is 10% higher, 15% higher, 20% higher, 25% higher, 30% higher, 35% higher, 40% higher, 45% higher, 50% higher, 55% higher, 60% higher, 65% higher, 70% higher, 75% higher, 80% higher, 85% higher, 90% higher, 95% higher, 96% higher, 97% higher, 98% higher, 99% higher, or more, as compared to the TEER for a barrier system treated with a neutral antibody that binds but does not activate E-cadherin.

In particular embodiments, the ability of an anti-E-cadherin antibody to enhance epithelial cell barrier function or inhibit loss of epithelial cell barrier permeability can be assessed in vivo using models of disrupted barrier function such as the adoptive T cell transfer mouse model of colitis or the IL-10 (IL-10^{-/-}) knockout mouse model of colitis. In mice treated with an anti-E-cadherin antibody, the ability to maintain the animal's weight or reduce weight loss, and/or reduce colon length shortening is an indication that the anti-E-cadherin antibody is an activating antibody. In particular embodiments, weight loss in a mouse treated with an E-cadherin activating antibody is reduced by 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, as compared to a mouse treated with a neutral antibody that binds but does not activate E-cadherin. In particular embodiments, colon length in a mouse treated with an E-cadherin activating antibody is 10% greater, 15% greater, 20% greater, 25% greater, 30% greater, 35% greater, 40% greater, 45% greater, 50% greater, 55% greater, 60% greater, 65% greater, 70% greater, 75% greater, 80% greater, 85% greater, 90% greater, 95% greater, 96% greater, 97% greater, 98% greater, 99% greater, or greater as compared to a mouse treated with a neutral antibody that binds but does not activate E-cadherin. Histopathological analysis of colon sections from mice in these models can also be performed and scores such as sum colitis scores, mean edema extent, mean mucosal thickness score, mean inflammation score, mean gland damage/loss score, mean erosion score, and/or mean neutrophil score can be obtained. In particular embodiments, a score from the histopathological analysis for a mouse treated with an E-cadherin activating antibody is reduced by 2×, 3×, 4×, 5×, 6×, 7×, 8×, 9×, 10×, 20×, 30×, 40×, 50×, or more, as compared to the score for a mouse treated with a neutral antibody that binds but does not activate E-cadherin.

In particular embodiments, the ability of an anti-E-cadherin antibody to increase cell adhesion can be assessed by in vitro cell culture. Cells can be seeded in wells of a plate, cultured, and treated with an anti-E-cadherin antibody. Cell adhesion activation can be determined by the extent of morphological change to compact epithelial appearance. In particular embodiments, cells treated with an E-cadherin activating antibody exhibit a more compact morphology by microscopy as compared to cells treated with a neutral antibody that binds but does not activate E-cadherin. A laminar flow cell adhesion assay can also be conducted, in which trypsinized cells are pretreated with an anti-E-cadherin antibody and allowed to attach to glass capillary tubes coated with E-cadherin. The cells are washed away at a particular flow rate and the percentage of cells remaining (adhered) after the wash is calculated. In particular embodiments, the percentage of cells adhered for cells treated with an E-cadherin activating antibody is 2% greater, 3% greater, 4% greater, 5% greater, 6% greater, 7% greater, 8% greater, 9% greater, 10% greater, 15% greater, 20% greater, 25% greater, 30% greater, 35% greater, 40% greater, 45% greater, 50% greater, 55% greater, 60% greater, 65% greater, 70% greater, 75% greater, 80% greater, 85% greater, 90% greater, 95% greater, 96% greater, 97% greater, 98% greater, 99% greater, or greater compared to the percentage of cells adhered for cells treated with a neutral antibody that binds but does not activate E-cadherin at a given time and a given flow rate.

In particular embodiments, the ability of an anti-E-cadherin antibody to decrease cell migration can be assessed by animal models of cancer metastases, such as the 4T1 mouse model of breast cancer, where metastasis of an E-cadherin—expressing mammary cell line from the mammary gland to the lung depends on reduced E-cadherin adhesive function. In particular embodiments, cell migration can be measured by the amount of metastatic tissue in a mouse modeling cancer metastasis. In particular embodiments, cell migration can be measured by the amount of circulating tumor cells (CTCs) in a mouse modeling cancer metastasis that has been injected with tumor cells expressing a reporter and E-cadherin for tracing of the tumor cells in the bloodstream. The number of CTCs can be calculated from mRNA levels of the reporter and/or E-cadherin expressed by the injected tumor cells. In particular embodiments, the amount of metastatic tissue, amount of CTCs, and/or mRNA levels of genes expressed by CTCs is/are decreased for a mouse treated with an E-cadherin activating antibody by 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, as compared to a mouse treated with a neutral antibody that binds but does not activate E-cadherin. In particular embodiments, the amount of metastatic tissue, amount of CTCs, and/or mRNA levels of genes expressed by CTCs is/are decreased for a mouse treated with an E-cadherin activating antibody by 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, 4.9×, 5×, or more, as compared to a mouse treated with a neutral antibody that binds but does not activate E-cadherin. In particular embodiments, cell migration can be assessed by an in vitro transwell assay, where cells are plated into upper wells of a transwell chamber containing a filter and allowed to migrate through the filter in test or control conditions for a period of time. Migration can be stopped by wiping the cells from the upper side of the chamber and fixing in methanol. Images of cell migration can be obtained by staining cells with crystal violet and visualizing the cells with an inverted microscope. In particular embodiments, cell migration is expressed as the number of cells that have migrated through the filter. In particular embodiments, the number of cells that have migrated after treatment with an E-cadherin activating antibody is 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, 96% less, 97% less, 98% less, 99% less, or less, as compared to the number of cells that have migrated for cells treated with a neutral antibody that binds but does not activate E-cadherin. In particular embodiments, the number of cells that have migrated after treatment with an E-cadherin activating antibody is 1.1× less, 1.2× less, 1.3× less, 1.4× less, 1.5× less, 1.6× less, 1.7× less, 1.8× less, 1.9× less, 2× less, 2.1× less, 2.2× less, 2.3× less, 2.4× less, 2.5× less, 2.6× less, 2.7× less, 2.8× less, 2.9× less, 3× less, 3.1× less, 3.2× less, 3.3× less, 3.4× less, 3.5× less, 3.6× less, 3.7× less, 3.8× less, 3.9× less, 4× less, 4.1× less, 4.2×less , 4.3× less, 4.4× less, 4.5× less, 4.6× less, 4.7× less, 4.8× less, 4.9× less, 5× less, 6× less, 7× less, 8× less, 9× less, 10× less, or less, as compared to the number of cells that have migrated for cells treated with a neutral antibody that binds but does not activate E-cadherin.

In particular embodiments, the ability of an anti-E-cadherin antibody to decrease cell invasion can be assessed in an in vitro setting. For example, 3D tumor organoids can be generated from primary tumors after mechanical disruption, enzymatic digestion, and embedding into a 3D gel. The 3D culturing can be in the presence of E-cadherin activating antibody and compared to control conditions of no antibody or of a neutral antibody that binds but does not activate E-cadherin. Cell invasion can be measured as a function of the longest invasive distance emanating from the cell spheroid body. In particular embodiments, the cell invasion distance for cells treated with an E-cadherin activating antibody is 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, 96% less, 97% less, 98% less, 99% less, or less, as compared to the cell invasion distance for cells treated with a neutral antibody that binds but does not activate E-cadherin. In particular embodiments, the cell invasion distance is 1.1× less, 1.2× less, 1.3× less, 1.4× less, 1.5× less, 1.6× less, 1.7× less, 1.8× less, 1.9× less, 2× less, 2.1× less, 2.2× less, 2.3× less, 2.4× less, 2.5× less, 2.6× less, 2.7× less, 2.8× less, 2.9× less, 3.0× less, 3.1× less, 3.2× less, 3.3× less, 3.4× less, 3.5× less, 3.6× less, 3.7× less, 3.8× less, 3.9× less, 4× less, 4.1× less, 4.2×less , 4.3× less, 4.4× less, 4.5× less, 4.6× less, 4.7× less, 4.8× less, 4.9× less, 5× less, 6× less, 7× less, 8× less, 9× less, 10× less, or less, for cells treated with an E-cadherin activating antibody compared to the cell invasion distance for cells treated with a neutral antibody that binds but does not activate E-cadherin. In particular embodiments, cell invasion can be assessed by an in vitro transwell assay similar to a transwell migration assay except that the filter is coated with diluted Matrigel to assess invasion into the Matrigel. In particular embodiments, the number of cells showing invasion after treatment with an E-cadherin activating antibody is 10% less, 15% less, 20% less, 25% less, 30% less, 35% less, 40% less, 45% less, 50% less, 55% less, 60% less, 65% less, 70% less, 75% less, 80% less, 85% less, 90% less, 95% less, 96% less, 97% less, 98% less, 99% less, or less, as compared to the number of cells showing invasion after treatment with a neutral antibody that binds but does not activate E-cadherin. In particular embodiments, the number of cells showing invasion after treatment with an E-cadherin activating antibody is 1.1× less, 1.2× less, 1.3× less, 1.4× less, 1.5× less, 1.6× less, 1.7× less, 1.8× less, 1.9× less, 2× less, 2.1× less, 2.2× less, 2.3× less, 2.4× less, 2.5× less, 2.6× less, 2.7× less, 2.8× less, 2.9× less, 3× less, 3.1× less, 3.2× less, 3.3× less, 3.4× less, 3.5× less, 3.6× less, 3.7× less, 3.8× less, 3.9× less, 4× less, 4.1× less, 4.2×less , 4.3× less, 4.4× less, 4.5× less, 4.6× less, 4.7× less, 4.8× less, 4.9× less, 5× less, 6× less, 7× less, 8× less, 9× less, 10× less, or less, as compared to the number of cells showing invasion after treatment with a neutral antibody that binds but does not activate E-cadherin.

In particular embodiments, the ability of an anti-E-cadherin antibody to activate E-cadherin can be assessed by assays that measure the extent of apoptosis in cells. For example, a terminal deoxynucleotidyl transferase dUTP nick end labeling (TUNEL) assay can detect DNA fragmentation in apoptotic cells by labeling the 3′ hydroxyl termini in double-stranded DNA breaks. As another example, cleaved caspase-3, a specific apoptosis marker, can be measured in cells by immunofluorescence staining. As a further example, mRNA levels of pro-apoptotic markers (e.g., Bax) and anti-apoptotic markers (e.g., Bcl-xL) can be measured in cells by quantitative RT-PCR. In particular embodiments, the percentage of TUNEL+ cells in a population of cells treated with an E-cadherin activating antibody is increased 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, 4.9×, 5×, or more, as compared to the percentage of TUNEL+ cells in a population of cells treated with a neutral antibody that binds but does not activate E-cadherin. In particular embodiments, the percentage of cleaved caspase-3 positive cells in a population of cells treated with an E-cadherin activating antibody is increased 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, 4.9×, 5×, or more, as compared to the percentage of cleaved caspase-3 positive cells in a population of cells treated with a neutral antibody that binds but does not activate E-cadherin. In particular embodiments, the mRNA level of a pro-apoptotic marker in cells treated with an E-cadherin activating antibody is increased 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, 4.9×, 5×, or more, as compared to the mRNA level of a pro-apoptotic marker in cells treated with a neutral antibody that binds but does not activate E-cadherin. In particular embodiments, the mRNA level of an anti-apoptotic marker in cells treated with an E-cadherin activating antibody is decreased 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, 4.9×, 5×, or more, as compared to the mRNA level of an anti-apoptotic marker in cells treated with a neutral antibody that binds but does not activate E-cadherin. In particular embodiments, cell proliferation is not affected by treatment of cells with an E-cadherin activating antibody as measured by assessment of proliferation markers such as Ki67 and pHistone3.

The terms “polypeptide,” “peptide” and “protein” are used interchangeably herein to refer to a polymer of amino acid residues. These terms apply equally to amino acid polymers in which one or more amino acid residue is an artificial chemical mimetic of a corresponding naturally occurring amino acid, as well as to naturally occurring amino acid polymers and non-naturally occurring amino acid polymer.

The term amino acid encompasses naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The phrase conservatively modified variant(s) applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein or protein domain. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one type of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

(II) Production of Antibodies and Antibodies Variations

An antibody is a type of binding agent, which is a molecule that can bind a target ligand, for instance on the surface of a cell or in a biological sample. The term antibody includes both whole antibodies and functional (that is, maintaining significant and specific target binding) fragments thereof. The terms “antibody” and “immunoglobulin” are used interchangeably herein and are well understood by those in the field. Those terms refer to a protein including one or more polypeptides that specifically binds an antigen.

One form of antibody includes the basic structural unit of an antibody. This form is a tetramer and includes two pairs of antibody chains, each pair having one light and one heavy chain. In each pair, the light and heavy chain variable regions are together responsible for binding to the antigen recognized by that antibody, and the constant regions are responsible for the antibody effector functions.

The recognized immunoglobulin polypeptides include the kappa and lambda light chains and the alpha, gamma (IgG1, IgG2, IgG3, IgG4), delta, epsilon and mu heavy chains or equivalents in other species. Full-length immunoglobulin “light chains” (of 25 kDa or 214 amino acids) include a variable region of 110 amino acids at the NH₂-terminus and a kappa or lambda constant region at the COOH-terminus. Full-length immunoglobulin “heavy chains” (of 50 kDa or 446 amino acids), similarly include a variable region (of 116 amino acids) and one of the aforementioned heavy chain constant regions, e.g., gamma (of 330 amino acids).

Particular embodiments of antibodies and immunoglobulins include antibodies or immunoglobulins of any isotype, fragments of antibodies which retain specific binding to antigen, including, Fab, Fv, scFv, and Fd fragments, chimeric antibodies, humanized antibodies, single-chain antibodies, and fusion proteins including an antigen-binding portion of an antibody and a non-antibody protein. The antibodies may be detectably labeled, e.g., with a radioisotope, an enzyme which generates a detectable product, a fluorescent protein, a fluorescent molecule, or a stable elemental isotope and the like. The antibodies may be further conjugated to other moieties, such as members of specific binding pairs, e.g., biotin (member of a biotin-avidin specific binding pair), and the like. Also encompassed by the term are Fab′, Fv, F(ab′)₂, and other antibody fragments that retain specific binding to their cognate antigen, and monoclonal antibodies. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554, 1990).

Antibodies may exist in a variety of other forms including, for example, bi-functional (i.e. bi-specific) hybrid antibodies (e.g., Lanzavecchia et al., Eur. J. Immunol. 17: 105, 1987) and in single chains (e.g., Huston et al., Proc. Natl. Acad. Sci. U.S.A. 85: 5879-5883, 1988; and Bird et al., Science 242: 423-426, 1988). See, generally, Hood et al. (1984) “Immunology”, N.Y., 2nd ed., and Hunkapiller & Hood (Nature 323: 15-16, 1986).

An immunoglobulin light or heavy chain variable region includes of a “framework” region (FR) interrupted by three hypervariable regions, also called “complementarity determining regions” or “CDRs”. The extent of the framework region and CDRs has been precisely defined (see, “Sequences of Proteins of Immunological Interest” E. Kabat et al. (1991) US Department of Health and Human Services). In particular embodiments, the numbering of an antibody amino acid sequence can conform to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. Table 3 provides specific information on the framework and CDR positions of certain E-cadherin activating antibodies described herein; additional information is provided in Table 1.

The phrase “humanized antibody” refers to an antibody derived from a non-human antibody, such as a murine antibody, that retains the antigen-binding properties of the parent antibody, but which is less immunogenic in humans. This may be achieved by various methods, including (a) grafting the entire non-human variable domains onto human constant regions to generate chimeric antibodies; (b) grafting only the non-human complementarity determining regions (CDRs) into human framework and constant regions with or without retention of critical framework residues; and (c) transplanting the entire non-human variable domains, but “cloaking” them with a human-like section by replacement of surface residues. Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. It is further important that antibodies are humanized with retention of high affinity for the antigen and other favorable biological properties. To achieve this goal, humanized antibodies are prepared by a process of analysis of the parental sequences and various conceptual humanized products using three-dimensional models of the parental and humanized sequences. A further step in this approach, to make an antibody more similar to humans, is to prepare the so called primatized antibodies, i.e. a recombinant antibody which has been engineered to contain the variable heavy and light domains of a monkey (or other primate) antibody, in particular, a cynomolgus monkey antibody, and which contains human constant domain sequences, preferably the human immunoglobulin gamma 1 or gamma 4 constant domain (or PE variant). Methods for humanizing (or more generally primatizing) non-human antibodies are well known in the art. Humanization can be essentially performed following the method of Winter and co-workers (see, e.g., Jones et al., Nature 321:522-525, 1986; Riechmann et al., Nature 332:323-327, 1988; Verhoeyen et al., Science 239:1534-1536, 1988; and Presta, Curr. Op. Struct. Biol. 2:593-596, 1992), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Example chimeric antibodies are antibodies whose light and heavy chain genes have been constructed, typically by genetic engineering, from antibody variable and constant region genes belonging to different species. For example, the variable segments of the genes from a rabbit monoclonal antibody may be joined to human constant segments, such as gamma 1 and gamma 3. Preferred antibodies of this disclosure, and for use according methods provided herein, include humanized and/or chimeric monoclonal antibodies.

In one embodiment, the antibody is conjugated to an “effector” moiety. The effector moiety can be any number of molecules, including labeling moieties (such as radioactive labels or fluorescent labels), or a therapeutic moiety. Such effector moieties include a cytokine, a second antibody or an enzyme. The effector moiety may also serve as a targeting moiety, providing delivery of the antibody molecule to which it is conjugated to a specific tissue, cell type, or cell. By way of example, targeting moieties that direct delivery of a conjugated activating E-cadherin-specific antibody molecule to an epithelial tissue, such as an airway epithelium or an intestinal epithelium, are contemplated. Such epithelium-targeting moieties may include antibody or other ligand-binding domains that are preferential for or specific for one or more markers of epithelial cells, or more particularly of the specific type of tissue to be targeted.

The phrase “specifically (or selectively) binds” or “specifically (or selectively) immunoreactive with,” when referring to a protein or peptide, refers to a binding reaction of an antibody or functional fragment thereof that is determinative of the presence of the protein, often in a heterogeneous population of proteins and other biologics. Thus, under designated immunoassay conditions, the specified antibodies bind to a particular protein (such as E-cadherin) at least two times the background and more typically more than 10 to 100 times background. Specific binding to an antibody under such conditions requires an antibody that is selected for its specificity for a particular protein. For example, variant antibody molecules can be selected to obtain only those antibody molecules that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

(III) Pharmaceutical Compositions and Administration Formulations

Provided herein are compositions for use in activating E-cadherin, for instance in order to treat a disease or condition characterized by disruption of normal cell adhesion and/or cell junctions. Such diseases and conditions include cancer metastasis, inflammatory bowel disease, and inflammation of other epithelial organs. Appropriate E-cadherin activating antibodies (including mouse anti-human E-cadherin monoclonal antibody mAB-1_19A11, mouse anti-human E-cadherin monoclonal antibody 66E8; rabbit anti-mouse E-cadherin monoclonal antibody mAb-1_56-4; and rabbit anti-mouse E-cadherin monoclonal antibody mAb-2_18-5) and functional fragments and derivatives thereof are described herein. In various method embodiments, the antibodies of the present invention are preferably intact antibody molecules including the Fc region. Such intact antibodies will have longer half-lives than smaller fragment antibodies (e.g. Fab) and are generally more suitable for intravenous, intraperitoneal, intramuscular, intracavity, subcutaneous, or transdermal administration. It is also believed that at least some of the function provided by activating E-cadherin antibodies is a result of steric interaction that may, at least in part, be influenced by the form of the antibody molecule being employed.

When formulated in a pharmaceutical composition, a therapeutic compound (such as an antibody or functional fragment thereof) can be admixed with a pharmaceutically acceptable carrier or excipient. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human or veterinary subject.

The term “pharmaceutically acceptable derivative” as used herein means any pharmaceutically acceptable salt, solvate or prodrug, e.g. ester, of the desired active agent, which upon administration to the recipient is capable of providing (directly or indirectly) the desired active agent, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol. 1: Principles and Practice. Pharmaceutically acceptable derivatives include salts, solvates, esters, carbamates, and phosphate esters.

The terms “pharmaceutically acceptable salts” and “pharmaceutically acceptable carrier” include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., J Pharma Sci 66:1-19, 1977). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention. For a discussion of how salt type and concentration may influence monoclonal antibodies, see for instance Arosio et al. (Biophys Chem 168-169:19-27, 2012).

While it is possible to use a composition for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, pharmaceutical composition or formulation includes at least one active composition, or a pharmaceutically acceptable derivative thereof, in association with a pharmaceutically acceptable excipient, diluent and/or carrier. The excipient, diluent and/or carrier is “acceptable” in the sense of being compatible with the other ingredient(s) of the formulation and not significantly deleterious to the recipient thereof.

Any composition formulation disclosed herein can advantageously include any other pharmaceutically acceptable carriers which include those that do not produce significantly adverse, allergic, or other untoward reactions that outweigh the benefit of administration, whether for research, prophylactic and/or therapeutic treatments. Exemplary pharmaceutically acceptable excipients, diluents, and carriers for therapeutic use are well known in the pharmaceutical art, and are described, for example, in Remington: The Science and Practice of Pharmacy. Lippincott Williams & Wilkins (A.R., Gennaro edit. 2005), and in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies. The pharmaceutical excipient(s), diluent(s), and carrier(s) can be selected with regard to the intended route of administration and standard pharmaceutical practice.

Such pharmaceutical formulations may be presented for use in a conventional manner with the aid of one or more suitable excipients, diluents, and carriers. Pharmaceutically acceptable excipients assist or make possible the formation of a dosage form for a bioactive material and include diluents, binding agents, lubricants, glidants, disintegrants, coloring agents, and other ingredients. Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used. An excipient is pharmaceutically acceptable if, in addition to performing its desired function, it is non-toxic, well tolerated upon ingestion, and does not interfere with absorption of bioactive materials.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol, or mannitol.

Exemplary stabilizers include organic sugars, polyhydric sugar alcohols, polyethylene glycol; sulfur-containing reducing agents, amino acids, low molecular weight polypeptides, proteins, immunoglobulins, hydrophilic polymers, or polysaccharides.

The pharmaceutical compositions can be formulated for administration in any convenient way for use in human or veterinary medicine. Exemplarily modes of administration are discussed herein.

A “therapeutically effective amount” or “therapeutically effective dose” means the amount of a compound that, when administered to a subject for treating a state, disorder or condition, is sufficient to affect such state, disorder, or condition. The “therapeutically effective amount” will vary depending on the compound, the disease and its severity and the age, weight, physical condition and responsiveness of the mammal to be treated. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). In certain cases, “therapeutically effective amount” is used to mean an amount or dose sufficient to modulate, e.g., increase or decrease a desired activity e.g., by 10%, by 50%, or by 90%. Generally, a therapeutically effective amount is sufficient to cause an improvement in a clinically significant condition in the host following a therapeutic regimen involving one or more therapeutic agents. The concentration or amount of the active ingredient depends on the desired dosage and administration regimen, as discussed herein. Suitable dosages may range from 0.01 mg/kg to 100 mg/kg of body weight per day, week, or month.

The actual dose amount administered to a particular subject can be determined by a physician, veterinarian, or researcher taking into account parameters such as physical, physiological and psychological factors including target, body weight, stage of cancer, the type of cancer, previous or concurrent therapeutic interventions, idiopathy of the subject, and route of administration.

Amounts effective for this use will depend on the severity of the disease and its location, particularly when a metastatic site is implicated, and the weight and general state of the patient being treated. Generally dosages range from 0.01 mg/kg to 100 mg/kg host body weight of monoclonal antibody per day, with dosages of from 0.1 mg/kg to 10 mg/kg per day being more commonly used, and for instance dosages of 3-7 mg/kg. Maintenance dosages over a prolonged period of time may be adjusted as necessary. The dosages, however, may be varied depending upon the requirements of the patient, the severity of the condition being treated, and the compound being employed. For example, dosages can be empirically determined considering the type and stage of cancer diagnosed in a particular patient. The dose administered to a patient, in the context of the present disclosure should be sufficient to result in or provide a beneficial therapeutic response in the patient over time. The size of the dose also will be determined by the existence, nature, and extent of any adverse side-effects that accompany the administration of a particular vector, or transduced cell type in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

Exemplary doses can include 0.05 mg/kg to 10.0 mg/kg of the active compounds (antibodies or antibody fragments) disclosed herein. The total daily dose can be 0.05 mg/kg to 30.0 mg/kg of an agent administered to a subject one to three times a day, including administration of total daily doses of 0.05-3.0, 0.1-3.0, 0.5-3.0, 1.0-3.0, 1.5-3.0, 2.0-3.0, 2.5-3.0, and 0.5-3.0 mg/kg/day of administration forms of a drug using 60-minute oral, intravenous or other dosing. In one particular example, doses can be administered QD or BID to a subject with, e.g., total daily doses of 1.5 mg/kg, 3.0 mg/kg, 4.0 mg/kg, 5.0 mg/kg, or 7.5 mg/kg of a composition with up to 92-98% wt/v of the compounds disclosed herein.

Additional useful doses can often range from 0.1 to 5 μg/kg or from 0.5 to 1 μg/kg. In other examples, a dose can include 1 μg/kg, 10 μg/kg, 20 μg/kg, 40 μg/kg, 80 μg/kg, 200 μg/kg, 0.1 to 5 mg/kg or from 0.5 to 1 mg/kg. In other examples, a dose can include 1 mg/kg, 10 mg/kg, 20 mg/kg, 40 mg/kg, 80 mg/kg, 200 mg/kg, 400 mg/kg, 450 mg/kg, or more.

Therapeutic materials of the present disclosure may be employed in serious disease states, that is, life-threatening or potentially life-threatening situations. In such cases, in view of the minimization of extraneous substances and general lack of immunogenicity when a monoclonal antibody of the invention is employed to treat human hosts, it is possible and may be felt desirable by the treating physician to administer substantial excesses of these compositions.

As will be appreciated by those of skill in the art, specific dosages will be influenced by the pharmacokinetics of the active compound.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly).

In another embodiment, the active ingredient(s) can be delivered in a vesicle, in particular a liposome (see Langer, Science, 1990;249:1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327). Vesicle-mediated delivery and any other modes (including newly developed modes) of drug delivery can be applied to the herein-described pre-conditioning regimen. As will be recognized by those of ordinary skill, the appropriate dose is re-standardized for each mode.

The effective amounts of compounds containing active agents include doses that partially or completely achieve the desired therapeutic, prophylactic, and/or biological effect. The actual amount effective for a particular application depends on the condition being treated and the route of administration. The effective amount for use in humans can be determined from animal models. For example, a dose for humans can be formulated to achieve circulating and/or gastrointestinal concentrations that have been found to be effective in animals.

For administration, therapeutically effective amounts (also referred to herein as doses) can be initially estimated based on results from in vitro assays and/or animal model studies. Such information can be used to more accurately determine useful doses in subjects of interest. Useful pre-clinical tests include pharmacodynamic analyses, toxicity analyses, and so forth.

The pharmaceutical compositions may also include other biologically active compounds, in addition to one or more of the herein provided antibodies or active fragments thereof.

Compositions can be administered with one or more anesthetics including ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and/or phenazopyridine.

In particular embodiments that include treating or preventing a cancer (including for instance a cancer metastasis), the compositions disclosed herein can be used in conjunction with other cancer treatments, such as chemotherapeutic agents, radiation therapy, and/or immunotherapy. The compositions described herein can be administered simultaneously with or sequentially with another treatment within a selected time window, such as within 10 minutes, 1 hour, 3 hour, 10 hour, 15 hour, 24 hour, or 48 hour time windows or when the complementary treatment is within a clinically-relevant therapeutic window.

In particular embodiments that include treating or ameliorating an inflammatory condition or disease, the compositions disclosed herein can be used in conjunction with one or more other anti-inflammatory agents. Anti-inflammatory agent include naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, Rho-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone or betamethasone and other glucocorticoids.

Also contemplated for use in combination therapies are anti-inflammatory cytokines. Cytokines are small secreted proteins or factors (5 to 20 kD) that have specific effects on cell-to-cell interactions, intercellular communication, or the behavior of other cells. Cytokines are produced by lymphocytes, especially T_{H}1 and T_{H}2 lymphocytes, monocytes, intestinal macrophages, granulocytes, epithelial cells, and fibroblasts (reviewed in Rogler & Andus, World J. Surg. 22(4):382-389, 1998; Galley & Webster, Br. J. Anaesth. 77:11-16, 1996). Some cytokines are pro-inflammatory (e.g., tumor necrosis factor [TNF]-α, interleukin [IL]-1(α and β), IL-6, IL-8, IL-12, or leukemia inhibitory factor [LIF]); others are anti-inflammatory (e.g., IL-1 receptor antagonist [IL-Ira], IL-4, IL-10, IL-11, and transforming growth factor [TGF]-β). However, there may be overlap and functional redundancy in their effects under certain inflammatory conditions. Optionally, treating further includes administering to the human subject an anti-inflammatory cytokine to accelerate or further improve the symptom(s) of inflammatory bowel syndrome, or another inflammatory condition involving defects in the permeability of the epithelial boundary. Useful anti-inflammatory cytokines include human IL-4, L-10, IL-11, or TGF-β, derived from a human source or a transgenic non-human source expressing a human gene. The anti-inflammatory cytokine is preferably injected or infused intravenously or subcutaneously, and may be administered concurrently with, before, or after an activating anti-E-cadherin antibody.

Principal anti-inflammatory cytokines and cytokine inhibitors are listed in Tables 5 and 6. The functional definition of an anti-inflammatory cytokine is the ability of the cytokine to inhibit the synthesis of IL-1, tumor necrosis factor (TNF), and other major proinflammatory cytokines.

TABLE 5
Cytokines with anti-inflammatory activities

| Cytokines | Major Activities |
|---|---|
| II-1ra | Specific inhibitor of IL-1α and IL-1β mediated cellular |
| | activation at the IL-1 cellular receptor level |
| IL-4 | Promotes T_{H}2 lymphocyte development; inhibition of |
| | LPS-induced proinflammatory cytokines synthesis |
| IL-6 | Inhibition of TNF and IL-1 production by macrophages |
| IL-10 | Inhibition of monocyte/macrophage and neutrophil |
| | cytokine production and inhibition of T_{H}1-type |
| | lymphocyte responses |
| IL-11 | Inhibits proinflammatory cytokines response by |
| | monocyte/macrophages and promotes T_{H}2 |
| | lymphocyte response |
| IL-13 | Shares homology with IL-4 and shares IL-4 receptor; |
| | attenuation of monocyte/macrophage function |
| TGF-β | Inhibition of monocyte/macrophage MHC, class II |
| | expression and proinflammatory cytokines synthesis |

TABLE 6
Soluble Cytokine Receptors with Anti-inflammatory Activities

| Soluble receptor | Major Activities |
|---|---|
| Soluble TNF receptor | Binds to TNF trimers in the circulation, |
| p55 (sTNFRI or | preventing membrane-bound TNF |
| sTNFRp55) | receptor-TNF ligand interactions |
| Soluble TNF receptor | Binds to TNF trimers in the circulation, |
| p75 (sTNFRII or | preventing membrane-bound TNF |
| sTNFRP75) | receptor-TNF ligand interactions |
| Soluble IL-1 receptor | Binds to circulating IL-1 ligands in the |
| type 2 (sIL-1RII) | plasma, preventing IL-1β from binding |
| | to the IL-1 receptor type 1 |
| Membrane-bound | Decoy receptor that lacks intracellular |
| IL-1 receptor type 2 | signaling function and competes with |
| (mIL-1RII) | type 1 IL-1R for IL-1 ligand binding at |
| | the cell membrane |
| IL-18 binding protein | Soluble extracellular domain of IL-18 |
| (IL-18BP) | receptor that function as a decoy |
| | receptor and binds circulating IL-18 |

IL-1ra is a 152-amino-acid protein that functions as a specific inhibitor of the two other functional members of the IL-1 family, IL-1a and IL-1b. IL-1ra blocks the action of IL-1a and IL-1b functional ligands by competitive inhibition at the IL-1 receptor level. IL-1ra binds with equal or greater affinity than does IL-1a and IL-1b to the type 1 (80 kd) membrane-bound IL-1 receptor. IL-1ra does not bind with high affinity to the type II (68 kd) IL-1 receptor.

IL-1ra is produced by monocytes and macrophages and is released into the systemic circulation in >100-fold excess than either IL-1a or IL-1b after lipopolysaccharide (LPS) stimulation in human volunteers. The synthesis of IL-1ra and IL-1b are differentially regulated at their own promoter sites. Although bacterial LPS stimulates the synthesis of both IL-1b and IL-1ra, other stimuli cause differential release of IL-1ra and IL-1b. The anti-inflammatory cytokines IL-4, IL-6, IL-10, and IL-13 inhibit the synthesis of IL-1b, yet they stimulate the synthesis of IL-1ra.

IL-4 is a highly pleiotropic cytokine that is able to influence Th cell differentiation. Early secretion of IL-4 leads to polarization of Th cell differentiation toward T_{H}2-like cells. T_{H}2-type cells secrete their own IL-4, and subsequent autocrine production of IL-4 supports cell proliferation. IL-4 is able to affect a variety of structural cells. It can potentiate proliferation of vascular endothelium and skin fibroblasts yet decrease proliferation of adult human astrocytes and vascular smooth muscle cells. In addition, IL-4 induces a potent cytotoxic response against tumors. In a study of 63 patients with stage IV non-small cell lung cancer, data on treatment with recombinant human IL-4 seemed to suggest a possible dose-related response. IL-4 may act by stabilizing disease and modifying tumor growth rates in addition to inducing tumor shrinkage and cell death without causing severe side effects, suggesting a possible adjuvant role for IL-4 in the treatment of malignant diseases.

IL-6 has long been regarded as a proinflammatory cytokine induced by LPS along with TNF-a and IL-1. IL-6 is often used as a marker for systemic activation of proinflammatory cytokines. Like many other cytokines, IL-6 has both proinflammatory and anti-inflammatory properties. Although IL-6 is a potent inducer of the acute-phase protein response, it has anti-inflammatory properties as well. IL-6, like other members of the gp130 receptor ligand family, acts predominantly as an anti-inflammatory cytokine. IL-6 down-regulates the synthesis of IL-1 and TNF.

IL-6 attenuates the synthesis of the proinflammatory cytokines while having little effect on the synthesis of anti-inflammatory cytokines such as IL-10 and transforming growth factor-b (TGF-β). IL-6 induces the synthesis of glucocorticoids and promotes the synthesis of IL-1ra and soluble TNF receptor release in human volunteers. At the same time, IL-6 inhibits the production of proinflammatory cytokines such as GM-CSF, IFN-γ, and MIP-2. The net result of these immunologic effects place IL-6 the anti-inflammatory cytokine group.

IL-10 is the most important anti-inflammatory cytokine found within the human immune response. It is a potent inhibitor of T_{H}1 cytokines, including both IL-2 and IFN-y. This activity accounts for its initial designation as cytokine synthesis inhibition factor. In addition to its activity as a T_{H}2 lymphocyte cytokine, IL-10 is also a potent deactivator of monocyte/macrophage proinflammatory cytokine synthesis. After engaging its high-affinity 110-kd cellular receptor, IL-10 inhibits monocyte/macrophage-derived TNF-a, IL-1, IL-6, IL-8, IL-12, granulocyte colony-stimulating factor, MIP-1 a, and MIP-2a.

IL-11 has been shown to attenuate IL-1 and TNF synthesis from macrophages by up-regulating inhibitory NF-kB (inhibitory NF-kB) synthesis in monocyte/macrophage cell lines. Inhibitory NF-kB prevents NF-kB from translocating to the nucleus where NF-kB functions as a transcriptional activator for the proinflammatory cytokines. IL-11 has also been shown to inhibit the synthesis of IFN-g and IL-2 by CD41 T cells. IL-11 functions as a Th2-type cytokine, with induction of IL-4 and Inhibition of Th1-type cytokines. IL-11 does not induce the synthesis of IL-10 or TGF-b. This indicates that IL-11 is a direct inhibitor of Th1 lymphocytes and does not act indirectly through induction of IL-10.

IL-13 and IL-4 share a common cellular receptor (IL-4 type 1 receptor), and this accounts for many of the similarities between these two anti-inflammatory cytokines. IL-4 and IL-13 share only 20% to 25% primary amino acid homology, but the majora-helical regions that are essential for their activity are highly homologous. IL-13 can down-regulate the production of TNF, IL-1, IL-8, and MIP-1a by monocytes and has profound effects on expression of surface molecules on both monocytes and macrophages.

Like many cytokines, TGF-β has both pro- and anti-inflammatory effects. It functions as a biological switch, antagonizing or modifying the action of other cytokines or growth factors. The presence of other cytokines may modulate the cellular response to TGF-β, and the effect may differ depending on the activation state of the cell. TGF-β is capable of converting an active site of inflammation into one dominated by resolution and repair. TGF-β often exhibits disparate effects with immune-enhancing activity in local tissues and immune-suppressive activity in the systemic circulation. TGF-β1 suppresses the proliferation and differentiation of T cells and B cells and limits IL-2, IFN-g, and TNF production. TGF-β1 acts as a monocyte/macrophage deactivator in a manner similar to IL-10. However, TGF-β is less potent an inhibitor than IL-10 and has little or no effect on IL-1 production. The severe and uncontrolled inflammatory reactions observed in the TGF-β knockout mouse attests to the physiologic role of TGF-b as an endogenous anti-inflammatory cytokine.

There are also many soluble cytokine receptors as anti-inflammatory molecules. Such as: type 1 (p55) and type 2 (p75) receptors for human TNF-α.

The anti-E-cadherin antibodies, immunoconjugates, and related compositions described herein can be administered (on their own or as part of a combination therapy) by a variety of routes. A therapeutically effective amount of the desired active agent(s) can be formulated in a pharmaceutical composition to be introduced parenterally, transmucosally (e.g., orally, nasally, or rectally), or transdermally. In some embodiments, administration is parenteral, for instance, via intravenous injection, or intra-arteriole, intramuscular, intradermal, subcutaneous, intraperitoneal, intraventricular, and intracranial administration. The administered may be as a bolus or by continuous infusion over a period of time, by intramuscular, intraperitoneal, intracerobrospinal, subcutaneous, intra-articular, intrasynovial, intrathecal, oral, topical, or inhalation routes. In certain embodiments, for instance those involved in treatment of inflammatory conditions that impact joints, the pharmaceutical composition may be administered directly to the synovium, synovial fluid or joint capsule by injection preferably with a syringe. Administration may be local or systemic; the choice may be influenced by the condition being treated, as well as the active agent(s) and compositions being administered.

For injection, compositions can be made as aqueous solutions, such as in buffers such as Hanks' solution, Ringer's solution, or physiological saline. The solutions can contain formulatory agents such as suspending, stabilizing and/or dispersing agents. Alternatively, the composition can be in lyophilized and/or powder form for constitution with a suitable vehicle, e.g., sterile pyrogen-free water, before use.

Compositions can also be formulated for oral administration. For ingestion, compositions can take the form of powders, tablets, pills, lozenges, sprays, liquids, and capsules formulated in conventional manners. Ingestible compositions can be prepared using conventional methods and materials known in the pharmaceutical art. For example, U.S. Pat. Nos. 5,215,754 and 4,374,082 relate to methods for preparing swallowable compositions. U.S. Pat. No. 6,495,177 relates to methods to prepare chewable supplements with improved mouthfeel. U.S. Pat. No. 5,965,162, relates to compositions and methods for preparing comestible units which disintegrate quickly in the mouth. It is recognized that antibodies when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

Ingestible compositions may have a shape containing no sharp edges and a smooth, uniform and substantially bubble free outer coating. Coatings of ingestible compositions can be derived from a polymeric film. Such film coatings reduce the adhesion of the compositions to the inner surface of the mouth and can aid in masking potential unpleasant tastes. Coatings can also protect the compositions from atmospheric degradation. Exemplary polymeric films include vinyl polymers, cellulosics, acrylates and methacrylates, natural gums and resins such as zein, gelatin, shellac and acacia. Other common excipients used in ingestible compositions include sucrose, fructose, lactose, glucose, lycasin, xylitol, lactitol, erythritol, mannitol, isomaltose, dextrose, polydextrose, dextrin, compressible cellulose, compressible honey, compressible molasses, fondant or gums, vegetable oils, animal oils, alkyl polysiloxanes, corn starch, potato starch, pre-gelatinized starches, stearic acid, calcium stearate, magnesium stearate, zinc stearate, benzoic acid, and colorants.

For administration by inhalation (e.g., nasal or pulmonary), the compositions can be formulated as aerosol sprays for pressurized packs or a nebulizer, with the use of suitable propellants, e.g. dichlorodifluoromethane, trichlorofluoromethane, or dichlorotetra-fluoroethane.

(IV) Methods of Use:

With the provision herein of therapeutic, activating E-cadherin antibodies and biologically functional fragments thereof, there are now enabled methods of treating, ameliorating, and/or preventing various diseases and conditions that are influenced by, or that influence, cell adhesion. In particular embodiments, antibodies for therapeutic use include mAb 19A11, 66E8, as well as humanized versions and functional fragments thereof, that specifically bind to and activate human E-cadherin. Particularly contemplated are treatments of conditions in which it would be beneficial to increase cell adhesion (or decrease loss of cell adhesion), including cancer metastasis as well as various inflammatory disease and conditions. Specifically contemplated are diseases and conditions that involve respiratory tissue inflammation, such as ARDS (no matter the cause), as well as bacterial and viral infections of the lungs that impact or are influenced by cell adhesion and/or the health of the epithelial barrier cells, tissue, and function.

Testing and Detection:

In certain embodiments, an activating E-cadherin antibody molecule provided herein (or another E-cadherin-specific antibody molecule) is used to determine the presence (or absence) and/or amount of E-cadherin in a biological sample. “E-Cadherin” refers to nucleic acids, e.g., gene, pre-mRNA, mRNA, and polypeptides, polymorphic variants, alleles, mutants, and interspecies homologs that: (1) have an amino acid sequence that has greater than 80%, 85%, 90%, 91%, 92%, 93%, 94%, 95%,96%, 97%, 98% or 99% or greater amino acid sequence identity, preferably over a region of over a region of at least about 25, 50, 100, 200, 500, 1000, or more amino acids, to a polypeptide encoded by a respectively referenced nucleic acid or an amino acid sequence recognized to be E-cadherin, for example, as depicted in GenBank Accession Nos. NM 004360 (E-Cadherin mRNA), and NP_004351 (E-Cadherin protein); (2) specifically bind to an antibody known to recognize E-cadherin; immunogenic fragments respectively thereof, and conservatively modified variants respectively thereof; (3) specifically hybridize under stringent hybridization conditions to a nucleic acid encoding a referenced amino acid sequence as depicted in GenBank Accession No. NP_004351 (E-Cadherin protein), and conservatively modified variants respectively thereof; (4) have a nucleic acid sequence that has greater than 95%, 96%, 97%, 98%, 99%, or higher nucleotide sequence identity, preferably over a region of at least about 25, 50, 100, 150, 200, 250, 500, 1000, or more nucleotides, to a reference nucleic acid sequence as shown in GenBank Accession No. NM_004360 (E-Cadherin mRNA).

The phrase “biological sample” includes sections of tissues such as biopsy and autopsy samples, and frozen sections taken for histological purposes. Biological samples also include blood and blood fractions or products (e.g., serum, plasma, platelets, red blood cells, and the like), sputum, tissue, cultured cells, e.g., primary cultures, explants, and transformed cells, stool, urine, etc. A biological sample is typically obtained from a eukaryotic organism, for instance a mammal such as a primate (e.g., monkey, chimpanzee, or human); cow or other livestock animal; dog; cat; rodent (e.g., guinea pig, rat, mouse, rabbit); bird; reptile; or fish.

The term “biopsy” refers to the process of removing a tissue sample for diagnostic or prognostic evaluation, as well as referring to the tissue sample itself. Any biopsy technique known in the art can be applied to the methods disclosed herein. The biopsy technique applied will depend on the tissue type to be evaluated (i.e., prostate, lymph node, liver, bone marrow, blood cell), as well as the size and type of the tumor (i.e., solid or suspended (i.e., blood or ascites)), among other factors. Representative biopsy techniques include excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. Certain embodiments may employ a “core-needle biopsy” of a tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70.

Detection and measurement (assay) can be carried out by immunostaining including, for example, staining of tissues and cells, immunoassays including, for example, competitive immunoassay and non-competitive immunoassay, radioimmunoassay (RIA), FIA, LIA, EIA, ELISA, etc. The detection and measurement (assay) can also be carried with or without B-F separation. In certain embodiments, the detection and measurement is carried out preferably by RIA, EIA, FIA, and LIA, as well as sandwich assay. Incubation is carried out to sequentially react a sample to be assayed, labeled antibodies, and immobilized antibodies. After the non-binding antibodies are separated, the label is detected or measured. The amount of the measured label is proportional to the amount of an antigen, i.e., an E-cadherin antigen. For example, washing, stirring, shaking, filtration, pre-extraction for antigen, etc. is optionally adopted in the measurement or assay process under specific conditions. The other assay conditions including the concentrations of specific reagents, buffers, etc., temperatures, incubation times, and the like can vary according to elements, such as the concentration of antigens in the sample, or the nature of samples to be measured. A person ordinary skilled in the art can suitably select and determine optimal conditions effective for each assay while using the general experimentation and perform the selected measurement.

Various carriers on which antigens or antibodies can be immobilized are available in the art, and they can be arbitrarily and suitably selected for use with the compositions and methods described herein. For immobilization, various carriers which can be used for antigen-antibody interactions are known. Any well-known carrier can be selected and used. Examples are inorganic materials including, for example, glass such as aminoalkylsilyl glass and other activated glass, porous glass, silica gel, silica-alumina, alumina, magnetized iron, magnetized alloy, etc.; organic polymer substances, such as polyethylene, polypropylene, polyvinyl chloride, polyvinylidene fluoride, polyvinyl, polyvinyl acetate, polycarbonate, polymethacrylate, polystyrene, styrene-butadiene copolymer, polyacrylamide, crosslinked polyacrylamide, styrene-methacrylate copolymer, polyglycidyl methacrylate, acrolein-ethylene glycol dimethacrylate copolymer, etc.; cross-linked albumin, collagen, gelatin, dextran, agarose, crosslinked agarose, natural and modified cellulose (for example, cellulose, microcrystalline cellulose, carboxymethylcellulose, cellulose acetate, etc.), crosslinked dextran, polyamides (for example, nylon, etc.), polyurethane, polyepoxy resin, etc.; products obtained by emulsion polymerization of such organic polymer substances; silicon gums, cells, erythrocytes, etc.; and such substances having a functional group introduced thereinto, as required, by using a silane coupling agent, etc.

Also included are solid materials (bodies) such as filter paper, beads, tubes, cuvettes, inner walls of test containers (such as test tubes), titer plates, titer wells, microplates, glass cells, cells made of synthetic materials such as plastic resin cells, glass rods, rods made of synthetic materials, rods thickened or thinned at the end, rods provided with a round protrusion or a flat protrusion at the end, thin-plated rods, and surfaces thereof.

Antibodies and functional derivatives thereof can be coupled with these carriers. Preferably anti-E-cadherin monoclonal antibodies or functional fragments or derivatives of such monoclonal antibodies may be coupled to such a carrier as mentioned above. Coupling between the carrier and those partners associated with these antigen-antibody interactions can be carried out by techniques including physical method such as adsorption; a chemical method using a coupling agent, etc. or an activated reactant; a method using a chemically interactional coupling.

Optionally, the antibody molecules descried herein can be labeled. The label may include an enzyme, enzyme substrate, enzyme inhibitor, prosthetic group, coenzyme, enzyme precursor, apoenzyme, fluorescent substance, pigment, chemiluminescent compound, luminescent substance, coloring substance, magnetic substance, metal particle (such as gold colloids), radioactive substance, and the like. Label enzymes may include dehydrogenases, oxidoreductases such as reductases and oxidases; transferases that catalyze the transfer of functional groups such as amino, carboxyl, methyl, acyl, and phosphate groups; hydrolases that hydrolyze bonds such as ester, glycoside, ether, and peptide bonds; lyases; isomerases; ligases; and the like. Plural enzymes may be used in a conjugated form for detection (for example, enzymatic cycling may also be utilizable). Typical radioactive isotopes for the label include [³²P], [¹²⁵I], [¹³¹I], [³H], [³⁵S], etc. Typical enzymes for the label include peroxidases such as horseradish peroxidase; galactosidases such as E. coli beta-D-galactosidase; maleate dehydrogenases; glucose-6-phosphate dehydrogenases; glucose oxidases; gluocoamylases; acetylcholine esterases; catalases; alkaline phosphatases such as calf intestinal alkaline phosphatase and E. coli alkaline phosphatase, and the like. In the case where alkaline phosphatase is used, measurements can be done by monitoring or inspecting fluorescence, luminescence, etc., generated with a substrate such as umbelliferone derivatives including 4-methylumbellipheryl phosphate; phenol phosphate derivatives including nitrophenyl phosphate; enzymatic cycling systems utilizing NADP; luciferin derivatives; dioxetane derivatives; and the like. It is also possible to use a luciferin/luciferase system. When catalase is used, the reaction takes place with hydrogen peroxide to produce oxygen which can be detected with an electrode or the like. The electrode may be a glass electrode, an ionic electrode using an insoluble salt membrane, a liquid-membrane type electrode, a polymer membrane electrode and the like. The enzyme label may be replaced with a biotin label and an enzyme-labeled avidin (streptavidin). Such use of a biotin-avidin system, use of a secondary antibody, for example, an antibody against an anti-E-cadherin antibody, and other sensitivity-enhancing methods known in the art may be employed. For the label, a plurality of various kinds of labels or markers can be used. In this case, it is possible to perform plural measurements continuously or discontinuously and/or simultaneously or separately.

Signal formation may be done using enzyme-reagent combinations, such as combinations of horseradish peroxidase or other peroxidases with a member selected from 4-hydroxyphenylacetic acid, o-phenylenediamine (OPD), tetramethylbenzidine (TMB), 5-aminosalicylic acid, 3,3-diaminobenzidine tetrahydrochloride (DAB), 3-amino-9-ethylcarbazole (AEC), tyramine, luminol, lucigenin-luciferin or derivatives thereof, Pholad luciferin, etc.; combinations of alkaline phosphatases with a member selected from lumigen PPD, (4-methyl)umbelliferyl phosphate, p-nitrophenol phosphate, phenol phosphate, bromochloroindolyl phosphate (BCIP), AMPAK™ (DAKO), AmpliQ™ (DAKO), etc.; combinations of beta-D-galactosidases or glucose-6-phosphate dehydrogenases with a member selected from 4-methylumbelliferyl-beta-D-galactoside or other umbelliferyl galactosides, o-nitrophenol-beta-D-galactoside, other nitrophenyl galactosides, etc.; combinations of glucose oxidases with ABTS, etc. The signal may be formed with those capable of enzymatically forming quinol compounds such as hydroquinone, hydroxybenzoquinone, and hydroxyanthraquinone, thiol compounds such as lipoic acid and glutathione, phenol derivatives or ferrocene derivatives.

The fluorescent substances and chemiluminescent compounds may include fluorescein isothiocyanate; Rhodamine derivatives such as Rhodamine B isothiocyanate and tetramethyl Rhodamine isothiocyanate (RITC), and tetramethylrhodamine isothiocyanate isomer R (TRITC); 7-amino-4-cumarin-3-acetic acid, dancyl chloride (5-(dimethylamino)-1-naphtalenesulfonyl chloride), dancyl fluoride, fluorescamine (4-phenylspiro[furan-2(3H),1′-(3′H)-isobenzofuran]-3,3′-dione), phycobiliprotein, acridinium salts; luminol compounds such as lumiferin, luciferase and aequorin; imidazoles, oxalic acid esters, rare earth chelate compounds, cumarin derivatives, etc.

Although the generated signals, etc. (including luminescence, fluorescence, etc.) can be detected visually, they may be inspected or monitored with a known device, such as a fluorophotometer and a plate reader. For the detection of signals emitted by a radioactive isotope, etc., a known detector, such as a gamma counter and a scintillation counter, may be used.

The labelling can be accomplished by the reaction of a thiol group with a maleimide group, the reaction of a pyridyldisulfide group with a thiol group, the reaction of an amino group with an aldehyde group, etc. Additionally, it can be suitably selected from widely known methods, techniques which can be easily put into practice by an artisan skilled in the art, and any of modifications derived therefrom.

The coupling agents used for producing the foregoing immunoconjugate or for coupling with carriers are also applicable and usable. The coupling agents include, for example, formaldehyde, glutaraldehyde, hexamethylene diisocyanate, hexamethylene diisothiocyanate, N,N′-polymethylene bisiodoacetamide, N,N′-ethylene bismaleimide, ethylene glycol bissuccinimidyl succinate, bisdiazobenzidine, 1-ethyl-3-(3-dimethylaminopropyl) carbodiimide, succinimidyl 3-(2-pyridyldithio)propionate (SPDP), N-succinimidyl 4-(N-maleimidometyl)cyclohexane-1-carboxylate (SMCC), N-sulfosuccinimidyl 4-(N-maleimidomethyl)-cyclohexane-1-carboxylate, N-succinimidyl (4-iodoacetyl)-aminobenzoate, N-succinimidyl 4-(1-maleimidophenyl)butyrate, N-(epsilon-maleimidocaproyloxy)succinimide (EMCS), iminothiolane, S-acetylmercaptosuccinic anhydride, methyl-3-(4′-dithiopyridyl)propionimidate, methyl-4-mercaptobutyrylimidate, methyl-3-mercaptopropionimidate, N-succinimidyl-5-acetylmercaptoacetate, etc.

According to the assay of the present invention, substances to be measured can be made to react sequentially with labeled antibody reagents (such as antisera, purified antibodies and monoclonal antibodies, labeled with enzymes or the like) and then with antibodies coupled on a carrier, or all the members can be reacted with each other simultaneously. The order of adding reagents (members) may vary depending on the type of carrier system selected. In the case where sensitized beads such as sensitized plastic beads are used, the labeled antibody regents (such as labeled antisera, purified antibodies and monoclonal antibodies) are first put into a suitable test tube, together with a sample including substances to be measured, followed by addition of the sensitized plastic beads. Measurement can be then carried out.

For measurements (and/or detections), an immunological measurement (immunoassay) is applied. For the measurement (assay), the solid phase carriers used may include various materials and shapes which can be selected from balls, microplates, sticks, microparticles, test tubes, and the like, made of polystyrene, polycarbonate, polypropylene, polyvinyl and other materials capable of adsorbing proteins such as antibodies.

The measurement can be carried out in a suitable buffer system so as to maintain an optimal pH (for example, between pH about 4 and about 9). The particularly preferred buffers may include acetate buffers, citrate buffers, phosphate buffers, Tris buffers, triethanolamine buffers, borate buffers, glycine buffers, carbonate buffers, Tris-HCI buffers, veronal buffers, etc. The buffers can be used optionally in a mixed form at any ratio. Preferably, the antigen-antibody interaction is carried out at a temperature between about 0 and 60° C.

The antibody (e.g., antiserum, purified antibody, monoclonal antibody, etc.) reagents labeled with enzymes or others, the immobilized antibody reagents (coupled to a carrier), and substances to be assayed can be incubated until equilibrium is reached. However, the reaction may be stopped after limited incubation wherein the solid phase is separated from the liquid phase at a time point well before the antigen-antibody interaction equilibrates, and the level of labels (such as enzymes) existing in either of the liquid and solid phases may be measured. Measurement operation can be performed with automated measuring instruments.

A luminescence detector, a photo detector or the like may be used to measure or detect indication signals generated as a result of substrate conversion by the action of an enzyme.

Cancer and Cancer Metastasis.

The activating E-cadherin antibodies as provided herein have immediate clinical applications in the treatment and prevention of cancer, and more particularly cancer metastasis. In this context, the terms “cancer”, “cancerous”, “malignant”, and “malignancy” refer to or describe the physiological condition in animals that is typically characterized by unregulated cell growth. The term includes cancers and carcinomas, sarcomas, adenocarcinomas, lymphomas, leukemias, and so forth, including solid tumors and lymphoid cancers, kidney, breast, lung, kidney, bladder, colon, ovarian, prostate, pancreas, stomach, brain, head and neck, skin, uterine, urogenital, testicular, esophagus, and liver cancer, lymphoma (including non-Hodgkin's and Hodgkin's lymphoma), leukemia, and multiple myeloma.

E-cadherin may be under-expressed in cancerous tissue samples from cancer patients where the cancers are likely to become invasive, to metastasize, or to progress or become treatment refractory. This under-expression may be two-fold, three-fold, four-fold, or five-fold or greater. Such differences may be readily apparent through any recognized form of quantification, whether or not quantitative measurements are used, including for instance, when viewing the bands of gels with approximately similarly loaded with test and control samples.

The cancer to be treated herein may be one characterized by insufficient activation of E-cadherin. Alternatively, the cancer to be treated herein may be one where the E-cadherin protein is expressed at normal or low levels, or one where the E-cadherin protein is expressed by a subset of cells, and where the E-cadherin protein is not overexpressed. In one embodiment of the invention, a diagnostic or prognostic assay is performed to determine whether the patient's cancer is characterized by expression of E-cadherin. Various assays for determining such amplification/express ion are contemplated and include the immunohistochemistry, FISH and shed antigen assays, southern blotting, or PCR techniques. Moreover, the E-cadherin expression or amplification may be evaluated using an in vivo diagnostic assay, e.g. by administering a molecule (such as an antibody) which binds the molecule to be detected and is tagged with a detectable label (e.g. a radioactive isotope) and externally scanning the patient for localization of the label. In some embodiments, the cancer to be treated is not yet invasive, but expresses E-cadherin. The terms “cancer that expresses E-Cadherin” and “cancer associated with the expression of E-Cadherin” interchangeably refer to cancer cells or tissues that express E-Cadherin.

As cancers progress, they may metastasize. The term “metastasis” or “metastatic disease”, as used herein, refers to the spread of a cancer or disease from one organ or part in a body to another not directly connected with it. Metastasis, the process whereby tumor cells migrate throughout the body, is complex. In order for a tumor to produce metastases it must contain cells of the correct genotype be capable of completing a complex series of steps. The steps of tumor cell metastasis include the detachment of tumor cells from the primary neoplasm, invasion into the surrounding stroma, intravasation into the vasculature or lymphatic system, survival in the circulation, extravasation into the new host organ or tissue, and then survival and growth in this new microenvironment. When tumor cells metastasize, the new tumor is called a secondary or metastatic tumor, and its cells are similar to those in the original tumor. See also U.S. 2009/00023143.

In specific method embodiments, there are contemplated treatment methods intended to treat, prevent, or ameliorate metastasis of a tumor in a subject wherein an anti-E-cadherin antibody molecule is administered to the subject after a carcinoma or other epithelial-derived cancer has been diagnosed. Optionally such administration occurs before an metastatic event has been diagnosed in the subject. In some embodiments, it is contemplated that such treatment is carried out (and/or initiated) after a metastatic event has been identified. It is also contemplated that the antibodies provided herein can be used to treat a subject after a carcinoma has been both diagnosed and treated, for instance through surgical removal, chemotherapy, radiation therapy, and/or any other art-recognized means of cancer therapy appropriate for that carcinoma. Specifically, it is proposed that the activating E-cadherin specific antibodies provided herein will be useful to reduce or block the spread of cancerous cells (that is, metastatic cells) to other sites remote from the initial tumor.

Examples of cancer that can be treated using the activating E-cadherin antibody molecules provided herein include any cancers that involve an epithelial-origin tissue or cell. These include particularly carcinomas (defined as malignancies of epithelial tissues), which encompasses as much as 85% of diagnosed cancers and includes the two general subtypes: adenocarcinomas and squamous cell carcinomas. Also included are transition cell carcinomas and basal cell carcinomas. More particular examples of cancers that can be treated using the compositions and methods provided herein include small-cell lung cancer, non-small cell lung cancer, gastrointestinal (tract) cancer, renal cancer, kidney cancer, liver cancer, stomach cancer, gastric cancer, colon carcinoma, colorectal cancer, ovarian cancer, cervical cancer, endometrial cancer, prostate cancer, melanoma, pancreatic cancer, bladder cancer, hepatoma, and breast cancer. The following table provides additional categorization of representative tumors (benign and malignant) that develop from eoithelial tissues.

TABLE 7

| | Representative | Representative |
|---|---|---|
| Tissue | Benign Tumors | Malignant Tumors |
| Stratified | Papilloma, Seborrheic | Squamous cell carcinoma; |
| squamous | keratosis and some | epidermoid carcinoma, and |
| | skin adnexal tumors | malignant skin adnexal tumors |
| Glandular | Adenoma | Adenocarcinoma |
| epithelium | Hepatic adenoma | Hepatoma (hepatocellular |
| (Liver, Kidney, | Renal tubular | carcinoma) |
| Bile duct) | adenoma | Renal cell carcinoma; |
| | Bile duct adenoma | hypernephroma; |
| | | Cholangiocarcinoma |
| Transitional | Transitional cell | Transitional cell carcinoma |
| epithelium | papilloma | |
| Placenta | Hydatidiform mole | Choriocarcinoma |
| Testis | N/A | Seminoma; embryonal |
| | | cell carcinoma |

Diseases & Conditions with Defective or Disrupted Epithelial Barrier Function

Also provided herein are new approaches to target and treat diseases and conditions influenced by defective or disrupted epithelial barrier function (such as inflammatory bowel disease (IBD) and conditions impacting respiratory system permeability, including the attendant inflammatory aspects of such diseases/conditions). These approaches involve preventing or treating disease by enhancing barrier function, through activation of E-cadherin. Described herein are ways to activate cell-cell adhesion and cell-cell junction formation, using activating monoclonal antibodies to E-cadherin (such as mAbs 19A11, 66E8, 56-4, 18-5, and humanized versions and functional fragments thereof). These mAbs are used to target skin, gastrointestinal, and/or airway epithelium rather than immune cell compartment.

The intestinal epithelium (when healthy) separates microbes, immunogens, and toxins in intestine contents from the immune/inflammatory compartment. Increased intestinal permeability is associated with IBD—it precedes overt disease (demonstrated in both human and mice) and exhibits detectable familial association in humans. Modulation of GI epithelial barrier function is controlled by the activity state of E-cadherin at the cell surface and p120-catenin phosphorylation. IBD-associated and/or inflammatory processes downregulate E-cadherin adhesive activity.

Since GI barrier permeability had been strongly implicated in the etiology of IBD, it is proposed that the E-cadherin activating mAbs described herein, as well as p120-catenin mutations, can be used to enhance GI barrier function and slow the progression of IBD. Activating E-cadherin (for instance, using the activating mAbs described herein) will enhance GI barrier function and thereby reduce inflammatory responses and the progression of IBD. Also contemplated is use of activating mutations in p120-catenin (a cadherin associated protein) to influence intestinal epithelium permeability. In particular embodiments, activating mutations in p120-catenin include S252A, S268A, S288A, T310A, and S312A described in Petrova et al. Molecular Biology of the Cell 23(11): 2092-2108, 2012. These mutations create a p120-catenin that is non-phosphorylatable, leading to increased adhesion in cells containing these mutations.

IBD encompasses disorders that involve chronic inflammation of the digestive tract. In particular embodiments, IBD includes ulcerative colitis (UC) and Crohn's disease. Symptoms common to both UC and Crohn's disease include: diarrhea, fever and fatigue, abdominal pain and cramping, bloody stools, reduced appetite, and weight loss. UC occurs in the large intestine (colon) and the rectum. Damage in UC is continuous (not patchy), and inflammation is present only in the innermost layer of the lining of the colon. Crohn's disease can affect any part of the GI tract, including from the mouth to the anus. In particular embodiments, Crohn's disease can affect the portion of the small intestine before the colon. Damaged areas in Crohn's disease are patchy and appear next to areas of healthy tissue, and inflammation may reach through multiple layers of the walls of the GI tract. IBD can be diagnosed using endoscopy and/or colonoscopy and imaging tools, including contrast radiography, magnetic resonance imaging (MRI), and/or computed tomography (CT). Stool samples may also be checked. IBD can currently be treated with medications including: aminosalicylates, corticosteroids (e.g., prednisone), immunomodulators, and biologics. Surgery can be used to remove damaged portions of the GI tract.

Similarly, the airway epithelium serves as a barrier to protect underlying tissues from microbial contamination as well as environmental insult. The integrity of the airway epithelium is maintained by ultrastructural components that connect neighboring epithelial cells such as: tight junctions (zonula occludens, ZO), adherens junctions, desmosomes (macula adherens), and hemidesmosomes. Tight junctions separate the apical and basolateral regions, and at least 40 different proteins have been identified as tight junction components. Adherens junctions create an intercellular space of 25-35 nm and are located directly beneath the tight junctions. Adherens junctions include two adhesive parts, the nectin-afadin complex and the E-cadherin-catenin complex. Desmosomes are cell-cell adhesion structures mediated by E-cadherin and provide mechanical support to a tissue. Desmosomes can establish adhesive connections of columnar epithelial cells to the basement membrane or of columnar epithelial cells to basal cells. Hemidesmosomes, which primarily include integrins, connect basal cells to the extracellular matrix in the basement membrane. Disruption of that barrier is seen in myriad conditions, including allergic airway inflammation, asthma, chronic obstructive pulmonary disease (COPD), and bronchopulmonary dysplasia (BPD). In particular embodiments, signs of epithelial damage can include: increased respiratory epithelial barrier permeability; destruction or reduction of cell-cell junction components; higher sensitivity to oxidants; overproduction of mucus; and inadequate innate immune response to bacterial and/or viral infections in the respiratory tract. See, for instance: Xiao et al. (J. Allergy Clin Immunol. 128(3):549-556, 2011), Georas & Rezaee (J. Allergy Clin Immunol 134(3)503-520, 2014), Yuksel & Turkeli (Tissue Barriers. 5(4):e1367458, 2017), Schleimer & Berdnikovs, J Allergy Clin Immunol 139(3):1752-1761, 2018. In particular embodiments, airway inflammation diseases or disorders include acute respiratory distress syndrome (ARDS), COPD, asthma, cystic fibrosis, allergic rhinitis, chronic rhinosinusitis, and bronchiolitis.

The art recognizes many other disease and conditions that are associated with defective or disrupted epithelia barrier function, in the skin, gastrointestinal tract, or airway. See Schleimer & Berdnikovs, J Allergy Clin Immunol 139(3):1752-1761, 2018, which discusses epithelial barrier dysfunction particularly in diverse Type 2 inflammatory diseases, for instance. It is proposed that treatment with the activating anti-E-cadherin antibody molecules described herein will be useful for treating these conditions. In particular embodiments, inflammatory disorders that can be treated with E-cadherin activating monoclonal antibodies of the present disclosure include: atopic dermatitis, asthma, allergic rhinitis, chronic rhinosinusitis, and eosinophilic esophagitis.

In particular embodiments, inflammatory disorders that can be treated with E-cadherin activating monoclonal antibodies of the present disclosure include autoimmune diseases or disorders. In particular embodiments, autoimmune diseases or disorders include: type I diabetes, multiple sclerosis, systemic lupus erythematosus, rheumatoid arthritis, and psoriasis. In particular embodiments, autoimmune diseases or disorders that can be treated with E-cadherin activating monoclonal antibodies of the present disclosure are associated with altered intestinal microbiota composition.

The activating E-cadherin antibodies as provided herein have immediate clinical applications in the treatment and prevention of Acute Respiratory Distress Syndrome (ARDS). ARDS is a severe, and often life-threatening complication of several systemic disorders and direct injury to the lungs, involving acute lung inflammation, non-cardiogenic pulmonary edema, and acute respiratory failure. It is associated with a high mortality rate, primarily as a consequence of multiple organ failure (Frutos-Vivar et al., Curr Opin Crit Care 10:1-6, 2004). ARDS can include mild, moderate, and severe categories. In particular embodiments, ARDS can include acute hypoxemia, a ratio of the PaO2 to the FiO2 of 300 mmHg or less on positive end-expiratory pressure (PEEP) of 5 cm H₂O or greater, together with bilateral infiltration on radiology that is not otherwise fully explained by fluid overload or cardiac failure (ARDS Definition Task Force; Ranieri et al. JAMA 307(23): 2526-2533, 2012). Normal gas exchange in the lungs is enabled by the tight barrier that is usually formed between atmosphere and fluid-filled tissue. The normal alveolar barrier is composed of three different structures: (1) the capillary endothelium, (2) an interstitial space including a basement membrane and extracellular matrix, and (3) the alveolar epithelium. The alveolar epithelium includes alveolar type I and alveolar type II cells. Alveolar type I cells are flat, line more than 90% of the alveolar surface area, and provide structure for optimal exchange of respiratory gases between the alveolar lumen and bloodstream. The cuboidal alveolar type II cells have multiple functions: producing surfactant, a naturally-produced foamy substance that keeps the lungs fully expanded for breathing; clearing active alveolar liquid; and serving as progenitor cells for regeneration of the alveolar epithelium after injury. Under normal conditions the epithelial barrier is much less permeable than the endothelial barrier and prevents cells and plasma from flooding the air spaces, thereby maintaining normal gas exchange (Geiser, T. Swiss medical weekly 133(43/44): 586-590, 2003). Disruption and failure of the endothelial-epithelial barrier result in devastating consequences, leading to alveolar flooding and subsequent fibrotic scarring in the lungs. ARDS occurs when fluid builds up in the alveoli of the lungs and surfactant breaks down. The fluid buildup and surfactant breakdown prevent the lungs from filling properly with air and moving enough oxygen into the bloodstream, leading to deprivation of oxygen in organs throughout the body. In particular embodiments, activating E-cadherin antibodies of the present disclosure can treat or prevent ARDS by reducing or preventing alveolar epithelial barrier disruption. In particular embodiments, activating E-cadherin antibodies of the present disclosure do not directly reduce or prevent endothelial barrier disruption.

ARDS typically occurs in people who are already critically ill or who have significant injuries. Injuries or clinical conditions that are associated with ARDS include: acute lung injury (ALI); sepsis and/or systemic inflammatory response syndrome (SIRS) (life-threatening conditions that occurs when the immune system must work aggressively to fight off infection or trauma); inhalation of harmful substances; severe pulmonary infection (e.g., pneumonia); severe traumatic injury (e.g., multiple fractures); severe head injury; pulmonary contusion; blood transfusions; pancreatitis; overdoses of narcotics; and near drowning. In particular embodiments, viruses such as influenza can damage the alveolar barrier (Short et al. European Respiratory Journal 47: 954-966, 2016). Symptoms of ARDS includes severe shortness of breath, labored and unusually rapid breathing, low blood pressure, and confusion and extreme tiredness, which usually develops within a few hours to a few days after an original disease or trauma. The risk of death increases with age and severity of illness. Of those that survive ARDS, some recover completely while others experience lasting damage to their lungs. Patients with ARDS can develop other complications, including blood clots, collapsed lung, infections, and pulmonary fibrosis (scarring and thickening of the tissue between the air sacs).

(V) Kits

Active component(s), including particularly at least one described engineered E-cadherin activating antibody or biologically active fragment thereof, can be provided as kits. Kits can include one or more containers including (containing) one or more or more compounds as described herein, optionally along with one or more agents for use in therapy. For instance, some kits will include an amount of at least one additional anti-cancer composition, or an amount of at least one additional anti-inflammatory agent, or both.

Any active component in a kit may be provided in premeasured dosages, though this is not required; and it is anticipated that certain kits will include more than one dose.

Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding administration; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary medical supplies needed to use the kit effectively, such as applicators, ampules, sponges, sterile adhesive strips, Chloraprep, gloves, and the like. Variations in contents of any of the kits described herein can be made. The instructions of the kit will direct use of the active ingredient(s) included in that kit to effectuate a clinical and/or therapeutic use described herein.

Suitable methods, materials, and examples used in the practice and/or testing of embodiments of the disclosed invention are described herein. Such methods and materials are illustrative only and are not intended to be limiting. Other methods, materials, and examples similar or equivalent to those described herein can be used.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

(VI) Exemplary Embodiments.

1. An engineered antibody including: the heavy chain CDR1, CDR2 and CDR3 shown, respectively, in SEQ ID NO: 17, 18, and 19, and the light chain CDR1, CDR2 and CDR3 shown, respectively, in SEQ ID NO: 20, 21, and 22; or the heavy chain CDR1, CDR2 and CDR3 shown, respectively, in SEQ ID NO: 23, 24, and 25, and the light chain CDR1, CDR2 and CDR3 shown, respectively, in SEQ ID NO: 26, 27, and 28; or the heavy chain CDR1, CDR2 and CDR3 shown, respectively, in SEQ ID NO: 29, 30, and 31, and the light chain CDR1, CDR2 and CDR3 shown, respectively, in SEQ ID NO: 32, 33, and 34; or the heavy chain CDR1, CDR2 and CDR3 shown, respectively, in SEQ ID NO: 35, 36, and 37, and the light chain CDR1, CDR2 and CDR3 shown, respectively, in SEQ ID NO: 38, 39, and 40.
2. The engineered antibody of embodiment 1, which is a humanized antibody.
3. The engineered antibody of embodiment 1, which is a Fab, an IgG, a scFv, a diabody, or bispecific antibody.
4. The engineered antibody of embodiment 1, which binds specifically to and activates E-cadherin.
5. An engineered antibody that binds specifically to and activates E-cadherin, including: the heavy chain variable (VH) domain shown in SEQ ID NO: 2 and the light chain variable (VL) domain shown in SEQ ID NO: 4; or the VH domain shown in SEQ ID NO: 6 and the VL domain having SEQ ID NO: 8; or the VH domain shown in SEQ ID NO: 10 and the VL shown in SEQ ID NO: 12; or the VH domain shown in SEQ ID NO: 14 and the VL domain shown in SEQ ID NO: 16.
6. The engineered antibody of embodiment 5, including:

the VH domain shown in SEQ ID NO: 2 and the VL domain shown in SEQ ID NO: 4.

7. The engineered antibody of embodiment 5, including: the VH domain shown in SEQ ID NO: 6 and the VL domain having SEQ ID NO: 8.
8. The engineered antibody of embodiment 5, including: the VH domain shown in SEQ ID NO: 10 and the VL domain shown in SEQ ID NO: 12.
9. The engineered antibody of embodiment 5, including: the VH domain shown in SEQ ID NO: 14 and the VL domain shown in SEQ ID NO: 16.
10. An engineered antibody including the monoclonal antibody 19A11, 66E8, 56-4, or 18-5, or a humanized version or functional fragment thereof.
11. A polynucleotide encoding the antibody of embodiment 5, wherein the polynucleotide includes: the polynucleotide sequence encoding the VH domain shown in SEQ ID NO: 1; or the polynucleotide sequence encoding the VL domain that is shown in SEQ ID NO: 3; or both.
12. A polynucleotide encoding the antibody of embodiment 5, wherein the polynucleotide includes: the polynucleotide sequence encoding the VH domain shown in SEQ ID NO: 5; or the polynucleotide sequence encoding the VL domain that is shown in SEQ ID NO: 7; or both.
13. A polynucleotide encoding the antibody of embodiment 5, wherein the polynucleotide includes: the polynucleotide sequence encoding the VH domain shown in SEQ ID NO: 9; or the polynucleotide sequence encoding the VL domain that is shown in SEQ ID NO: 11; or both.
14. A polynucleotide encoding the antibody of embodiment 5, wherein the polynucleotide includes: the polynucleotide sequence encoding the VH domain shown in SEQ ID NO: 13; or the polynucleotide sequence encoding the VL domain that is shown in SEQ ID NO: 15; or both.
15. Use of the antibody of any one of embodiments 1-10 or encoded by the polynucleotide of any of embodiments 11-14 that specifically binds to and activates human E-cadherin to treat, prevent, or ameliorate: cancer metastasis; inflammatory bowel disease; or airway inflammation; or use to treat, prevent, or ameliorate any disease or condition associated with or involving defective or disrupted epithelial barrier function.
16. The use of embodiment 15, wherein the airway inflammation includes acute respiratory distress syndrome (ARDS).
17. A method for treating cancer in a subject, including: administering to a subject in need of such treatment a therapeutically effective amount of the engineered antibody of any one of embodiments 1-10 or encoded by the polynucleotide of any of embodiments 11-14 that specifically binds to and activates human E-cadherin.
18. The method of embodiment 17, wherein treating cancer includes reducing cancer metastasis.
19. A method of treating a cancer patient with a cancer that expresses an E-cadherin protein, including: obtaining a tissue sample from an individual at risk of having a cancer that expresses an E-cadherin protein; determining the presence or absence or amount of the E-cadherin protein in the tissue sample in comparison to a control tissue sample from an individual known to be negative for the cancer; thereby diagnosing the individual at risk as a cancer patient with a cancer that expresses an E-cadherin protein, wherein the E-cadherin protein is expressed at normal or low levels, or is expressed by a subset of cells, or is overexpressed; and administering to the cancer patient with a cancer that expresses an E-cadherin protein a therapeutically effective amount of the engineered antibody of any one of embodiments 1-10 or encoded by the polynucleotide of any of embodiments 11-14, or an antigen-binding antibody fragment thereof, that specifically binds to and activates human E-cadherin.
20. A method for treating a subject having an inflammatory disorder including: administering to a subject in need of such treatment a therapeutically effective amount of the engineered antibody of any one of embodiments 1-10 or encoded by the polynucleotide of any of embodiments 11-14 that specifically binds to and activates human E-cadherin.
21. The method of embodiment 20, wherein the inflammatory disorder includes inflammatory bowel disease or airway inflammation.
22. The method of embodiment 21, wherein the airway inflammation includes acute respiratory distress syndrome (ARDS).
23. The method of embodiment 20, wherein the inflammatory disorder includes an autoimmune disease.
24. The method of embodiment 20, wherein the inflammatory disorder is characterized by disruption of normal cell adhesion and/or cell junctions.
25. The method of embodiment 20, wherein the engineered antibody is administered locally to a site of inflammation in the subject.
26. The method of embodiment 20, wherein the engineered antibody includes monoclonal antibody 19A11, 66E8, or a humanized version or functional fragment thereof.
27. A method for modulating cell adhesion of E-cadherin-expressing cells including: contacting the cells with the engineered antibody of any one of embodiments 1-10 or encoded by the polynucleotide of any of embodiments 11-14.

### Example 1: Production and Sequencing of E-cadherin Antibodies

This example describes the production of the E-cadherin antibodies used in other examples.

Generation of activating rabbit mAbs to mouse E-cadherin. Hybridoma cell lines were generated from rabbits immunized with the purified extracellular domain of mouse E-cadherin by Epitomics (Burlingame, Calif.), which is now owned by AbCam (Cambridge, Mass.). E-cadherin hybridomas positive for E-cadherin binding in ELISA were then screened in a functional assay as done previously for mouse anti-human E-cadherin, for their ability to activate adhesion in colo205 cells (Petrova et al., Mol. Biol. Cell. 23:2092-2108, 2012). In this case, colo205 cells expressing mouse E-cadherin were used after knocking down endogenous human E-cadherin expression with an shRNA. Hybridomas producing activating mAbs 18-5, 56-4 were obtained; also, a hybridoma line producing a neutral antibody 19.1-10 was obtained that binds E-cadherin but does not activate colo205 adhesion.

Generation of recombinant antibodies. Hybridoma cell lines producing mAbs to both mouse anti-human E-cadherin (Petrova et al., Mol. Biol. Cell. 23:2092-2108, 2012; 19A11, 66E8, 46H7) and Rabbit anti-mouse E-cadherin (18-5, 56-4, 19.1-10) were sent to GenScript (Piscataway, N.J.) to sequence the variable regions of the heavy and light chains for each mAb. Total RNA was isolated from the hybridoma cells following the technical manual of TRIzol® Reagent. Total RNA was then reverse-transcribed into cDNA using either isotype-specific anti-sense primers or universal primers following the technical manual of PrimeScript™ 1st Strand cDNA Synthesis Kit. Antibody fragments of VH, VL, CH and CL were amplified according to the standard operating procedure (SOP) of rapid amplification of cDNA ends (RACE) of GenScript. Amplified antibody fragments were cloned into a standard cloning vector separately. Colony PCR was performed to screen for clones with inserts of correct sizes. No less than five colonies with inserts of correct sizes were sequenced for each fragment. The sequences of different clones were aligned and the consensus sequence was provided.

The sequenced heavy and light chains for each mAb were all then cloned into the backbone of mouse IgG1 constant region encoding sequences. The full heavy chain and light chain sequences were then cloned into pcDNA3.4 and expressed in ExpiCHO cells (Invitrogen) following the manufacturer's protocols. Two weeks post transfection, antibodies were affinity purified from about 350 mLs of media on a 5mL protein G column (HiTrap MabSelect SuRe, GE Healthcare Life Sciences, Pittsburgh, Pa.), buffer-exchanged to PBS pH7.2 and stored as sterile aliquots at −80° until use.

The sequences of the variable heavy and light chains for each of 19A11, 66E8, 56-4, and 18-5 antibodies were determined, and are provided in SEQ ID NOs: 1-16. The CDRs for each are provided in SEQ ID NOs: 17-40, as described in Tables 1 and 3.

### Example 2: E-cadherin Activity Affects Multiple Cell Mechanisms in Breast Cancer Metastases

This Example describes the importance of E-cadherin activation that can inhibit the overall process of metastatic cascades, including cell-cell adhesion, local invasion, intravasation, dissemination, extravasation, and colonization at the target organ. At least some of the results presented in this Example were published in Na et al. (PNAS 117(11):5931-5937, 2020).

E-cadherin is a well-known tumor suppressor protein and the loss of its expression in tumor cells occurs frequently during tumor progression and metastasis. However, loss of E-cadherin expression is an oversimplification as many metastases still contain high levels of E-cadherin and epithelial cells expressing E-cadherin can become invasive and/or undergo an EMT-like process and metastasis.

First, it is demonstrated that the expression of E-cadherin was still present in metastatic lung nodules and circulating tumor cells (CTCs) of MMTV-PyMT mice and 4T1 tumor cell-inoculated mice, but metastasis occurred strongly. Importantly, treatment of mAbs significantly delayed lung metastasis and reduced the number of CTCs regardless of the expression of E-cadherin. Also, it was found that E-cadherin activation can inhibit extravasation in the induced vasculature by injecting 4T1 cell expressing hE-cadherin into the tail vein. Second, 3D organoids or in vitro cell cultures shown that the suppression of tumor metastasis by treatment of mAbs was due to inducing cell attachment and reducing cell migration/invasion. In addition, in vitro and in vivo treatment of mAbs increased TUNEL− and Cleaved caspase3 positive cell but no change was observed in proliferation. Finally, the effect of mAbs on apoptosis was confirmed in circulating tumor cells sorted in whole blood cells. In the treatment of activating antibodies, anti-apoptotic marker was reduced and pro-apoptotic marker showed an increased pattern after 10 days of 4T1 tumor cell transplantation, indicating that monoclonal antibody can increase sensitivity to the entire cell apoptosis. The results described here suggest that activating monoclonal antibody (mAb) to E-cadherin that induces a high adhesive state can control the metastatic cascade of multi-step process involving tumor cell dissemination, intravasation, and extravasation by regulation of cell-cell adhesion, migration/invasion and apoptosis.

This study tested whether E-cadherin activation by mAbs can affect multi-step process which includes regulation of tumor cell dissemination by cell-cell adhesion, local tumor cell invasion, entry into the vasculature followed by the exit of carcinoma cells from the circulation and colonization at the distal sites. Based on the reported results, E-cadherin activation may provide a novel therapeutic strategy to delay metastasis in breast cancer patients with high level of E-cadherin.

Introduction: Traditional understanding of epithelial cancer metastasis is derived primarily from mouse models and it is thought to involve a series of sequential steps: Epithelial to mesenchymal transition (EMT) of individual cells within the primary tumor leading to their intravasation into the bloodstream, survival of such circulating tumor cells (CTCs) within the bloodstream, and final their extravasation at distant sites, where mesenchymal-epithelial transition (MET) culminates in their proliferation as epithelial metastatic deposits. In light of its well-established function in maintaining adherens junctions, loss of E-cadherin epithelial cell adhesion protein ostensibly promotes metastasis by enabling the first step of the metastatic cascade: the disaggregation of cancer cells from one another. In addition, loss of the E-cadherin expression has been long considered to increase tumor cell invasiveness in vitro and contributes to the transition of adenoma to carcinoma in animal models. However, loss of E-cadherin expression is an oversimplification as many metastases still contain high levels of E-cadherin and epithelial cells expressing E-cadherin can become invasive and/or undergo an EMT-like process and metastasize in various cancers. Also, whether the loss of E-cadherin expression has successfully completed the various stages of the invasion-metastasis cascade is unclear. In fact, invasive leader cells in primary breast tumor and circulating tumor cell clusters in the blood still maintained expression of E-cadherin and E-cadherin is involved in collective cell behaviors that facilitate invasion and metastasis.

E-cadherin is a well-known important metastasis suppressor, but the regulation of its state of activation is almost unknown. Previous work on both Xenopus C-cadherin and human E-cadherin provided evidence for the regulation of cadherin adhesion activity independent of cell surface expression levels. Physiological regulation of C-cadherin in response to growth factors during embryonic morphogenesis involves changes in the adhesive state of cadherins at the cell surface, without changes in either expression levels at the cell surface or amounts of associated catenins. Furthermore, E-cadherin adhesive activity can be regulated at the cell surface by an inside-out signaling mechanism probably involving allosteric regulation of the homophilic adhesive bond, analogous to integrin regulation.

Based on these multiple perspectives, specific monoclonal antibodies (mAbs) have been developed that can bind to E-cadherin and distinguish the activity and inactivity of E-cadherin from the cell surface. E-cadherin adhesive activity is dynamically regulated at the cell surface in tumor cells and an activating monoclonal antibody to E-cadherin that induces a high adhesive state significantly decreased the number of cells metastasized to the distal organ without affecting the growth in size of primary tumor in the mammary gland. This indicates that low activity of E-cadherin on the surface of tumor cells is important for metastasis and that activation of its function with mAbs can suppress metastasis.

The resulting loss of cell-cell adhesion and cell junctions mediated by E-cadherin homophilic binding is believed to allow cells to dissociate from the primary tumor, invade surrounding tissues, and migrate to distant sites. The simplest and longest held idea about the role of E-cadherin in metastasis is that it prevents the initial dissociation of epithelial cells from the original tumor mass. However, it has been found that dynamic regulation of E-cadherin is required for collective cell migration, in normal development and tumor growth. Also, E-cadherin reduces proliferation through contact inhibition and signaling, and even has been claimed to stimulate proliferation in some contexts. Therefore, it is important to determine the stage of metastatic progression affected by E-cadherin activation. This study tested whether E-cadherin activation by mAbs can affect multi-step process which includes regulation of tumor cell dissemination by cell-cell adhesion, local tumor cell invasion, entry into the vasculature followed by the exit of carcinoma cells from the circulation and colonization at the distal sites. Here, it is proposed that E-cadherin activation provides a novel therapeutic strategy to delay metastasis in breast cancer patients with high level of E-cadherin.

Materials and Methods

Animal studies and in vivo treatments of antibodies: FVB MMTV-Polymavirus middle T antigen (MMTV-PyMT) breeders were obtained from The Jackson Laboratory and mated according to the vendor's specifications. All mice were housed and bred under specific pathogen-free conditions at Seattle Children's Research Institute (SCRI) and all animal studies are governed through protocols approved by the Institutional Animal Care and Use Committee (IACUC).

Four-week-old transgenic mice were treated twice weekly with neutral mAbs, or E-cadherin-specific mAbs (5 mg/kg of weight) in saline by intraperitoneal injection. Caliper measurements of primary tumor were done weekly until the end of the experiments. Tissues of 14-week-old mouse were isolated and fixed in Bouin's solution and whole blood were harvested. 4T1-Luc2-hE (4T1-hE) cells suspended in lx HBSS were injected into the mammary fat pads (1×10⁴) of BALB/c mice. To determine the ability of tumor cells to colonize the lung, 4T1-hE cells (3×10⁴) were also injected via the tail vein. The generation of 4T1-hE cell line and animal experiment of 4T1-hE cells was described previously (Petrova et al. Mol Biol Cell 27:3233-3244, 2016).

New Antibodies: E-Cadherin antibodies were produced as described in Example 1.

Histological analysis, immunofluorescence staining, and detection of apoptosis in tumors: Paraffin section (5 μm) were stained with hematoxylin and eosin (H&E, Sigma-Aldrich, St. Louis, Mo., USA). For immunofluorescence staining, the tissue sections were hydrated in a series of washes from xylenes to ethanol dilutions to water. Heat-mediated antigen retrieval was conducted with citrate buffer. Endogenous peroxidase activity was blocked with 3% hydrogen peroxide. Samples were blocked with 5% bovine serum albumin (BSA) (Sigma-Aldrich) and primary antibodies were incubated at 4° C. overnight in 1% BSA in PBS for the following antibodies at indicated concentrations: E-cadherin (BD Biosciences, 610181) 1:1,000; cleaved-caspase 3 (Cell Signaling, 9661) 1:500; pHistone3 (Cell Signaling, 9701) 1:500. Alexa Fluor-conjugated secondary antibodies were incubated on tissues for 1 hour (1:1,000). Hoechst was used to stain the nuclei. For cells, 4T1 and MCF10a cells were fixed with 4% paraformaldehyde and incubated with cleaved caspase-3 antibody at 4° C. overnight in 1% BSA in PBS. The coverslips were mounted on glass slides and the slides were imaged using Leica DFC310 FX and Olympus IX71 microscope. For determination of apoptosis in tumor tissues and cell culture, terminal deoxynucleotidyltransferase-mediated dNTP nick end—labeling (TUNEL) assay was performed using the ApopTag® Red In Situ Apoptosis Detection kit (S7165, Millipore).

Isolation of circulating tumor cell and circulating tumor cell (CTC) detection using qRT-PCR and flow cytometry: 900 μI of blood was obtained by cardiac puncture from mouse and processed according to standard separation protocols. After centrifugation, the buffy coat is collected for isolation of CTCs and lysis of any red blood cells collected with the buffy coat is ensured using red blood cell lysis buffer. To count the estimated number of circulating cancer cells, mRNA levels of epithelial markers, or PyMT and luciferase expression, were measured. Putative number of CTCs were calculated from equation for the mRNA levels in cultured Py2T or 4T1-Luc2-hE cells. For accurate analysis of apoptosis markers in circulating blood cells, only the cells expressing the epithelial marker were sorted using a BD FACSAria I (BD Biosciences) and the mRNA was measured by qRT-PCR. CTCs were washed and suspended in 500 pL containing ice-cold Hank's Balanced Salt Solution (HBSS) containing 0.5% BSA. Cells were stained with 1 μg/ml 19A11 and fluorescein isothiocyanate-conjugated secondary antibody (1:500) in ice-cold HBSS containing 5% FBS. The antibodies were incubated for 45 minutes and washed to remove the excess of antibodies. The cytometric analysis and cell sorting were carried out using a FACS Aria-II flow cytometer (BD Biosciences, San Jose, Calif., USA). Cells were kept in sterile conditions during cell sorting and flow cytometric analysis of membrane E-cadherin protein expression was performed before cell sorting. The Flow Jo software was used for data acquisition and analysis. FACS-purified cell purity was verified by qPCR analysis using the epithelial markers and CD45 as a panleukocyte marker was not detected. Total RNA was prepared from FACS-sorted circulating tumor cells or cell lines using TRIzol (Life Technologies). RNA purity was confirmed using a NanoDrop Spectrophotometer (Thermo Fisher Scientific). The RNA samples were reverse-transcribed using a first-strand cDNA synthesis kit (SensiFAST™ cDNA Synthesis Kit) and subjected to qPCR (ΔΔCT) analysis, using KAPA SYBR® FAST qPCR Kits (Kapa Biosystems). cDNA samples contained at least 10 ng/μl. Forward and reverse primer sets were as shown in SEQ ID NOs: 41-60.

Isolation and 3D culture of primary murine mammary organoid: Epithelial fragments termed organoids were isolated from murine mammary tumor. MMTV-PyMT tumors were harvested from mice at 14 wks of age. No. 3 and no. 4 mammary glands were dissected and digested into epithelial fragments by a combination of mechanical disruption and collagenase/trypsin digestion, and DNase treatment to separate epithelial tissue from fat and stromal cells. Briefly, they were minced into small fragments using a sterile razor blade and were incubated (typically in 10 mL of solution in a 15-mL Falcon tube) in collagenase [high-glucose DMEM (D6546; Sigma), 2 mM glutamine (5.1 mL), 200 U/mL penicillin/200 μg/mL streptomycin and 2 mg/mL collagenase I (C2139; Sigma) ] with rocking at 37° C. Successful isolation and culture were achieved with incubation times ranging from 6 h to overnight and in digestion solutions of collagenase alone or collagenase plus trypsin. The epithelial fragments were separated from single cells through differential centrifugation. The final pellet was composed of epithelial fragments, each containing several hundred cells; these fragments are term “organoids”. Spheroids are generated in drops of media that hang from the lid of a tissue culture dish for 72 hr. Next, the drops are pooled, and the spheroids are transferred to a 4° C. mixture of basement membrane materials (1:1 ratio of Matrigel (354230; BD Biosciences) and Collagen, Type 1 solution from rat tail (C3867-1VL; Sigma-Aldrich)). Following spheroid resuspension, the viscous mixture is pipetted into the wells of a 24 well plate or 8-well Lab-Tek chamber slides (Nalgene-Nunc/Thermofisher Scientific), after which it is given 30 min at 37° C. to solidify into a 3D culture. Warm media containing mAbs is then added to the wells. Organoid medium: DMEM (Sigma D6546), 2 mM glutamine (ATCC or Invitrogen), 100 U/mL penicillin/100 μg/mL streptomycin, 10 mM Hepes (H3375-250g; Sigma), 0.075% (wt/vol) BSA (A8412; Sigma), 10 ng/mL cholera toxin (C8052; Sigma), 0.47 μg/mL hydrocortisone (H690; Sigma), 5 μg/mL insulin (10516; Sigma), and 5 ng/mL EGF (13247-051; Invitrogen). Cell exit from the spheroids is then monitored over time. Image analysis was performed by using Image J software and invasion of spheroids was calculated as a function of the longest invasive distance emanating from the spheroid. (Invasion=longest invasive distance−radius).

3D spheroid formation in Py2T and 4T1 cells: Spheroids are generated in drops of media that hang from the lid of a tissue culture dish for 72 hr. Basement membrane materials were mixed with the cell suspension at a 1:5 ratio and seeded onto a 24-well plate. After 30 min, culture media containing mAbs is then added to the wells. All cell cultures were incubated at 37° C. and 5% CO₂ incubator for 5 days, after which the tumor spheres were observed under an inverted microscope. The diameters of 30 randomly chosen tumor spheres were measured for each group.

Transwell migration and invasion assays: Cells were washed twice with 1× HBSS and harvested after trypsinization. For transwell migration assays, filters (8.0 μm pore size) and 24-well transwell chambers were used. Chambers were rinsed with culture medium without serum 1 hour before the assay. The cells were plated in triplicates in the upper wells at a density of 1× 105 per well in 0.1 mL of RPMI-0.1% BSA containing mAbs. Chemotaxis was induced using medium with 10% FBS on the bottom side of the chambers. Cells were allowed to migrate for a period of 48 hours at 37° C. and 5% CO₂ atmosphere, after which the experiment was stopped by wiping the cells from the upper side of the chamber with cotton swabs and fixed immediately with methanol for 15 minutes and then stained with 0.5% of Crystal violet for 15 minutes. A total of ten images were taken for quantification using an inverted microscope. The invasion assay was identical to the above migration assay except that filters were coated with 100 μl of the diluted Matrigel (BD Biosciences). The experiment was stopped after 48 hours as described in migration assay.

Activation assay and laminar flow adhesion assay: Cells were seeded in 6-well plates and cultured overnight. The cells were treated with 1 μg/ml control neutral mAb or activating mAb for 24 hr and cell adhesion activation was determined by the extent of morphological change to compact epithelial appearance. The laminar flow cell adhesion assay was conducted as described previously (Yap et al., Curr Biol 7:308-315, 1997; Chappuis-Flament et al., J Cell Biol 154:231-243, 2001). In brief, cells were trypsinized in the presence of 2 mM calcium and washed with lx HBSS. The cells were pretreated for 2 hr with 3 μg/ml neutral mAb, or activating mAb, and allowed to attach to glass capillary tubes coated with E-cadherin for 10 min, and washed away for 30 s at an indicated flow rate. The cells remaining after the wash were counted, and the adhesion percentage was calculated.

Statistics: Statistical analyses were performed using Graph Pad Prism software. Experimental values were expressed as the mean±SD based on three independent experiments, unless indicated otherwise. Statistically significant differences between two groups were determined using the nonparametric Mann-Whitney U test. P<0.05 was considered significantly different.

Results

Effects of rabbit mAb that recognize and activate mouse E-cadherin on the growth and metastasis of a spontaneous breast cancer mouse model

It was previously found that, in the 4T1 model of breast cancer, metastasis of an E-cadherin-expressing mammary cell line from the mammary gland to the lung depends on reduced E-cadherin adhesive function. MMTV-PyMT mice develop highly invasive mammary tumors that metastasize spontaneously to the lung and PyMT tumor cells highly maintained E-cadherin expression. How E-cadherin activating antibodies inhibit tumor metastasis was determined with the MMTV-PyMT, a murine syngeneic model of spontaneous breast cancer. This model closely mimics the progression of human breast cancer, advancing from hyperplasia through adenocarcinoma over a time course of approximately five months after birth. The experiment shown in the previous paper utilized activating mAb, 19A11 to human E-cadherin (Petrova et al. Molecular biology of the cell 27(21): 3233-3244, 2016). The 19A11 antibody could be advantageous for developing potential therapeutic agents in humans, but it does not recognize the mouse E-cadherin, resulting in complications for animal experiments. Therefore, rabbit mAbs were generated that recognize and activate mouse E-cadherin as well as control neutral mAbs, and the new activating mAbs were tested in colo205 cells lacking human E-cadherin.

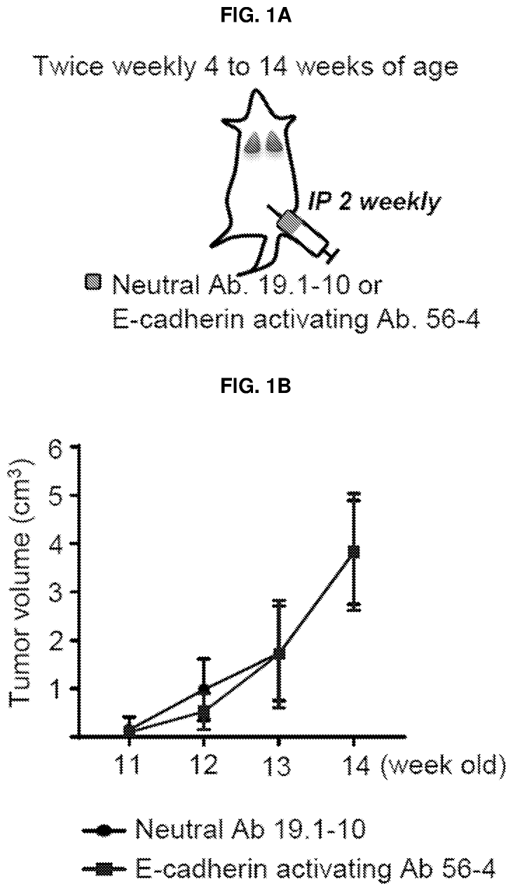

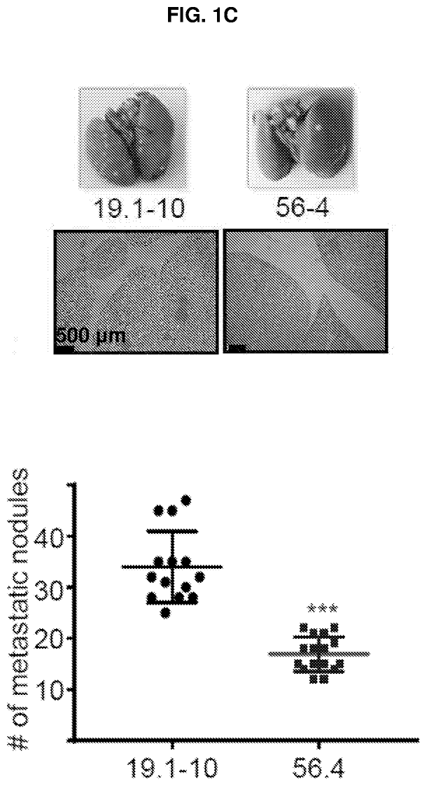

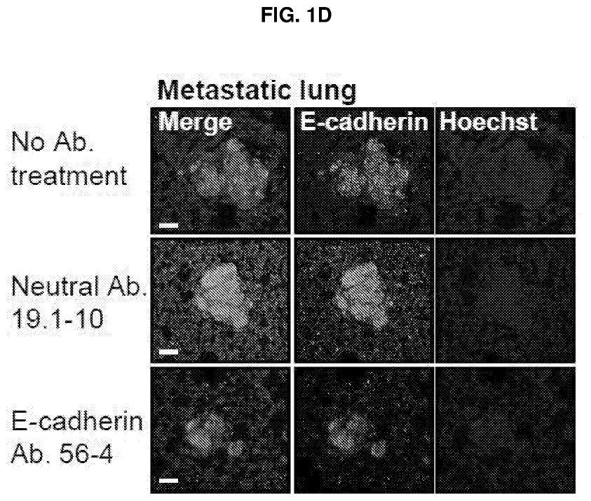

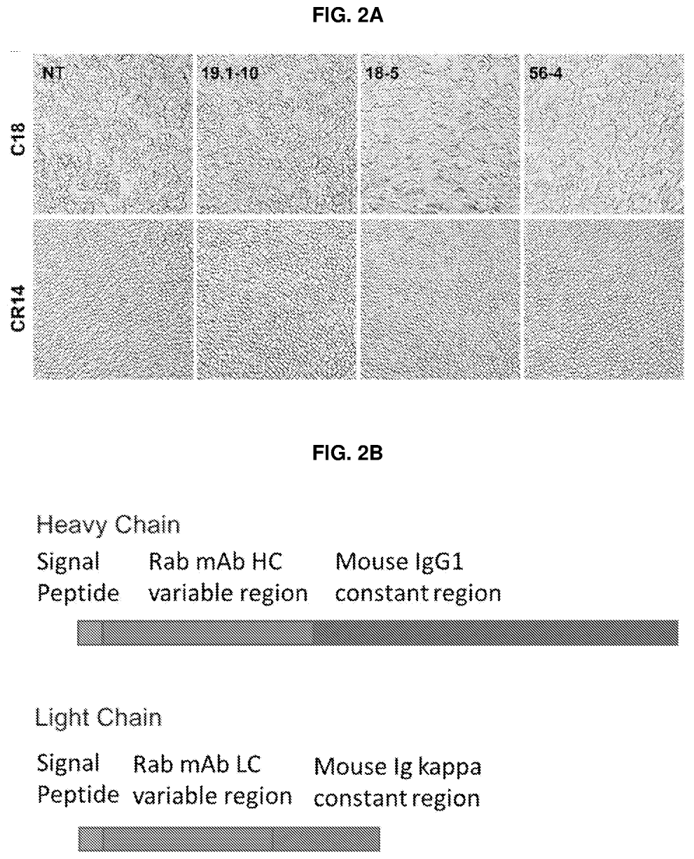

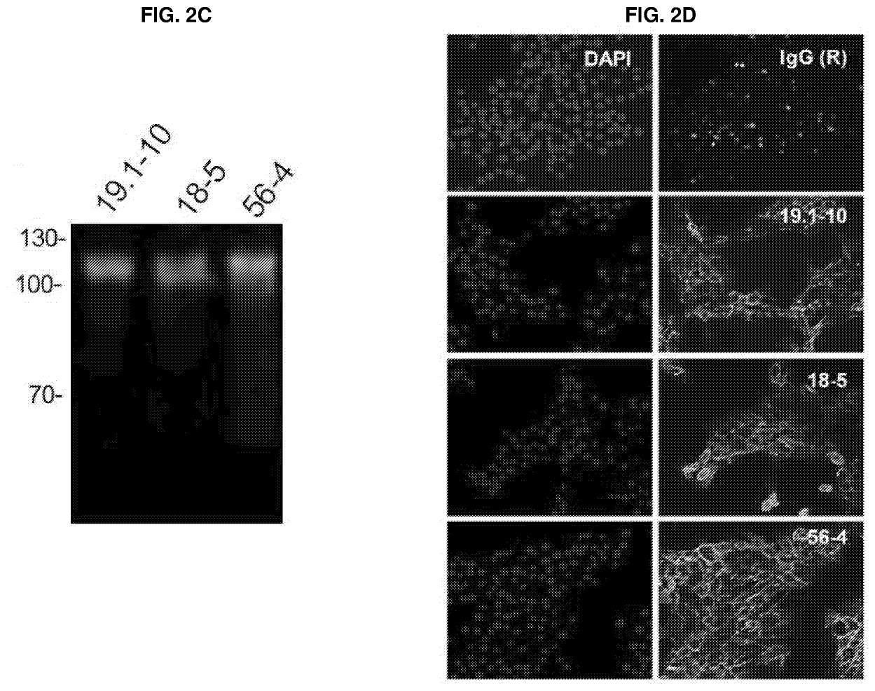

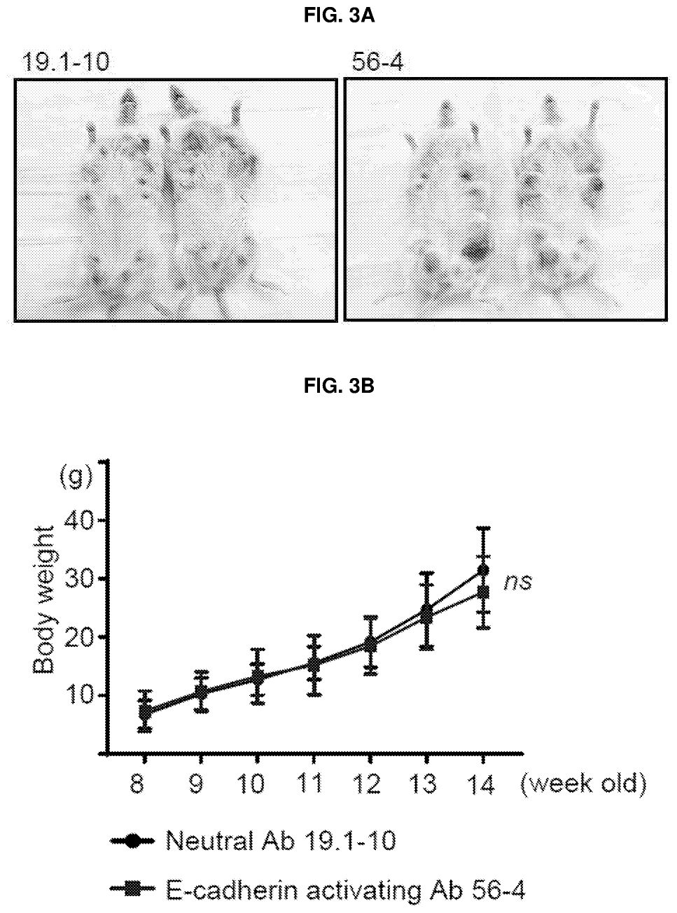

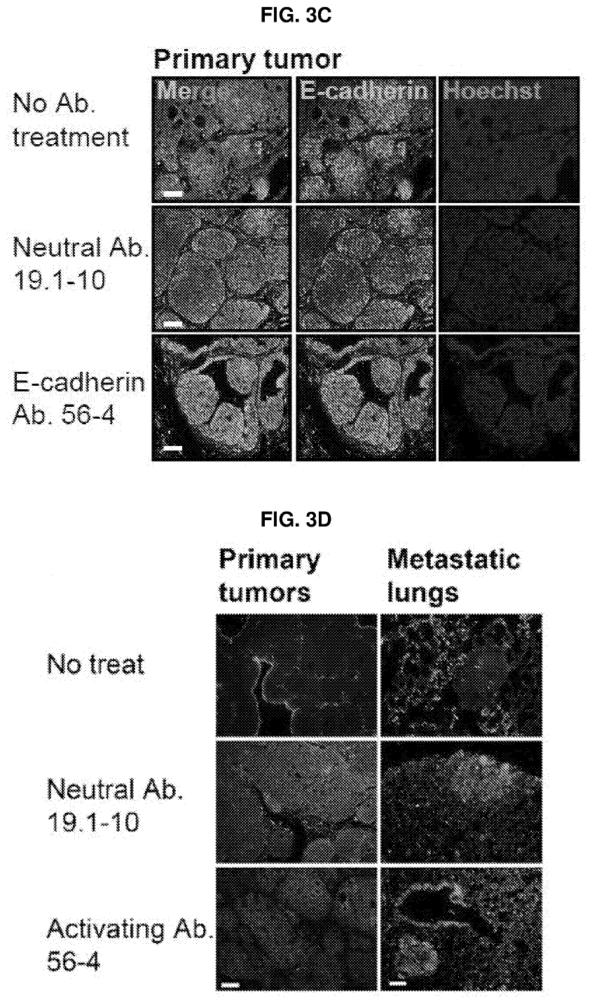

The new activating mAb, 56-4 effectively triggered the morphological change within 4 hr compared to neutral mAb, 19-1.10 (FIGS. 2A-2D). The female MMTV-PyMT or control mice were treated twice weekly with E-cadherin activating antibody 56-4 (5 mg/kg) or control neutral antibody 19.1-10 from 4 to 14 weeks of age (FIG. 1A). At 14 weeks of age, mice treated with 56-4 activating antibody displayed tumors in all 10 mammary glands, as did neutral antibody treated mice (FIG. 3A) and as with previous 4T1 mouse model, there was no difference in the growth in size of the primary tumor in the mammary gland for activating versus neutral mAb (FIG. 1B, FIG. 3B). Importantly, the number of metastatic lung nodules was largely reduced in the treatment of 56-4 activating antibody compared to 19.1-10 neutral antibody (FIG. 1C). Expression of E-cadherin was observed in both metastatic lungs (FIG. 1D) and primary tumors (FIG. 3C) treated with 56-4 activating antibody or 19.1-10 neutral antibody. Immunofluorescence staining with secondary Ab alone showed that the injected Abs were present in primary tumors and metastatic lungs (FIG. 3D). Although the total amount of E-cadherin-positive cells was reduced because of the decreased metastatic area by E-cadherin activating antibody (FIG. 1C), most of the cells that metastasized to the lung still expressed E-cadherin equally regardless of treatment with E-cadherin activating antibody (FIG. 1D). Therefore, progression and metastasis of E-cadherin positive tumors is controlled by the activity state of E-cadherin.

E-cadherin activation reduce the number of circulating tumor cells in mouse models of metastatic breast cancer

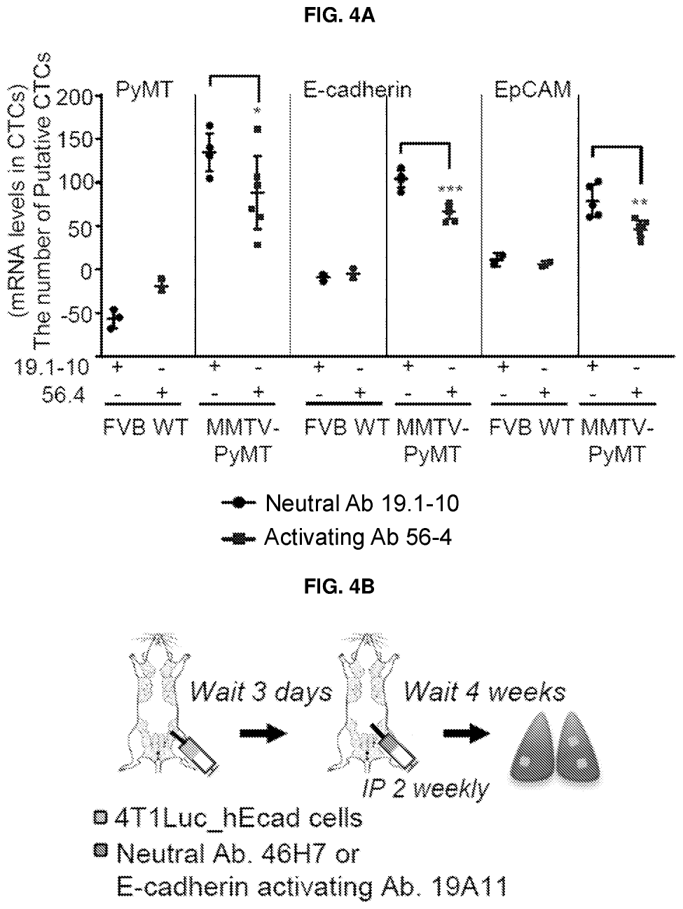

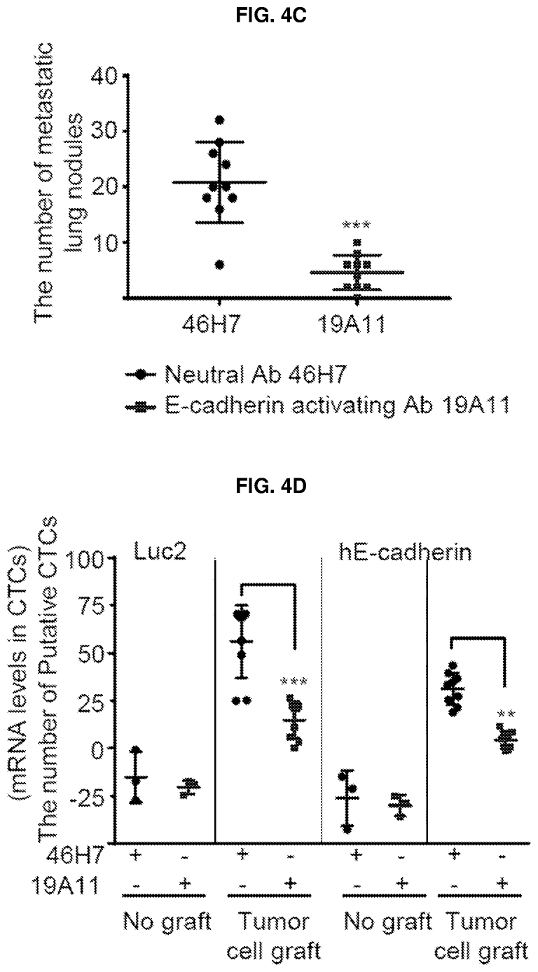

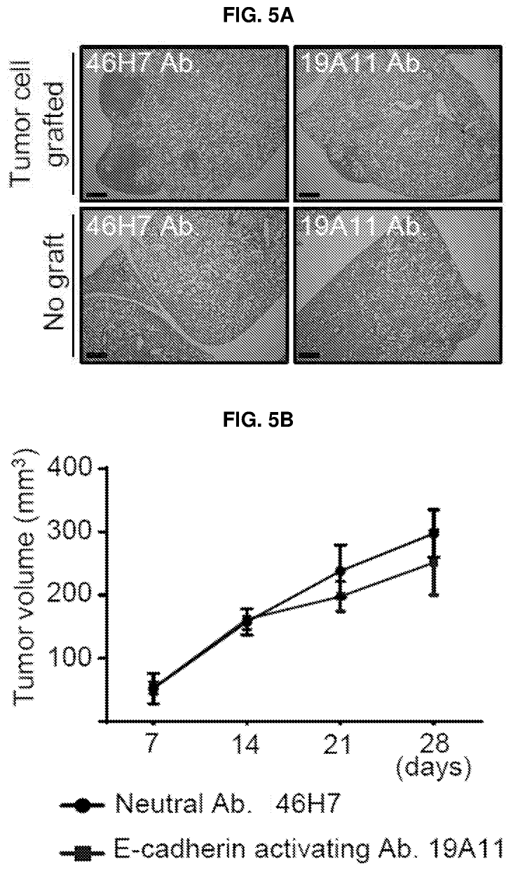

Next, a multi-step process was determined that can be affected by E-cadherin activation in the metastatic progression. Entry of tumor cells into the circulation, intravasation, is the critical first step in the development of distant metastases and detection of circulating tumor cells (CTCs) in blood samples can be a predictor of response to metastatic spread of carcinoma. To directly test whether disseminated tumor cells decrease by 56-4 activating antibody, the circulating tumor cells were first analyzed in tumor-bearing mice. After several centrifugations, RBC lysis, and several wash steps, CTCs amplified specific DNA fragments of PyMT and epithelial markers by quantitative real-time PCR (QPCR) as described in Materials and Methods. Also, since the most of isolated blood cells are monocytes, putative number of CTCs were calculated from equation for the expression of mRNA levels in cultured Py2T cells. Interestingly, treatment of 56-4 activating antibody significantly decreased total mRNA levels of PyMT, E-cadherin, and EpCAM expression compared to neutral antibody treatment in collected whole blood cells (FIG. 4A). To further characterize the inhibition of CTCs by E-cadherin activation, the 4T1 breast cancer cells transfected with both hE-cadherin and luciferase were utilized to trace CTCs in the experimental lung metastasis model. The in vivo alive mouse imaging (Pearl® Trilogy Imaging System, LI-COR, Inc.) was used to dynamically monitor the growth of primary tumor and metastasis to the lung by detecting Luciferase. 4T1 Luc2-hE cells were injected orthotopically into the mammary fat pad of balb/c mice. After 3 days, mice were treated twice weekly with 19A11 activating antibody (5 mg/kg) or control neutral antibody 46H7 for 4 weeks (FIG. 4B). As with previous results, the number of metastatic lung nodules was significantly reduced (FIG. 4C, FIG. 5A) even though there was no detectable difference in growth of primary tumor in the mammary gland for activating versus neutral mAb (FIG. 5B) (Petrova et al. Molecular biology of the cell 27(21): 3233-3244, 2016). CTCs amplified specific DNA fragments of luciferase and human E-cadherin genes by qPCR and confirmed that the analyzed CTCs were identical to the originally transplanted 4T1 Luc2-hE cells. Indeed, treatment of 19A11 activating antibody largely decreased the mRNA level of luc2 and hE-cadherin (FIG. 4D). Putative number of CTCs were calculated from equation for the expression of mRNA levels in cultured 4T1 Luc2-hE cells. Taken together, these data show that the inhibition of distant metastasis in the presence of E-cadherin activating mAb is associated with a decrease in the number of circulating tumor cells.

Activation of E-cadherin can repress metastatic colonization by extravasation into the lung parenchyma

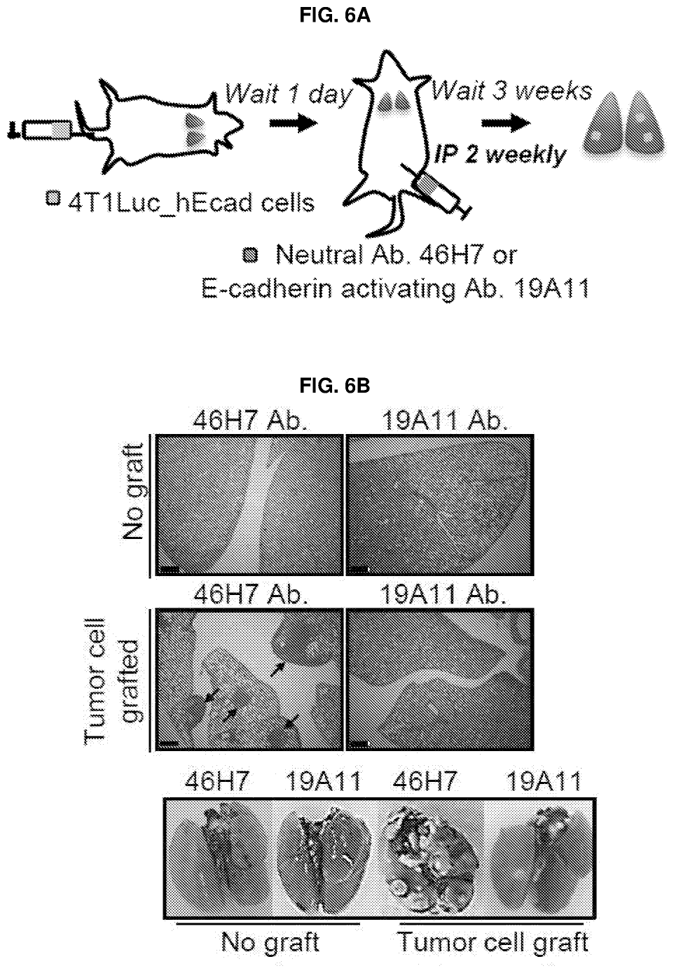

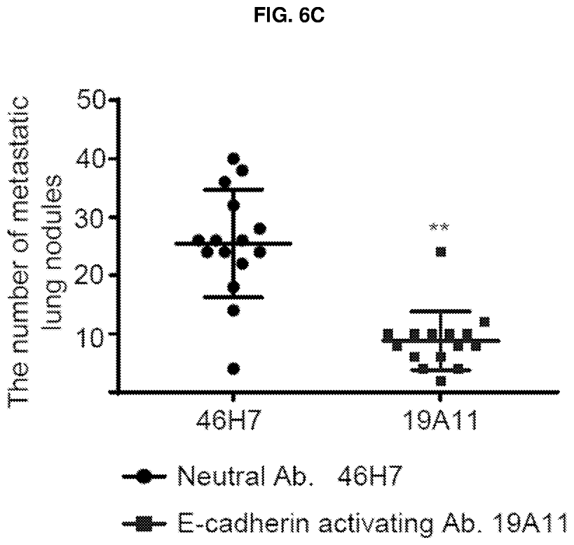

After intravasation the tumor cells can be detected as circulating tumor cells (CTCs) either in blood or lymphatic circulation. Circulating tumor cells in the blood must evade immune clearance (immune evasion), reach a capillary bed of a distal organ and invade through the endothelial cells of the blood vessel (extravasation) in order to successfully establish themselves. To investigate whether E-cadherin activation affects the extravasation from the vasculature, metastasis was induced by injecting tumor cells into the tail vein, a commonly used method to study later stages of metastasis. 4T1 Luc2-hE cells were injected intravenously and the mice were intraperitoneally treated with either 19A11 activating antibody or 46H7 neutral antibody for 3 weeks (FIG. 6A). In preliminary experiments, the in vivo alive mouse imaging indicated that small metastases began to form after 14 days and the visible metastatic lung nodules was also observed 14 days later. Interestingly, the 19A11 activating antibody was able to inhibit lung metastasis quickly and effectively even when the development of lung metastasis after 3 weeks of intravenous injection of 4T1-hE cells was significantly greater than four weeks after orthotopic injection into the mammary fat pad (FIGS. 6B and 6C). These results suggest that activation of E-cadherin may be an effective strategy in the process of CTC extravasation from the circulation.

Enhancement of E-cadherin activity inhibits the cell invasion in 3D culture of MMTV-PyMT mouse mammary tumor organoid and in vitro setting

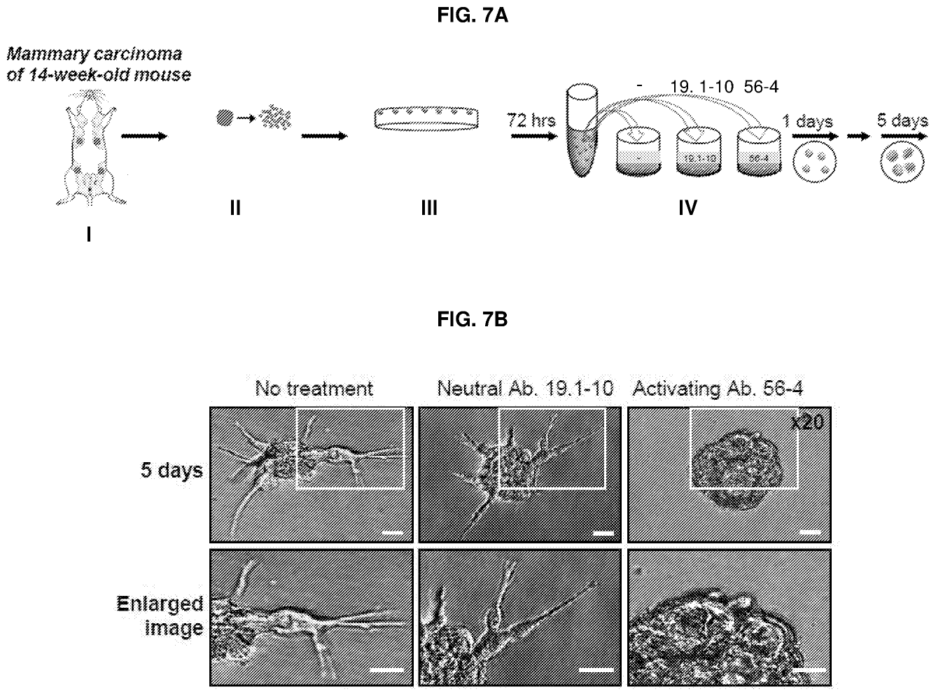

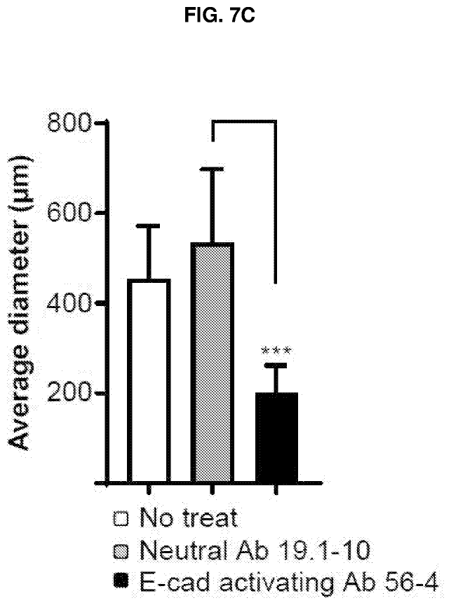

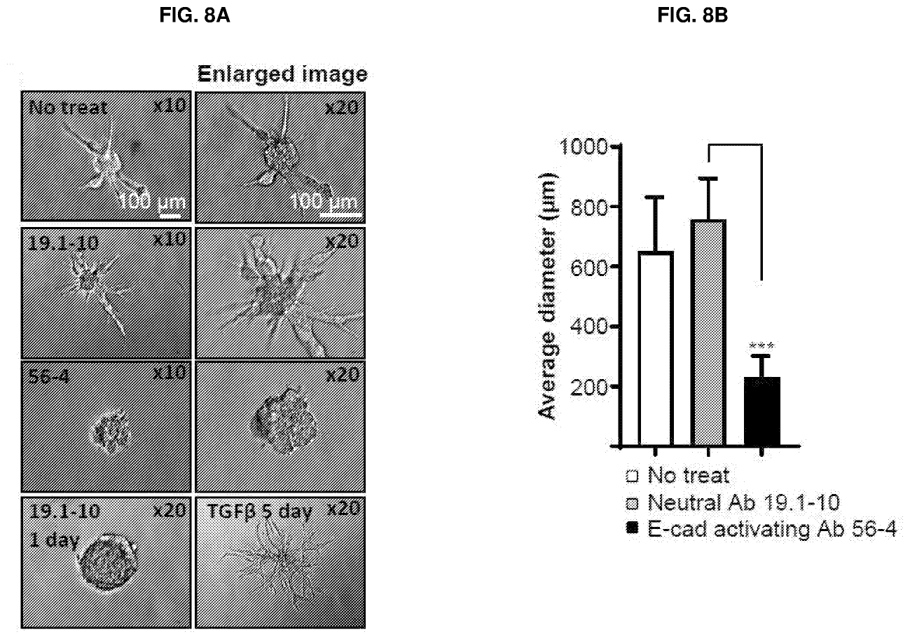

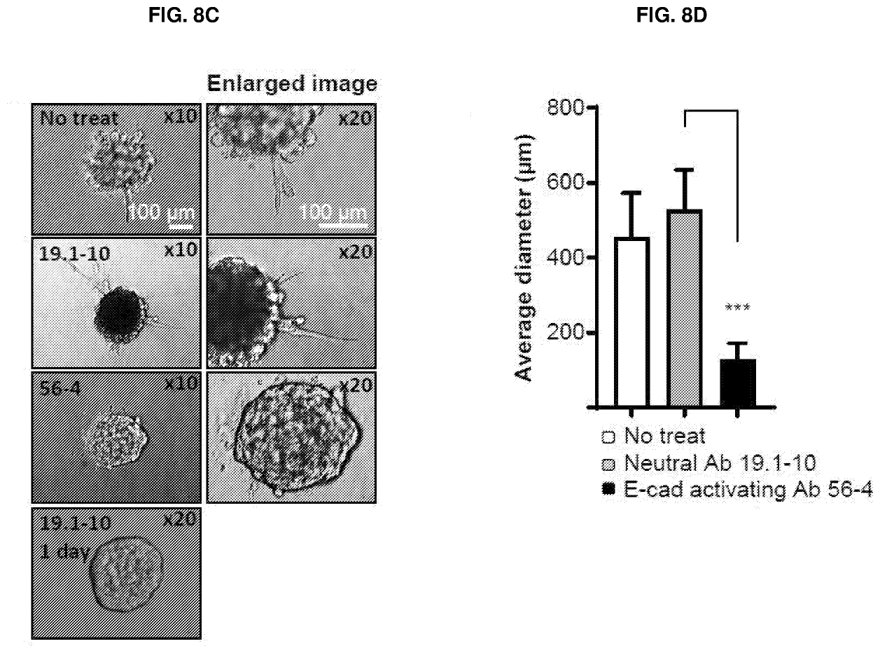

The loss of cell-cell adhesion capacity allows malignant tumor cells to dissociate from the primary tumor mass and changes in cell-matrix interaction cause the cells to invade the surrounding stroma. An epithelial cell in a mammary duct exists in a highly structured 3D environment and receives extensive inputs from cell-cell, cell-matrix, and soluble signals. Whether E-cadherin activation affects tumor cell invasion was next analyzed in an 3D in vitro model. Briefly, primary tumors (12-13 wk, 1.5- to 2.0 cm tumors) were isolated and used a combination of mechanical disruption and enzymatic digestion to generate “tumor organoids” and the organoid spheroids were embedded into 3D gel (FIG. 7A). Carcinoma fragments in 3D gels developed into budded structures with high efficiency after 5 days. The development of protrusions was detectable by transmitted light microscopy in the treatment of neutral antibody, but interestingly, activating antibody largely decreased the surrounding matrix and maintained the spheres (FIG. 7B). The quantification is obtained by calculation of invasion as a function of the longest invasive distance emanating from the cell spheroid body. In the presence of 56-4, the relative fold induction of spheroids was significantly reduced (FIG. 7C). To further test the inhibition of invasion in in vitro setting, the spheroids of Py2T and 4T1 cells were seeded into a 3D extracellular matrix. When cultured for 5 days in growth factor-reduced Matrigel, Py2T cells were highly protrusive and migratory in treatment of neutral antibody. In contrast, activating antibody 56-4 largely reduced the invasion (FIGS. 8A and 8B). Similarly, invasion of 4T1 cell spheroid was increased in the presence of neutral antibody, but was significantly inhibited when E-cadherin activating antibody 56-4 (FIGS. 8C and 8D). Thus, enhancement of E-cadherin functional activity may contribute to suppression of spread and invasion of carcinoma cells.

The crucial steps of the cancer metastatic process were regulated by E-cadherin activation

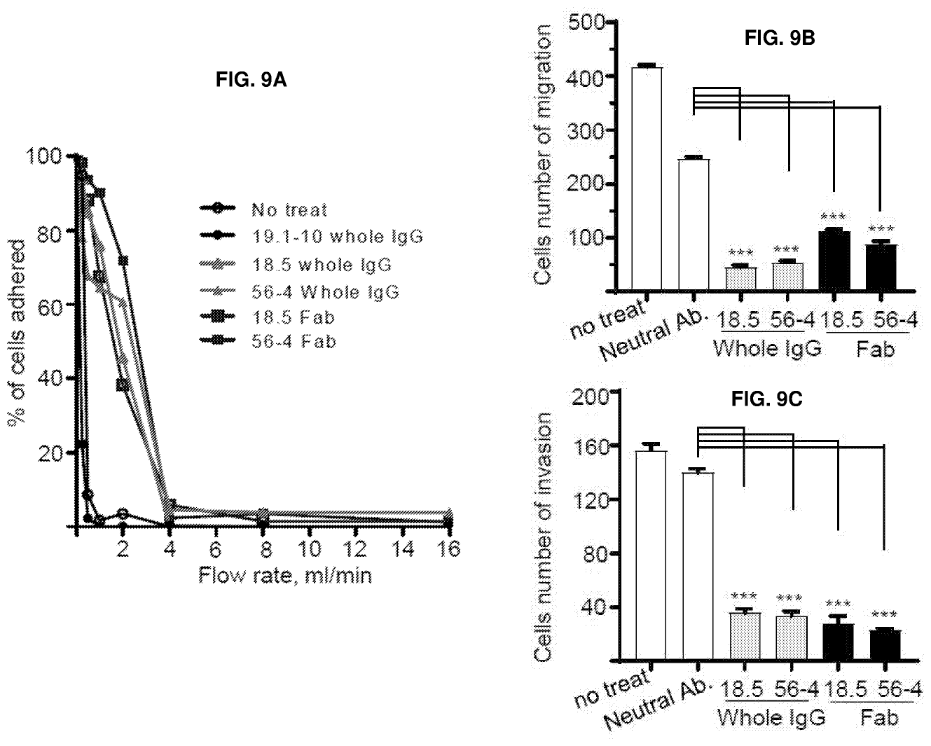

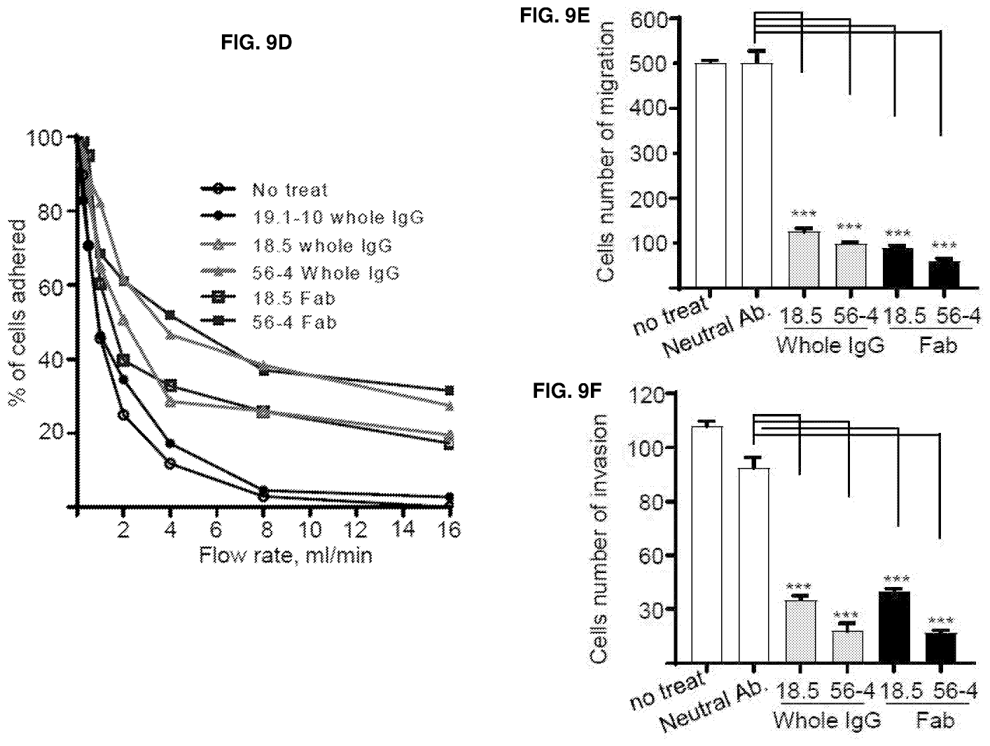

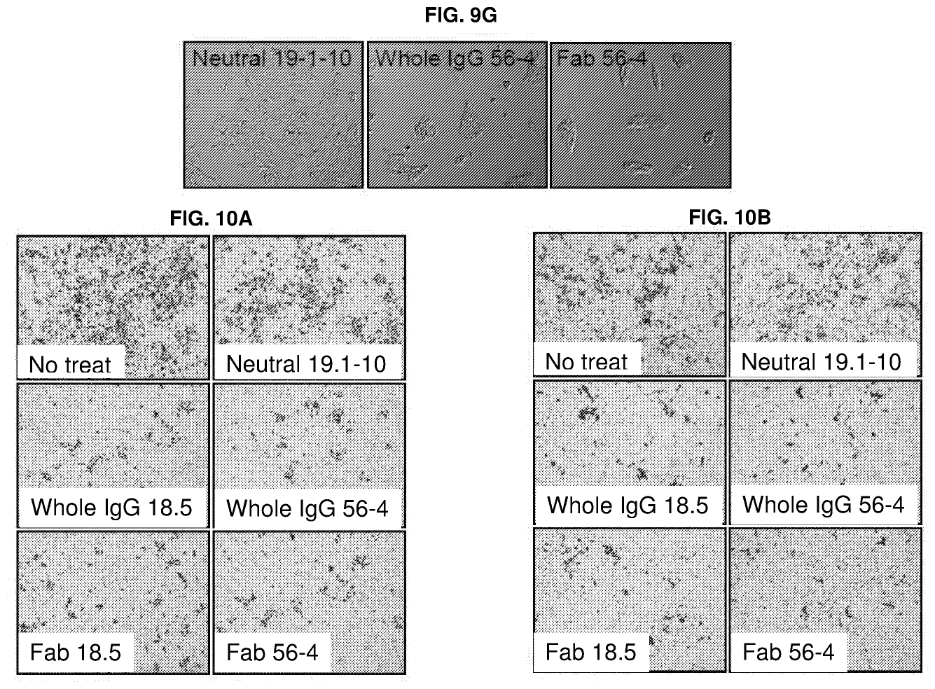

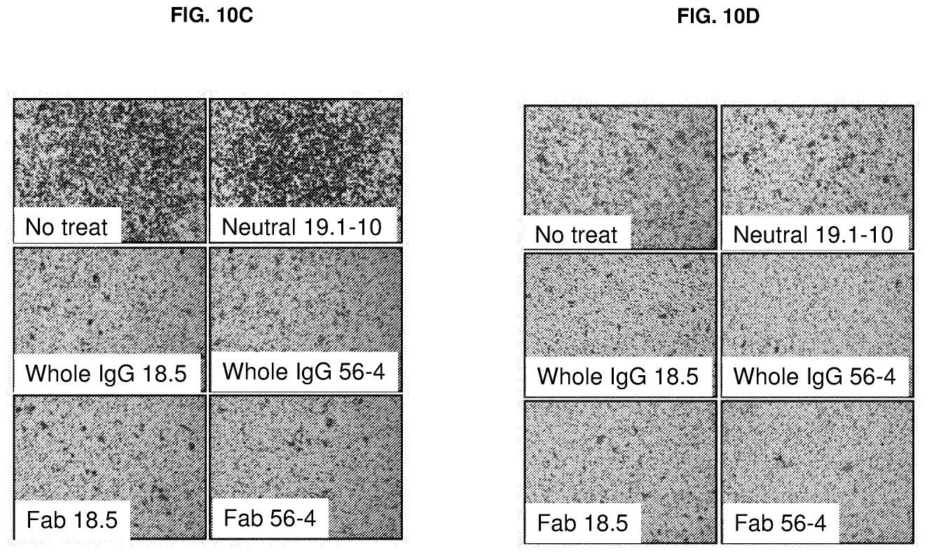

To further confirm the physiological mechanisms by which activating antibodies can inhibit tumor cells from escaping from primary cancers and causing metastasis through the bloodstream, adhesion, migration and invasiveness of the tumor cells were evaluated in vitro cell culture. In the presence of E-cadherin activating antibodies, whole IgG or Fab fragment of 18-5 and 56-4, cell attachment was largely increased in both cell line, Py2T (FIG. 9A) and 4T1 (FIG. 9D) compared to neutral antibody treatment. Unlike 4T1 cells, which retained a significant increase in cell adhesion by the activating antibody even though the flow rate increased, the cell adhesion rate of Py2T cells decreased rapidly as the flow rate increased but increased significantly at the initial flow rate compared with neutral antibody treatment (FIGS. 9A and 9D). To test the effect of E-cadherin activation on cell migration and invasion, Py2T and 4T1 cells were treated with 3 μg/ml of activating antibody or neutral antibody for 24 h and then migration and invasion assay were performed by transwell assay. Cell migration was significantly suppressed by treatment of whole IgG and Fab activating antibodies compared to neutral antibody in both cell line, Py2T (FIG. 9B, FIG. 10A) and 4T1 (FIG. 9E, FIG. 10C). Whole IgG and Fab activating antibodies also have inhibitory effect on the invasion ability of Py2T (FIG. 9C, FIG. 10B) or 4T1 (FIG. 9F, FIG. 10D). Additionally, the morphological changes were observed. Treatment of whole IgG and Fab activating antibodies in 4T1 cells resulted in condensed morphological changes (FIG. 9G). Overall, these data show that the four essential steps of the cancer metastatic process (detachment, migration, invasion and adhesion) can be regulated by E-cadherin activation.

Apoptosis in tumor cells expressing E-cadherin is regulated by the activity state of E-cadherin

Although E-cadherin activation increases cell adhesion and decreases cell migration and invasion, there may still be some questions about tumor cells that are detected in the blood (FIG. 4) and the metastatic organs (FIG. 1D). Metastatic cell dissemination requires that cells first detach from the primary tumor.

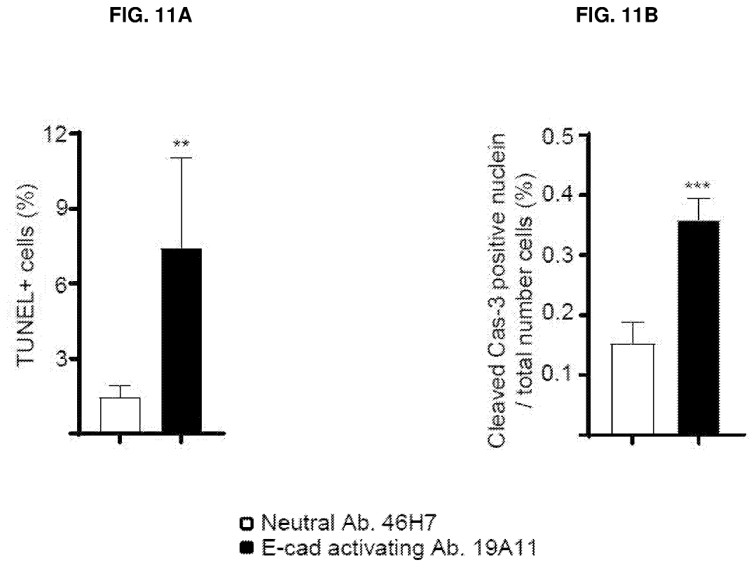

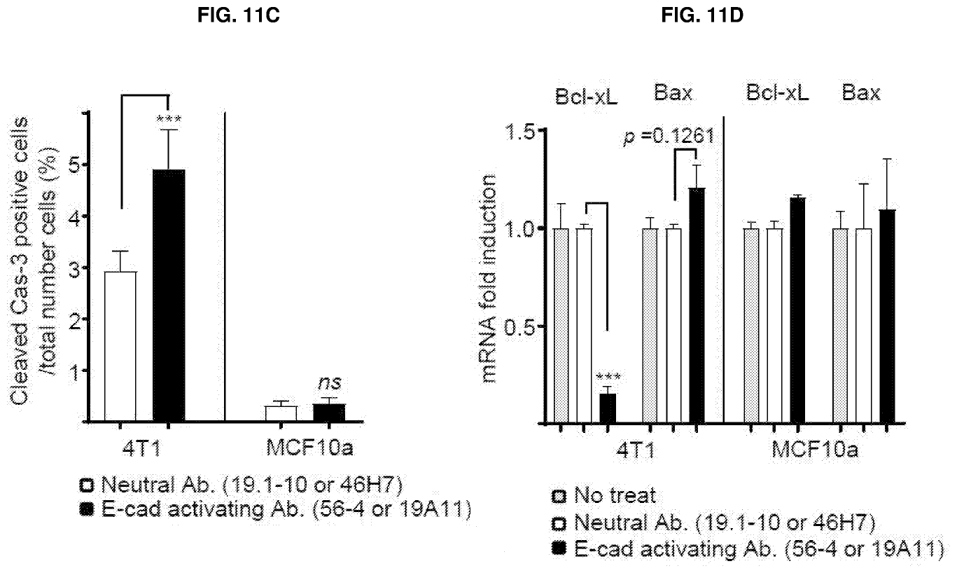

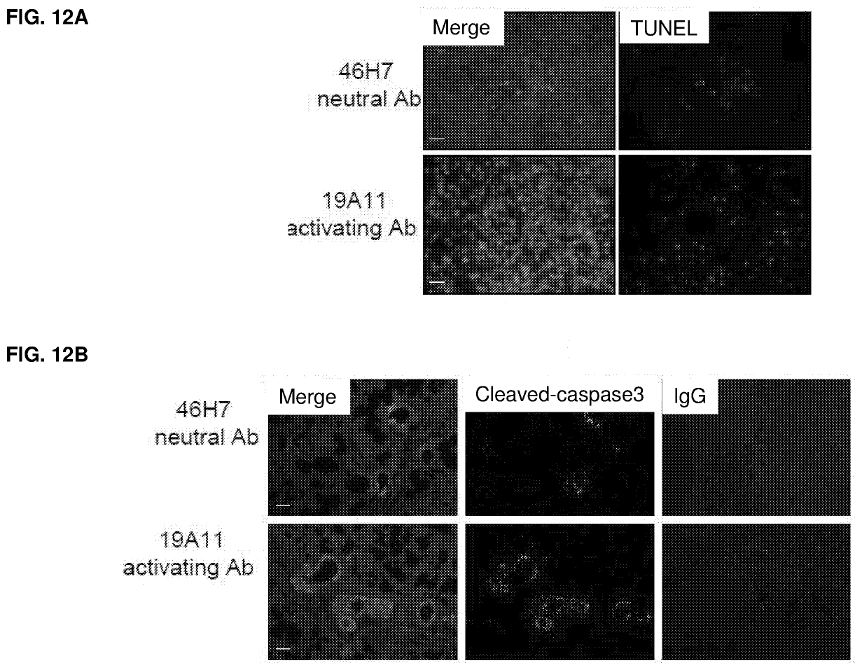

Under normal circumstances, epithelial and endothelial cells will undergo apoptosis (programmed cell death) when detached, a phenomenon referred to as anoikis (induction of apoptosis caused by detachment from the ECM). Moreover, expression of antiapoptotic molecules that confer resistance to anoikis has been shown to promote metastasis. To investigate whether this traditional concept is related to the results of inhibiting metastasis when treated with activating antibodies, the physiological phenomena of apoptosis were analyzed. For this purpose, a TUNEL assay was used to detect DNA fragmentation and analyzed. Surprisingly, more apoptotic cells increased in the metastatic lungs after treatment of 19A11 activating antibody (FIG. 12A). Quantitative analysis of TUNEL-positive cells demonstrated a significant increase of almost 5-fold in lungs treated with 19A11 activating antibody compared with neutral antibody treatment (FIG. 11A). Simultaneously, the apoptosis was confirmed with cleaved caspase-3, a specific apoptotic marker. An increased population of cleaved caspase-3 positive cells was observed in tumor lesions of the metastatic lungs treated with the 19A11 activating antibody (FIG. 11B, FIG. 12B). Next, 4T1 cells were cultured in vitro to further investigate the cell apoptosis by activating antibody. Treatment with activating antibody significantly increased the percentage of cleaved caspase-3-positive 4T1 cells (FIG. 1C) and compared with the neutral antibody, activating antibody largely decreased the mRNA level of antiapoptotic marker Bcl-xL. In contrast, the mRNA level of the proapoptotic marker Bax showed an increased pattern (FIG. 11D). Surprisingly, MCF10a, a normal breast cell, was not altered by antibody treatment (FIGS. 11C and 11D). These results suggest that E-cadherin activating antibody increases cancer cell-specific apoptosis.

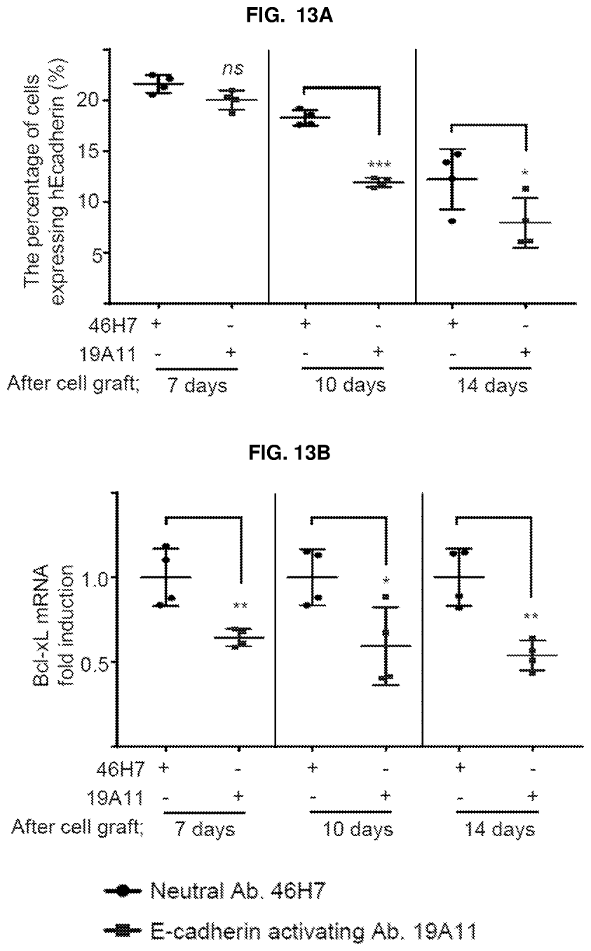

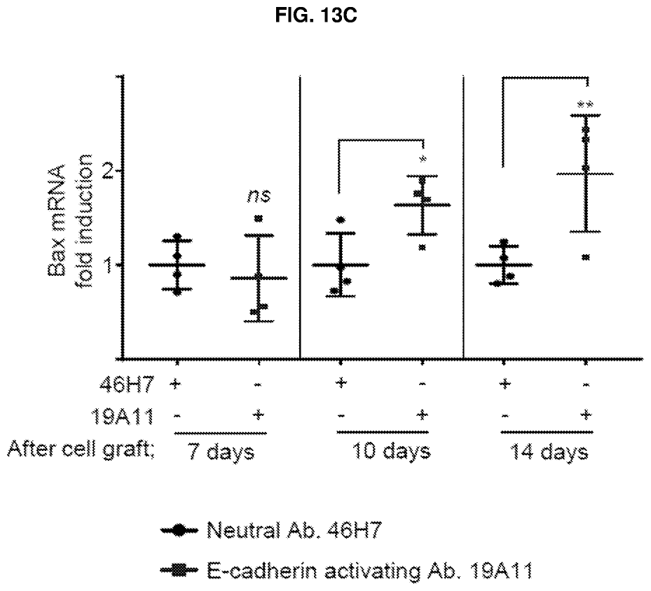

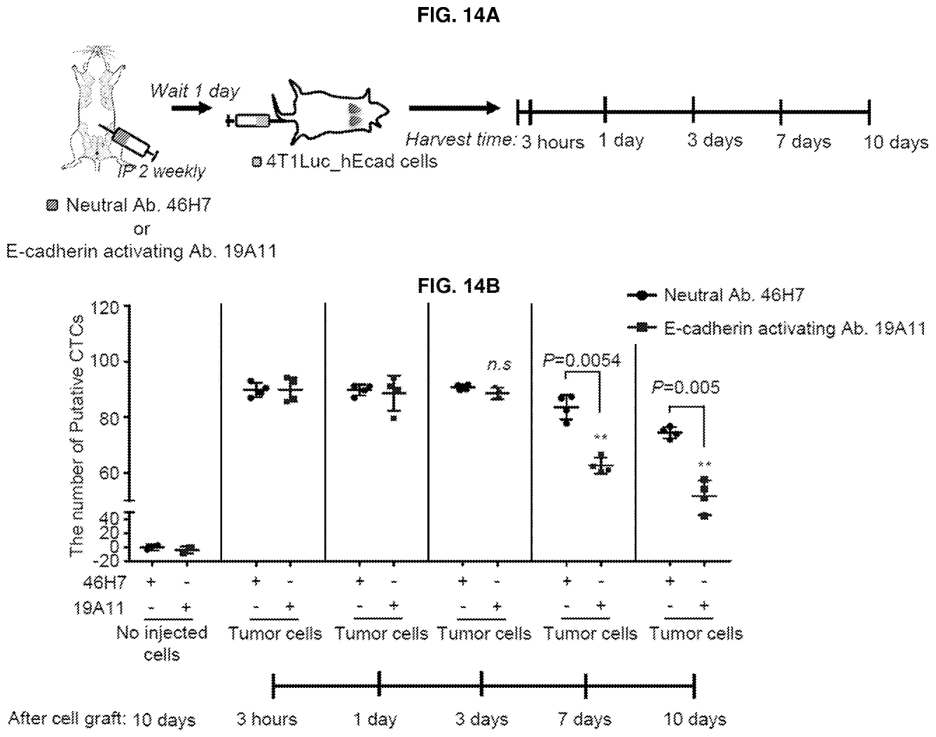

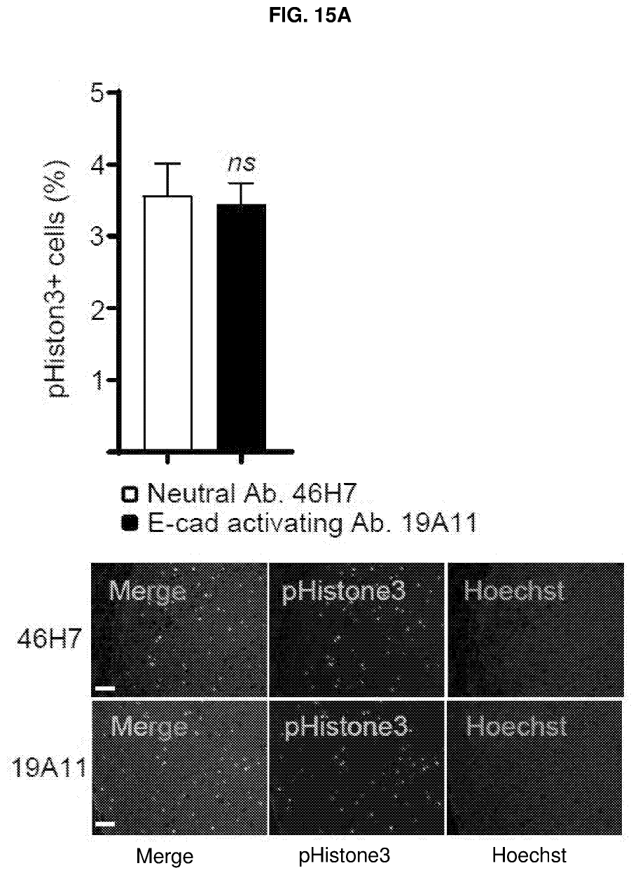

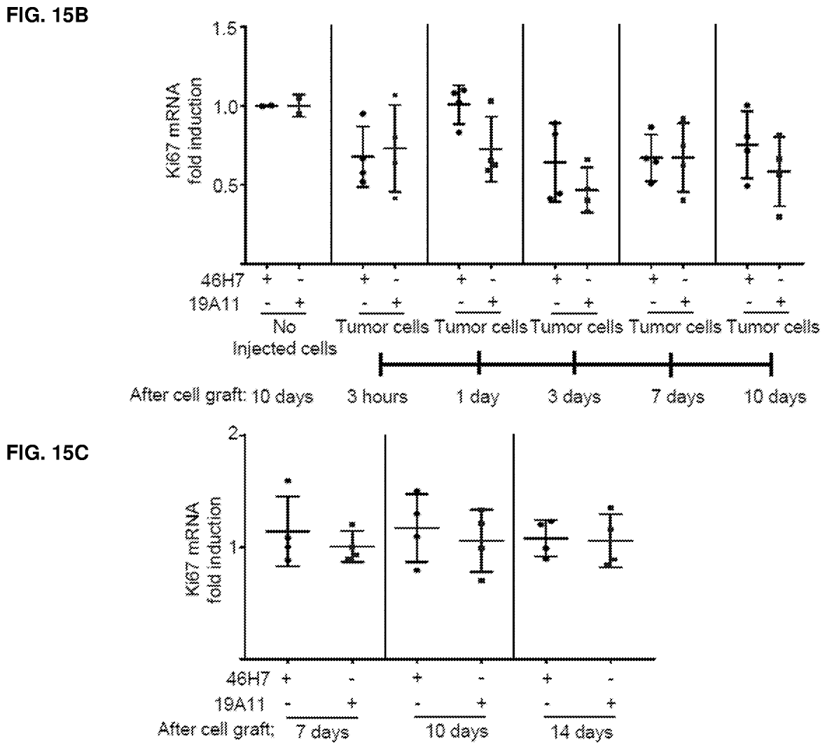

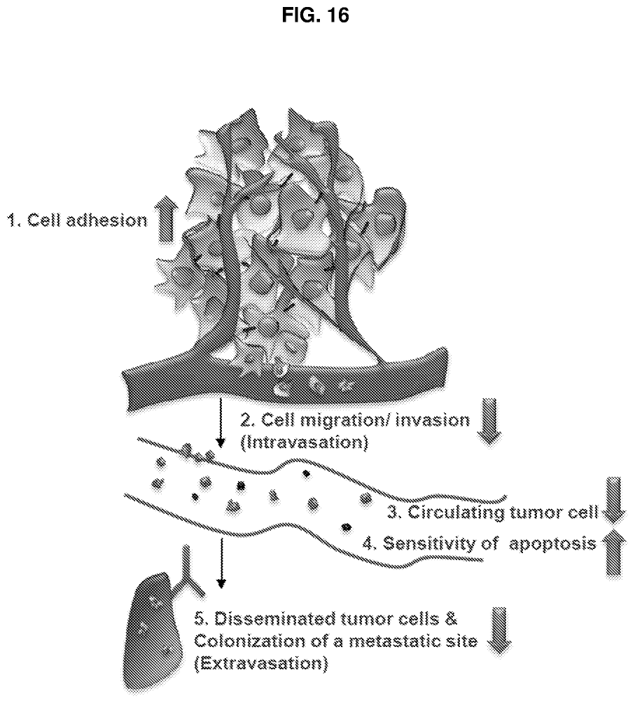

To determine whether the cells undergo apoptosis in the bloodstream before metastasis to the lung, the apoptotic circulating tumor cells were examined in the blood. First, the time-point at which the effects of cell apoptosis appeared in the preliminary test was confirmed. 19A11 activating antibody or 46H7 neutral antibody was injected the day before injection of 4T1-hE cells, and the circulating tumor cells were extracted and analyzed at the indicated time points (FIG. 14A). The number of circulating tumor cells was not altered by antibody treatment at the early time points, but a significant decrease was observed between 7 and 10 days by treatment of 19A11 activating antibody (FIG. 14B). Surprisingly, the injected cells were 30 thousand cells, but the number rapidly decreased after 3 hours and only about 100 CTCs were detected and the number was further reduced over time (FIG. 14B). This might relate to a balance between the number of cancer cells entering the circulation or lymph nodes and their clean-up by the immune cells. Putative number of CTCs were calculated from equation for the expression of mRNA levels in cultured 4T1 Luc2-hE cells. Based on these results, mRNA level of apoptotic markers was measured at 7, 10 and 14 days after cell injection. According to the previous paper, myeloid-derived suppressor cells (MDSC) modulates Bax and Bcl-xL expression to regulate the Fas mediated apoptosis pathway (Hu et al. J. Biol. Chem. 288: 19103-19115, 2013). Therefore, 4T1 cells expressing hE-cadherin were sorted by FACS analysis. Before sorting the cells, the proportion the cells expressing hE-cadherin was first analyzed by flow cytometry as described in the materials and methods. 19A11 largely decreased the percentage of cells expressing hE-cadherin, indicating that the E-cadherin activating antibody inhibited circulating tumor cells as shown in the results of the orthotopic injection of FIG. 4 (FIG. 13A). Next, mRNA levels of apoptotic markers were measured by qRT-PCR in the sorted cells. In the presence of E-cadherin activating antibody 19A11, the mRNA level of Bax was largely increased (FIG. 13B) but Bcl-xL mRNA was significantly reduced by treatment of 19A11 (FIG. 13C). It was previously found that there was no quantitative difference in the cell number expressing the proliferation marker Ki67, consistent with the lack of effect on primary tumor size (Petrova et al. Mol. Biol. Cell 27: 3233-3244, 2016). Consistent with the results, proliferation markers pH3 positive cells did not alter by activating antibody in the metastatic lungs as well as primary tumors (FIG. 15A). In addition, when mRNA level of proliferation marker Ki67 was measured in a time-dependent manner by qRT-PCR, the treatment of 19A11 did not affect overall cell proliferation in both unclassified circulating tumor cells (FIG. 15B) or sorted circulating tumor cells (FIG. 15C). Therefore, E-cadherin activating antibodies can inhibit metastasis by inducing cancer cell-specific apoptosis in the bloodstream prior to metastasis to distant organs without affecting cell proliferation. Taken together, these results demonstrate that E-cadherin activation can inhibit the overall process of metastatic cascade includes local invasion, intravasation, dissemination, extravasation, and colonization at the target organ (see FIG. 16).

### Example 3: Enhancing Epithelial Barrier Function

This example describes new approaches to target and treat diseases and conditions influenced by defective or disrupted epithelial barrier function (such as inflammatory bowel disease (IBD) and conditions impacting respiratory system permeability, such as acute respiratory distress syndrome (ARDS), asthma, and respiratory infections). These approaches involve preventing or treating disease by enhancing barrier function, through activation of E-cadherin.

Described herein are ways to activate cell-cell adhesion and cell-cell junction formation, using activating monoclonal antibodies to E-cadherin (such as mAbs 19A11, 66E8, 56-4, 18-5, and humanized versions and functional fragment thereof). These mAbs are used to target intestinal epithelium rather than immune cell compartment.

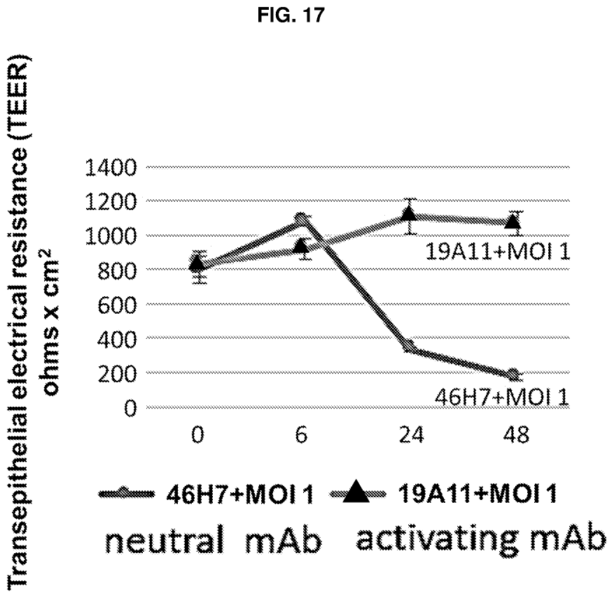

Activating mAbs to human E-cadherin inhibit the loss of human airway cell monolayer permeability caused by respiratory syncytial virus (RSV) infection. Human bronchial epithelial cell line 16HBE14O- (1.5×10⁵ cells from passage number between 12-16) were grown in transwell chambers (Costar catalogue #3470: 6.5 mm insert with 0.4 μm pore size) at liquid:liquid interface for one week to form a confluent monolayer. Cells were then either treated with activating monoclonal E-cad 19A11 Fab or with neutral monoclonal E-cad 46H7 Fab at 3 μg/ml concentration for 4 hours. 19A11 Fab treated or 46H7 Fab treated cells were then either left uninfected or infected with RSVL19 with MOI of 1 for 6, 24, and 48 hours. Trans epithelial electrical resistance (TEER) in each Fab-treated 16HBE14O- monolayer was measured before and after of RSVL19 infection (MOI 1) at indicated time points (FIG. 17) using STX chopstick electrode and EVOM (epithelial voltmeter instrument). TEER values for blank filter was subtracted from each individual data point to determine the true tissue resistance for the monolayer. Unit area resistance was measured in triplicate and calculated for each condition and average values (ohms×cm²) were plotted in Y axis against time (hrs) at X axis. Error bars represent the average±standard deviations from three independent transwells.

High TEER means that epithelial junctions in cultured airway cells are sealed. Infection with RSV causes loss of TEER. As shown in FIG. 17, the Fab of mAb 19A11 (a representative activating antibody specific for human E-cadherin) inhibits loss of human airway cell monolayer permeability (that is, prevents loss of TEER) that is otherwise caused by RSV infection. Neutral mAb 46H7 is control antibody to E-cadherin, but that does not activate it. Therefore, activating mAb maintains junctional seal during RSV infection.

Gastrointestinal (GI) Barrier in Etiology of IBD: The intestinal epithelium (when healthy) separates microbes, immunogens, and toxins in intestine contents from the immune/inflammatory compartment. Increased intestinal permeability is associated with IBD—it precedes overt disease (demonstrated in both human and mice) and exhibits detectable familial association in humans. Modulation of GI epithelial barrier function is controlled by the activity state of E-cadherin at the cell surface and p120-catenin phosphorylation. IBD-associated and/or inflammatory processes downregulate E-cadherin adhesive activity.

Since GI barrier permeability had been strongly implicated in the etiology of IBD, it is proposed that the E-cadherin activating mAbs described herein, as well as p120-catenin mutations, can be used to enhance GI barrier function and slow the progression of IBD. Activating E-cadherin (for instance, using the activating mAbs described herein) will enhance GI barrier function and thereby reduce inflammatory responses and the progression of IBD. Also contemplated is use of activating mutations in p120-catenin (a cadherin associated protein) to influence intestinal epithelium permeability.

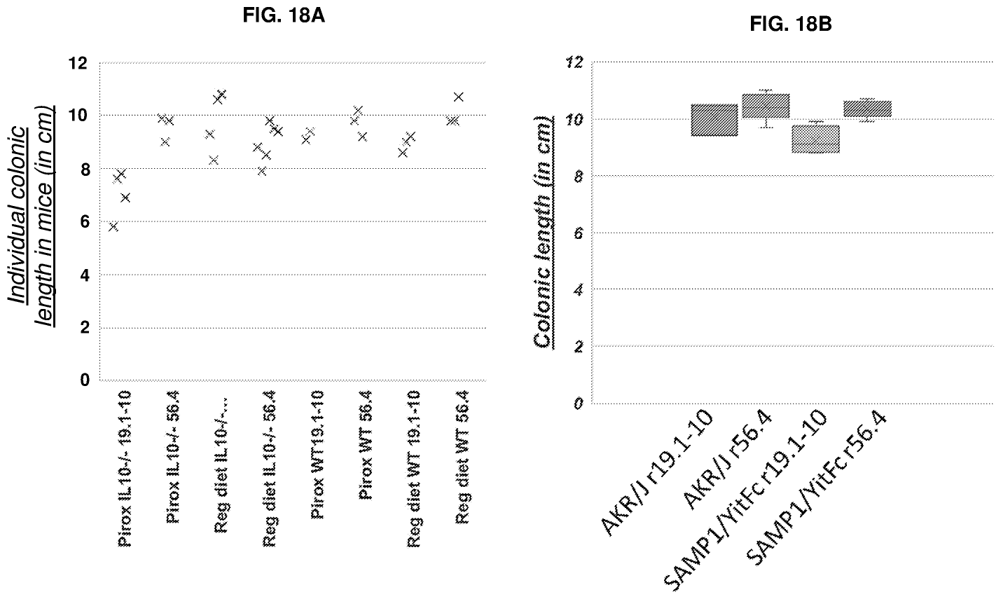

Effect of E-Cadherin antibody in colonic length in mice. FIG. 18A. Age matched male and female (5 weeks old) IL10KO mice strain B6.129P2-1110tm1Cgn/J and corresponding control mice strain C57BL/6J animals were either fed standard lab based rodent diet or fed with NSAID (non-steroidal anti-inflammatory) group of drug Piroxicam (at a dose of 200 ppm=200 mg/kg) pelleted lab based rodent diet for 14 days. All four treatment groups were intraperitoneally injected either with activating monoclonal E-Cad antibody r56.4 or with control neutral antibody r19.1-10 for two weeks at a dose of 5 mg/kg twice weekly. At 7 weeks of age animals were euthanized followed by colonic length measurement in each individual mouse. The values for colon length in cm are plotted in Y axis for indicated experimental cohorts. Total number of mice participant in each cohort ranged between 4-5. FIG. 18B. Age matched male (6 weeks old) spontaneous ileitis mice strain SAMP1/YitFc and corresponding control mice strain AKR/J were intraperitoneally injected either with activating monoclonal E-Cad antibody r56.4 or with control neutral antibody r19.1-10 for 4 weeks at a dose of 5 mg/kg twice weekly. At 10 weeks of age animals were euthanized followed by colonic length measurement in each individual mouse. The values for colon length in cm are plotted in Y axis for indicated experimental cohorts. Total number of mice participant in each cohort ranged between 4-5.

Activating E-cadherin-specific mAb, compared to control neutral mAb, counteracted the decrease in colonic length due to intestinal inflammation in both the IL10−/− model and the SAMP model. A decrease in colonic length is associated with intestinal inflammation; it is believed this may be due to loss of crypts or reduced capacity for crypt regeneration (though the results provided herein are in no way limited by this proposed mechanism). These results suggest that the activating mAb has reduced inflammation in these models.

### Example 4: E-Cadherin Activation Enhances GI Barrier Function

Dysfunctions in the integrity of the gastrointestinal epithelial barrier are thought to play a role in the onset and progression of inflammatory bowel disease (IBD). E-cadherin is an adhesion molecule required for controlling the epithelial barrier. This example describes approaches to treat and/or ameliorate symptoms associated with inflammatory bowel disease (IBD) by enhancing GI barrier function through activation of E-cadherin. The studies investigate whether mAbs that activate E-cadherin can slow the progression of IBD in two mouse models of the disease.

Adoptive T cell Transfer Model of Colitis.

This model is an acute induced model involving adoptive transfer of an inflammatory subset of T-cells into mice. Naïve T cells (CD4+CD45RB^{high}) are transferred from healthy wild-type mice into syngeneic recipients that lack T and B cells. The introduction of these T cells into the immunodeficient mice induces a pancolitis and small bowel inflammation at 5-8 weeks following T cell transfer (Powrie, Immunity 3:171-174, 1995; Powrie et al., Immunity 1: 553-562, 1994). Distal colon obtained from mice in this model reveals transmural inflammation, epithelial cell hyperplasia, polymorphonuclear leukocyte (PMN) and mononuclear leukocyte infiltration, crypt abscesses, and epithelial cell erosions by histopathological inspection. Mice reconstituted with inflammatory T cells exhibit varying degrees of weight loss, diarrhea, and loose stools, depending on the strain of the donor and recipient. This model allows observation of the very earliest immunological events associated with the induction of gut inflammation as well as the perpetuation of disease. The T cell transfer model is responsive to a variety of different immunological and antibiotic treatment protocols. Reports have shown that transfer of T cells into recombinase activating gene-1-deficient (RAG^{-/-}) mice induces both colitis and small bowel inflammation, rendering this model similar to Crohn's disease (Laroux et al., Int Immunol 16: 77-89, 2004; Ostanin et al., Am J Physiol Gastrointest Liver Physiol 292: G1706-G1714, 2007). The adoptive T cell transfer model can be used to study the role of regulatory T cells in suppressing or limiting the onset and/or perpetuation of intestinal and colonic inflammation.

Acute colitis was induced using a standard method by i.v. injection of a sorted subset of reactive T-cells, CD45Rb-high. CD45Rb-low T-cells, which do not cause colitis, were injected as a control. Monoclonal antibodies (mAbs) that activate E-cadherin and enhance epithelial barrier function were generated as described herein. The mAbs used in this experiment were specific to mouse E-cadherin, in contrast to the airway cell experiment (Example 3) and crystallography experiment (Example 5), which used mAbs specific to human E-cadherin. CD45Rb-high injected mice were subsequently treated by twice weekly IP injection of 5 mg/kg either E-cadherin activating mAb 56-4 or a control neutral mAb 19.1.10. Animals were monitored for weight loss. Colons were dissected from euthanized mice and measured. Histology of colon tissue was performed blind by HistoTox Labs (Boulder, Colo.). Standardized IBD scoring included generation of Sum Colitis Scores which includes the following parameters: Mean Edema Extent; Mean Histopathology Scores, which includes scores for mucosal thickening (hyperplasia), degree of inflammation, gland damage, and erosion extent; and Mean Neutrophil Score, which measures neutrophil invasion (score increases in inflammation).

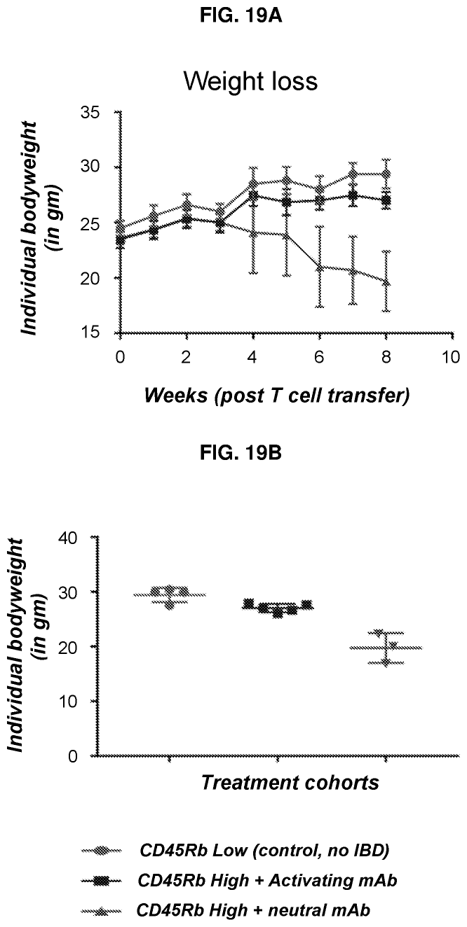

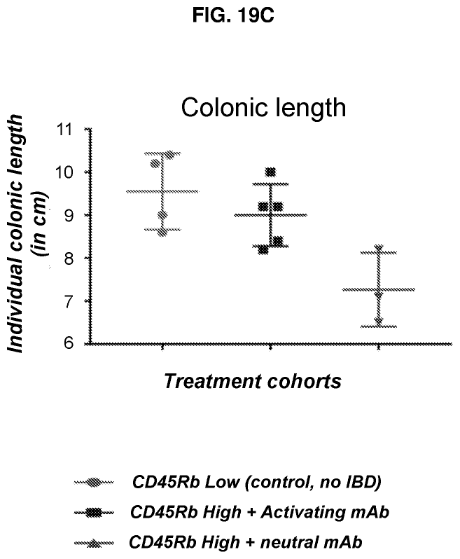

Activating mAbs Reduce Overt Symptoms. Compared to a neutral control mAb, E-cadherin activating mAb reduced weight loss and colon shortening caused by IBD induction (FIGS. 19A-19C). CD45Rb-high animals began to lose weight due to the onset of colitis, and this effect was reversed by activating mAb but not neutral mAb (FIG. 19A). FIG. 19B shows the endpoints of weight loss when the experiment was terminated. Colonic length is known to shorten in colitis. E-cadherin activating mAb was able to reduce colon shortening compared to the control neutral mAb (FIG. 19C).

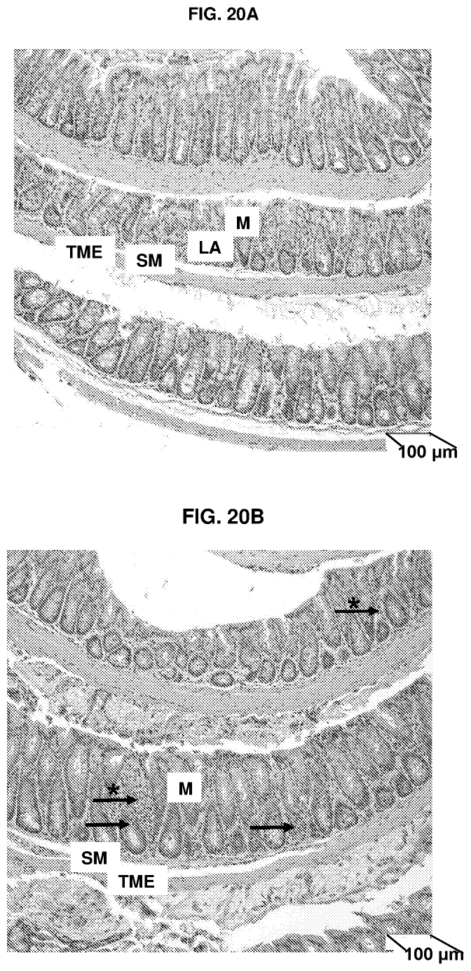

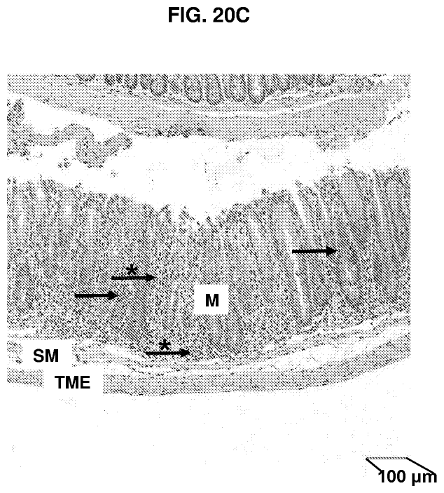

E-Cadherin Activating mAb Reduced Signs of Inflammation in Colitis. Pathohistological examination indicated that E-cadherin activating mAb significantly reduced signs of inflammation (FIGS. 20A-20C). FIG. 20A shows histology of a minimally affected colon from a mouse injected with CD45Rb-low T cells and treated with activating mAb 56.4. Mucosal glands (M), submucosa (SM), tunica muscularis externa (TME), and a lymphoid aggregate (LA) are indicated. FIG. 20B shows histology of a colon from a mouse injected with CD45Rb-high T cells and treated with activating mAb 56.4. The mucosal glands (M) are minimally to mildly hyperplastic. Multifocally, inflammatory cells (neutrophils, macrophages, lymphocytes; arrows with asterisks) infiltrate the lamina propria of the mucosa and separate glands. Occasionally, regions of inflammation are associated with crypt infiltration and damage (arrows without asterisks). The submucosa (SM) and tunica muscularis externa (TME) are indicated. FIG. 20C shows histology of a colon from a mouse injected with CD45Rb-high T cells and treated with neutral mAb 19.1-10. The mucosal glands (M) are mildly to moderately hyperplastic. Inflammatory cells (macrophages, lymphocytes, and occasional neutrophils; arrows with asterisks) infiltrate the lamina propria of the mucosa and separate glands. Regions of inflammation are associated with crypt infiltration and damage (arrows without asterisks) resulting in some gland loss. The submucosa (SM) is minimally expanded by edema. The tunica muscularis externa (TME) is indicated.

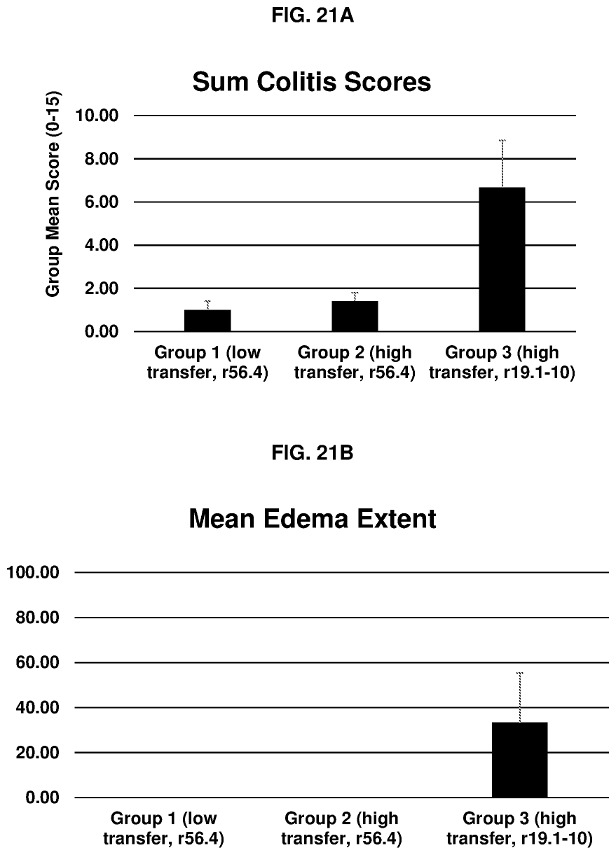

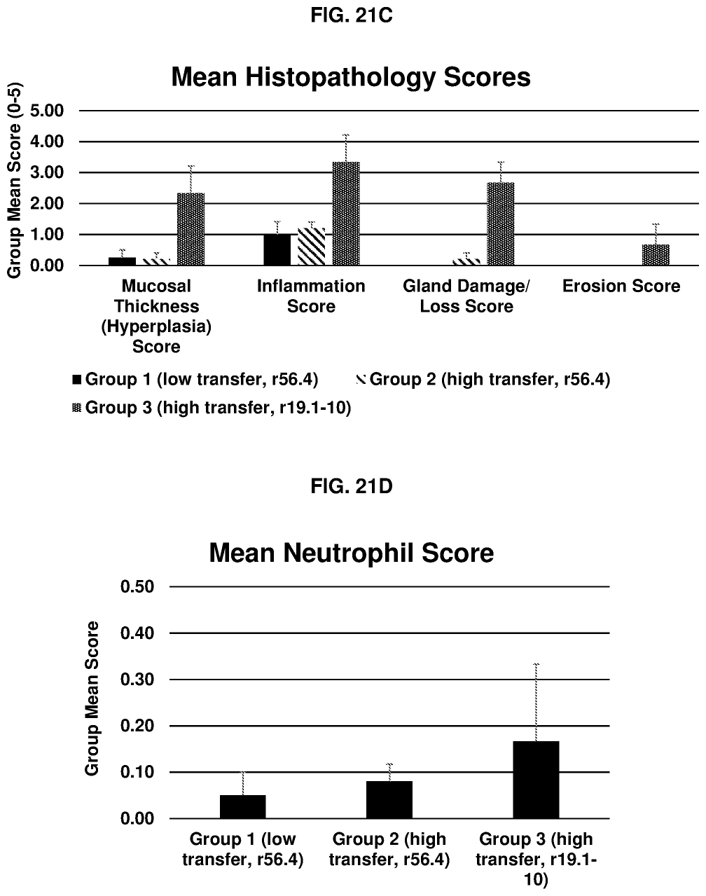

E-Cadherin Activating mAb Reduced Pathology of Colitis. Standard IBD histological analysis (carried out under contract with HistoTox, Boulder, Colo.) shows that E-cadherin activating mAb significantly suppressed IBD (FIGS. 21A-21D). Colon sections (as shown in FIGS. 20A-20C) of mice described in FIGS. 19A-19C were scored for pathology (FIGS. 20A-20C). Data were combined for all animals in each cohort. Sum Colitis Scores are shown in FIG. 21A, Mean Edema Extent in FIG. 21B, Mean Histopathology Scores in FIG. 21C, and Mean Neutrophil Score in FIG. 21D. Mean Histopathology Scores include scores for mucosal thickening (hyperplasia), degree of inflammation, gland damage, and erosion extent. Mean Neutrophil Score measures neutrophil invasion which increases in inflammation.

IL10^{-/-} (Knockout) Model of Colitis.

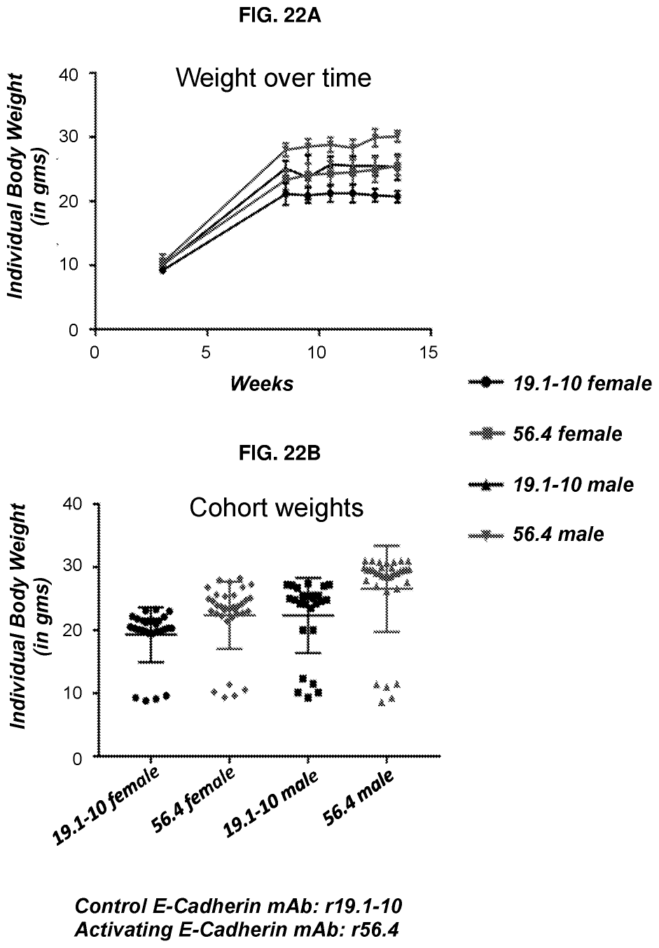

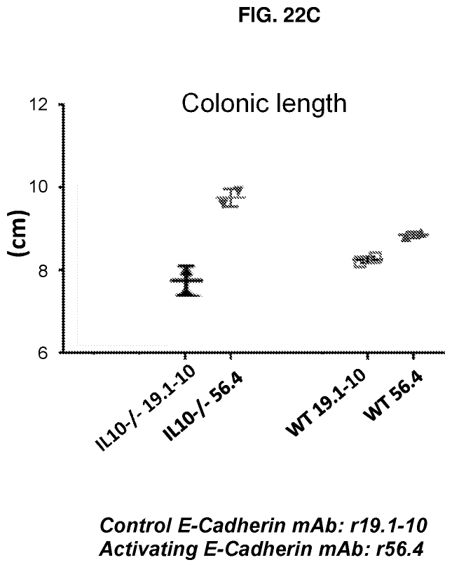

A second model employed a spontaneous genetic model of IBD, the IL-10 gene deleted (knockout, IL-10^{-/-}) mice (FIGS. 22A-24D). In this model, mice with targeted deletion of the IL-10 gene develop spontaneous pancolitis and cecal inflammation by 2-4 months of age (Berg et al. (1996).J Clin Invest 98, 1010-1020). Colons obtained from mice in this model show many of the same characteristics as those observed in human IBD by histopathological inspection. The IL-10^{-/-} model is a well-established Th1-mediated model of transmural colitis (CD4+ T cells producing Th1-type cytokines), which can be treated with various immunological agents (e.g., anti-TNF-α, anti-IFN-γ antibodies), antibiotics, and probiotics. IBD develops more slowly in this model and depends on the microbiome. Over the time course of the experiment, shown symptoms were milder than the T-cell transfer model.

Mice were treated twice weekly with IP injection of 5mg/kg of either E-cadherin activating mAb 56-4 or a control neutral mAb 19.1.10. Animals were monitored for weight loss. Colons were dissected from euthanized mice and measured. Histology of colon tissue was performed blind by HistoTox Labs (Boulder, Colo.). Standardized IBD scoring included generation of Sum Colitis Scores which includes the following parameters: Mean Edema Extent; Mean Histopathology Scores, which includes scores for mucosal thickening (hyperplasia), degree of inflammation, gland damage, and erosion extent; and Mean Neutrophil Score, which measures neutrophil invasion (score increases in inflammation).

E-Cadherin Activating mAb Reduced Overt Symptoms of Colitis. E-cadherin activating mAb treated mice did not gain as much weight as controls, nor did their intestines shrink as much (FIGS. 22A-22C). The animals did not lose weight during the short duration of this experiment, due to a slower onset of colitis and continued normal weight gain due to growth (FIG. 22A). Nonetheless, 56.4 activating mAb supported greater weight gain compared to 19.1-10 neutral mAb, potentially due to slowing of colitis induced loss. FIG. 22B shows the endpoints of body weight when the experiment was terminated. Colonic length is known to shorten in colitis. Colons were longer in activating mAb 56.4 treated mice compared to mice treated with control neutral mAb (FIG. 22C).

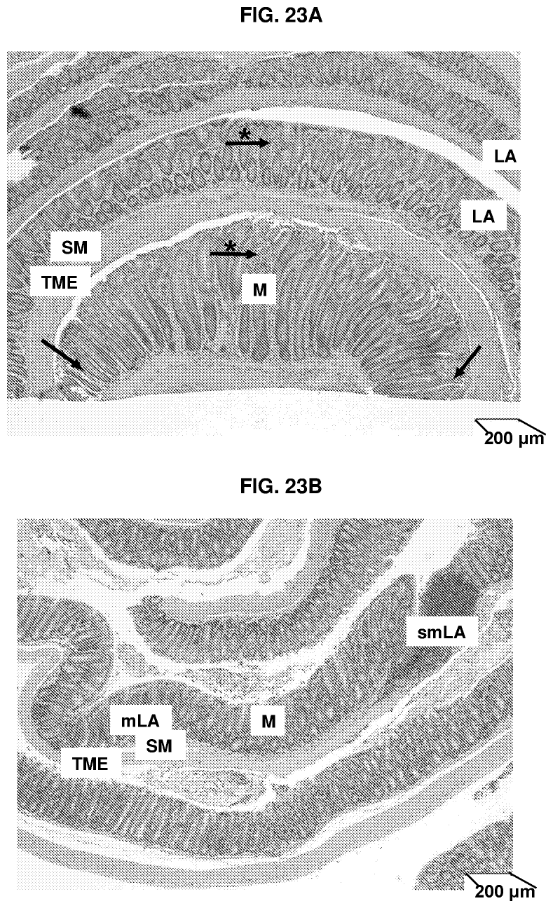

E-Cadherin Activating mAb Reduced Signs of Inflammation in Colitis. Histological examination revealed reduced signs of inflammation (FIGS. 23A, 23B). FIG. 23A shows histology of a colon from an IL-10^{-/-} mouse treated with neutral mAb 19.1-10). The lamina propria of the mucosa (M) is infiltrated by low to moderate numbers of inflammatory cells (lymphocytes, macrophages, and few neutrophils; arrows with asterisks). Inflammation mildly extends into the submucosa (SM). A region of the mucosal epithelium is hyperplastic and forms a polypoid-like structure extending into the lumen (arrows without asterisks mark the border). The remaining epithelium is mildly hyperplastic or no hyperplasia. The tunica muscularis externa (TME) and mucosal lymphoid aggregates (LA) are indicated. FIG. 23B shows histology of a colon from an IL-10^{-/-} mouse treated with activating mAb 56.4). Non-lesioned colon is captured. Mucosal glands (M), submucosa (SM), tunica muscularis externa (TME), and mucosal (mLA) and submucosal (smLA) lymphoid aggregates are indicated.

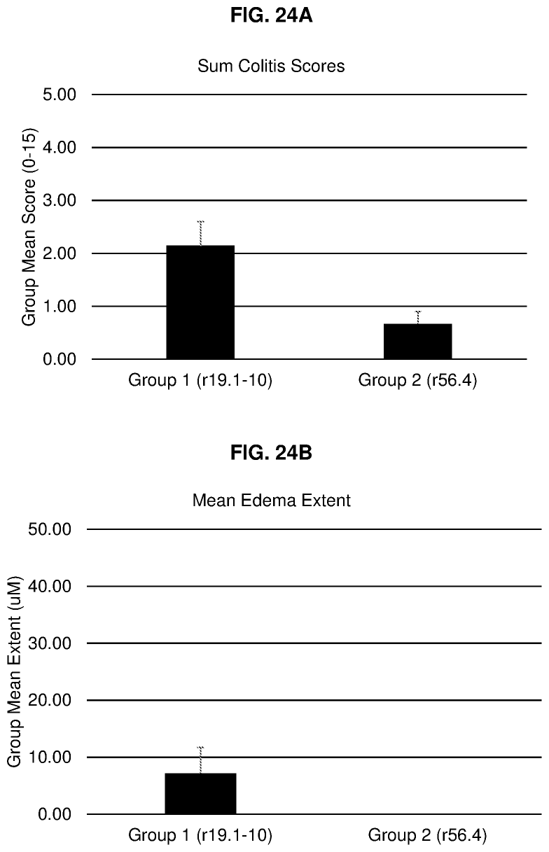

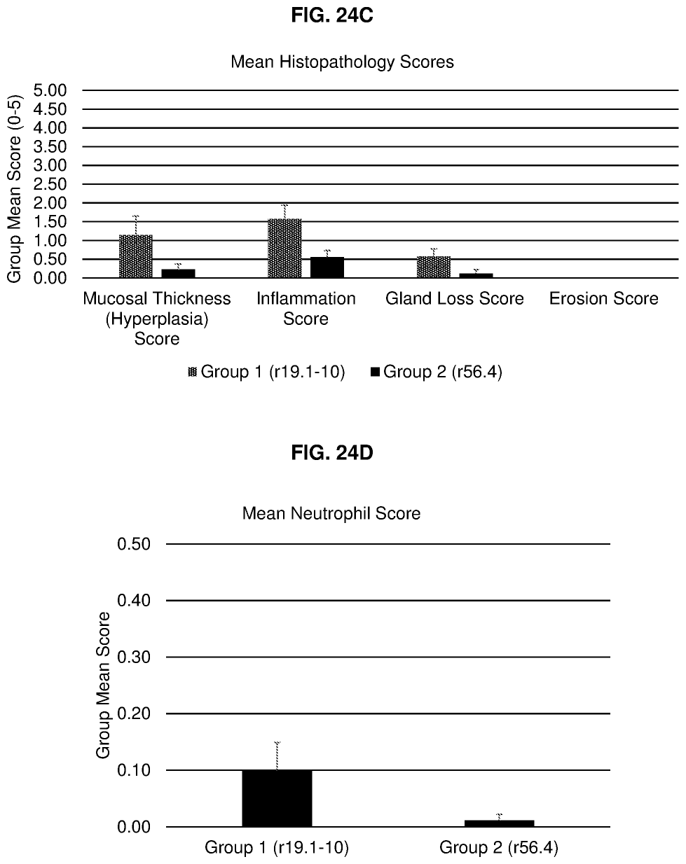

E-Cadherin Activating mAb Reduced Pathology of Colitis. Standard IBD histological analysis (carried out under contract with HistoTox, Boulder, Colo.) shows that E-cadherin activating mAb leads to a partial reduction in IBD score (FIGS. 24A-24D). Colon sections (as shown in FIGS. 23A, 23B) of mice described in FIGS. 22A-22C were scored for pathology (FIGS. 24A-24D). Data were combined for all animals in each cohort. Sum Colitis Scores are shown in FIG. 24A, Mean Edema Extent in FIG. 24B, Mean Histopathology Scores in FIG. 24C, and Mean Neutrophil Score in FIG. 24D. Mean Histopathology Scores include scores for mucosal thickening (hyperplasia), degree of inflammation, gland damage, and erosion extent. Mean Neutrophil Score measures neutrophil invasion which increases in inflammation.

### Example 5: 3D Structure of Activating Fab-E-Cadherin Complexes

E-cadherin mediated epithelial cell junctions are important for barrier function in numerous organs, especially at mucosal linings like the lung and GI tract that are sites of infection and inflammation. Barrier dysfunction has been implicated in the pathogenesis of asthma, autoimmune diseases such as Inflammatory Bowel Disease (IBD), eczema, and respiratory infections (e.g., Respiratory Syncytial Virus, coronaviruses such as SARS-CoV2). In some cases, barrier dysfunction may be an initiating event allowing pathogens, allergens, or toxins to enter and cause inflammation.

Described herein is the novel discovery that allosteric regulation of E-cadherin activity at the cell surface in response to intracellular signals controls the state of epithelial junctions. In particular, a novel class of monoclonal antibodies were generated that activate E-cadherin at the cell surface. These antibodies are being developed both as probes to understand regulation of E-cadherin, cell junctions, and barrier function, and as candidate therapeutics for treating pathophysiological disruption of epithelial barrier function in inflammation.

An important step in understanding how these activating mAbs trigger the change in cadherin function, and for developing them as potential therapeutic agents, is to determine the 3D structure of the activating Fab-E-cadherin complex. X-ray crystal structures of several E-cadherin ectodomain fragments have already been reported (Harrison et al. Structure 19(2): 244-256, 2011; Pertz et al. The EMBO journal 18(7): 1738-1747, 1999).

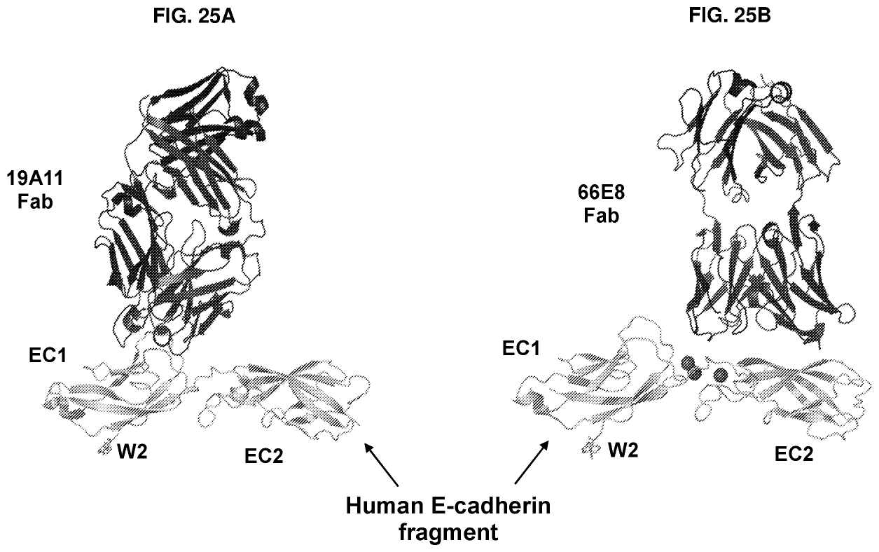

To understand the mechanism by which activating mAbs act on E-cadherin, the X-ray crystal structures of two activating Fab-E-cadherin protein fragment complexes were determined, in collaboration with colleagues at the Center for Global Infectious Disease at Seattle Children's Research Institute. Structures of E-cadherin activating Fab 19A11/human E-cadherin fragment complex and E-cadherin activating Fab 66E8/human E-cadherin fragment complex are shown in FIGS. 25A and 25B, respectively. Subtle changes in the structure of the E-cadherin fragment have been observed. There are small changes in sites where the Fabs bind and a small shift in the position of the key Trp2 residue involved in forming the adhesive bond. The Fabs may also interfere with a putative X-dimer intermediate.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect, in this context, is a measurable change in the ability of an anti-E-cadherin antibody to activate adhesion activity, or to influence cancer metastasis or an inflammatory disease or condition involving disruption in normal cell adhesion or cell junctions.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. An engineered antibody comprising:
the heavy chain CDR1, CDR2 and CDR3 shown, respectively, in SEQ ID NO: 17, 18, and 19, and the light chain CDR1, CDR2 and CDR3 shown, respectively, in SEQ ID NO: 20, 21, and 22; or
the heavy chain CDR1, CDR2 and CDR3 shown, respectively, in SEQ ID NO: 23, 24, and 25, and the light chain CDR1, CDR2 and CDR3 shown, respectively, in SEQ ID NO: 26, 27, and 28; or
the heavy chain CDR1, CDR2 and CDR3 shown, respectively, in SEQ ID NO: 29, 30, and 31, and the light chain CDR1, CDR2 and CDR3 shown, respectively, in SEQ ID NO: 32, 33, and 34; or
the heavy chain CDR1, CDR2 and CDR3 shown, respectively, in SEQ ID NO: 35, 36, and 37, and the light chain CDR1, CDR2 and CDR3 shown, respectively, in SEQ ID NO: 38, 39, and

40.

2. The engineered antibody of claim 1, which is a humanized antibody.

3. The engineered antibody of claim 1, which is a Fab, an IgG, a scFv, a diabody, or bispecific antibody.

4. The engineered antibody of claim 1, which binds specifically to and activates E-cadherin.

5. An engineered antibody that binds specifically to and activates E-cadherin, comprising:
the heavy chain variable (VH) domain shown in SEQ ID NO: 2 and the light chain variable (VL) domain shown in SEQ ID NO: 4; or
the VH domain shown in SEQ ID NO: 6 and the VL domain having SEQ ID NO: 8; or
the VH domain shown in SEQ ID NO: 10 and the VL domain shown in SEQ ID NO: 12; or
the VH domain shown in SEQ ID NO: 14 and the VL domain shown in SEQ ID NO: 16.

6. The engineered antibody of claim 5, comprising:
the VH domain shown in SEQ ID NO: 2 and the VL domain shown in SEQ ID NO: 4.

7. The engineered antibody of claim 5, comprising:
the VH domain shown in SEQ ID NO: 6 and the VL domain having SEQ ID NO: 8.

8. The engineered antibody of claim 5, comprising:
the VH domain shown in SEQ ID NO: 10 and the VL domain shown in SEQ ID NO: 12.

9. The engineered antibody of claim 5, comprising:
the VH domain shown in SEQ ID NO: 14 and the VL domain shown in SEQ ID NO: 16.

10. An engineered antibody comprising the monoclonal antibody 19A11, 66E8, 56-4, or 18-5, or a humanized version or functional fragment thereof.

11. A polynucleotide encoding the antibody of claim 5, wherein the polynucleotide comprises:
the polynucleotide sequence encoding the VH domain shown in SEQ ID NO: 1; or the polynucleotide sequence encoding the VL domain that is shown in SEQ ID NO: 3; or both.

12. A polynucleotide encoding the antibody of claim 5, wherein the polynucleotide comprises:
the polynucleotide sequence encoding the VH domain shown in SEQ ID NO: 5; or
the polynucleotide sequence encoding the VL domain that is shown in SEQ ID NO: 7; or both.

13. A polynucleotide encoding the antibody of claim 5, wherein the polynucleotide comprises:
the polynucleotide sequence encoding the VH domain shown in SEQ ID NO: 9; or
the polynucleotide sequence encoding the VL domain that is shown in SEQ ID NO: 11; or both.

14. A polynucleotide encoding the antibody of claim 5, wherein the polynucleotide comprises:
the polynucleotide sequence encoding the VH domain shown in SEQ ID NO: 13; or
the polynucleotide sequence encoding the VL domain that is shown in SEQ ID NO: 15; or both.

15. Use of the antibody of any one of claims 1-10 or encoded by the polynucleotide of any one of claims 11-14 that specifically binds to and activates human E-cadherin to treat, prevent, or ameliorate:
cancer metastasis;
inflammatory bowel disease; or
airway inflammation.

16. The use of claim 15, wherein the airway inflammation comprises acute respiratory distress syndrome (ARDS).

17. A method for treating cancer in a subject, comprising:
administering to a subject in need of such treatment a therapeutically effective amount of the engineered antibody of any one of claims 1-10 or encoded by the polynucleotide of any one of claims 11-14 that specifically binds to and activates human E-cadherin.

18. The method of claim 17, wherein treating cancer comprises reducing cancer metastasis.

19. A method of treating a cancer patient with a cancer that expresses an E-cadherin protein, comprising:
obtaining a tissue sample from an individual at risk of having a cancer that expresses an E-cadherin protein;
determining the presence or absence or amount of the E-cadherin protein in the tissue sample in comparison to a control tissue sample from an individual known to be negative for the cancer; thereby diagnosing the individual at risk as a cancer patient with a cancer that expresses an E-cadherin protein, wherein the E-cadherin protein is expressed at normal or low levels, or is expressed by a subset of cells, or is overexpressed; and
administering to the cancer patient with a cancer that expresses an E-cadherin protein a therapeutically effective amount of the engineered antibody of any one of claims 1-10 or encoded by the polynucleotide of any of claims 11-14, or an antigen-binding antibody fragment thereof, that specifically binds to and activates human E-cadherin.

20. A method for treating a subject having an inflammatory disorder comprising:
administering to a subject in need of such treatment a therapeutically effective amount of the engineered antibody of any one of claims 1-10 or encoded by the polynucleotide of any one of claims 11-14 that specifically binds to and activates human E-cadherin.

21. The method of claim 20, wherein the inflammatory disorder comprises inflammatory bowel disease or airway inflammation.

22. The method of claim 21, wherein the airway inflammation comprises acute respiratory distress syndrome (ARDS).

23. The method of claim 20, wherein the inflammatory disorder comprises an autoimmune disease.

24. The method of claim 20, wherein the inflammatory disorder is characterized by disruption of normal cell adhesion and/or cell junctions.

25. The method of claim 20, wherein the engineered antibody is administered locally to a site of inflammation in the subject.

26. The method of claim 20, wherein the engineered antibody comprises monoclonal antibody 19A11, 66E8, or a humanized version or functional fragment thereof.

27. A method for modulating cell adhesion of E-cadherin-expressing cells comprising:
contacting the cells with the engineered antibody of any one of claims 1-10 or the engineered antibody encoded by the polynucleotide of any of claims 11-14.


SEQUENCE LISTING (64 sequences)

SEQ ID NO: 1 - DNA, 414 bp, Mus musculus
atggctgtcctggggctgcttctctgcctggtgacgttcccaagctgtgtcctgtcccaggtgcagctgaaggagtcaggacctggcctggtggcaccctcacagagcctgtccatcacatgcacggtctctgggttctcattatccagatatggtgtacactgggttcgccagcctccaggaaagggtctggagtggctgggaatgatgtggggtggtggaaacacagactataattcagctctcaaatccagactgagcatcagcaaggacaactccaagagccaagttttcttaaaaatgaacagtctgcaaactgatgacacagccatgtactactgtgccagtagtaactacgttcttgggtatgctatggactactggggtcaaggaacctcagtcaccgtctcctca

SEQ ID NO: 2 - protein, 138 aa, Mus Musculus
MAVLGLLLCLVTFPSCVLSQVQLKESGPGLVAPSQSLSITCTVSGFSLSRYGVHWVRQPPGKGLEWLGMMWGGGNTDYNSALKSRLSISKDNSKSQVFLKMNSLQTDDTAMYYCASSNYVLGYAMDYWGQGTSVTVSS

SEQ ID NO: 3 - DNA, 399 bp, Mus musculus
atggaatcacagacccaggtcctcatgtttcttctgctctgggtatctggtgcctgtgcagacattgtgatgacacagtctccatcctccctggctatgtcagtaggacagaaggtcactatgaactgcaagtccagtcagagtcttttaaatagtagcaatcaaaagaactatttggcctggtaccagcagaaaccaggacagtctcctaaacttctgatatactttacatccactaggggatctggggtccctgatcgcttcataggcagtggatctgggacagatttcactcttaccatcagcagtgtggaggctgaagacctggcagattacttctgtcagcaacattatagaactccgcacacgttcggaggggggaccaaggtggaaataaaa

SEQ ID NO: 4 - protein, 133 aa, Mus musculus
MESQTQVLMFLLLWVSGACADIVMTQSPSSLAMSVGQKVTMNCKSSQSLLNSSNQKNYLAWYQQKPGQSPKLLIYFTSTRGSGVPDRFIGSGSGTDFTLTISSVEAEDLADYFCQQHYRTPHTFGGGTKVEIK

SEQ ID NO: 5 - DNA, 405 bp, Mus musculus
atgggatggagctgtgtctttctctttctcctgtcagtaactgtaggtgtgttctctgaggttcagctgcagcagtctggacctgagctggtgaagcctggggcttcagtgaagatatcctgcaaggcttcaggttactcatttactggctactttatgaactgggtgaagcagagccatggaaagagccttgagtggattggacgtattaatccttacaatggtgatactttctacaagcagaggttcaagggcaaggccacattgactgtagacaaatcctctagcacagtccacatggacctcctgagcctgacatctgaggactctgcagtctattattgtggaagaggtaactactactttgactactggggccaaggcaccactctcacagtctcctca

SEQ ID NO: 6 - protein, 135 aa, Mus musculus
MGWSCVFLFLLSVTVGVFSEVQLQQSGPELVKPGASVKISCKASGYSFTGYFMNWVKQSHGKSLEWIGRINPYNGDTFYKQRFKGKATLTVDKSSSTVHMDLLSLTSEDSAVYYCGRGNYYFDYWGQGTTLTVSS

SEQ ID NO: 7 - DNA, 381 bp, Mus musculus
atgaggttccaggttcaggttctggggctccttctgctctggatatcaggtgcccagtgtgatgtccagataacccagtctccatcttatcttgctgcatctcctggagaaaccattactattaattgcaggacaagtaagaacattagcaagtatttagcctggtatcaagagaaacctgggaaaactaataagcttcttatctactctggatacactttgcagtctggaattccatcaaggttcagtggcagtggatctggtacagatttcactctcaccatcagtagcctggagcctgaagattttgcaatgtattactgtcaacagcataatgaatacccgtacacgttcggaggggggaccaagctggaaattaaa

SEQ ID NO: 8 - protein, 127 aa, Mus musculus
MRFQVQVLGLLLLWISGAQCDVQITQSPSYLAASPGETITINCRTSKNISKYLAWYQEKPGKTNKLLIYSGYTLQSGIPSRFSGSGSGTDFTLTISSLEPEDFAMYYCQQHNEYPYTFGGGTKLEIK

SEQ ID NO: 9 - DNA, 426 bp, Oryctolagus cuniculus
atggagactgggctgcgctggcttctcctggtcgctgtgctcaaaggtgtccagtgtcagcaggagctggaggagtccgggggaggcctggtcaggcctggggcatccctgacactcacctgcaaagcctctggattcgacctcagtaactactactacttgtgctgggtccgccagtctccagggaaggggctggagtggatcgcatgcatttatgctggtgctactcatgacacttactacgcgaactgggcgaaaggccgattcaccatctccaggacctcgtcgaccacggtgactctgcaaatgaccagtctgacagacgcggacacggccacctatttctgtgcgagagacatttttgctagtggtacttattattatcgggccttgtggggcccgggcaccctggtcaccgtctcctca

SEQ ID NO: 10 - protein, 142 aa, Oryctolagus cuniculus
METGLRWLLLVAVLKGVQCQQELEESGGGLVRPGASLTLTCKASGFDLSNYYYLCWVRQSPGKGLEWIACIYAGATHDTYYANWAKGRFTISRTSSTTVTLQMTSLTDADTATYFCARDIFASGTYYYRALWGPGTLVTVSS

SEQ ID NO: 11 - DNA, 390 bp, Oryctolagus cuniculus
atggacatgagggcccccactcagctgctggggctcctgctgctctggctcccaggtgccagatgtgcattcgaattgacccagactccatcctccgtggaggcagctgtgggaggcacagtcaccatcaattgccaggccagtcagagcattaatagttggttatcctggtatcagcagaaaccagggcagcctcccaagctcctgatctatgctgcatccactctggcatctggagtctcatcgcggttcaaaggcagtggatctgggacagagttcgctctcaccatcagcgacctggagtgtgccgatgctgccacttactactgtcagagctattatggaactagtactactgatttcggcggagggaccgaggtggtggtcaaa

SEQ ID NO: 12 - protein, 130 aa, Oryctolagus cuniculus
MDMRAPTQLLGLLLLWLPGARCAFELTQTPSSVEAAVGGTVTINCQASQSINSWLSWYQQKPGQPPKLLIYAASTLASGVSSRFKGSGSGTEFALTISDLECADAATYYCQSYYGTSTTDFGGGTEVVVK

SEQ ID NO: 13 - DNA, 426 bp, Oryctolagus cuniculus
atggagactgggctgcgctggcttctcctggtcgctgtgctcaaaggtgtccagtgtcaggagcagctggaggagtccgggggaggcctggtcaagcctggggcatccctgacactcacctgcacagcctctggattcgacctcagtacctatttctacatgtgctgggtccgccaggctccagggaaggggctggagtggatcgcatgcatttatgttggtagtactggtgacacttactacgcgaactgggcgaaaggccgattcaccatctccaaaacctcgtcgaccacggtgactctgcaaatgaccagtctgacagacgcggacacggccacctatttctgtgcgagagacatttttgctactggtattaattattatcgggccttgtggggcccaggcaccctggtcaccgtctcctca

SEQ ID NO: 14 - protein, 142 aa, Oryctolagus cuniculus
METGLRWLLLVAVLKGVQCQEQLEESGGGLVKPGASLTLTCTASGFDLSTYFYMCWVRQAPGKGLEWIACIYVGSTGDTYYANWAKGRFTISKTSSTTVTLQMTSLTDADTATYFCARDIFATGINYYRALWGPGTLVTVSS

SEQ ID NO: 15 - DNA, 390 bp, Oryctolagus cuniculus
atggacatgagggcccccactcagctgctggggctcctgctgctctggctcccaggtgccagatgtgcattcgaattgacccagactccagcctccgtggaggcaggtgtgggaggcacagtcaccatcaagtgccaggccagtgagagcattaatagttggttatcctggtatcagcagaaaccagggcagcctcccacgctcctgatctattctgcatccactctggcatctggggtcccatcgcgtttcaaaggcagtagatctgggacacagttcactctcaccatcagcgacctggagtgtgccgatgctgccacttactactgtcaaagctattatggaactagtactactgctttcggcggagggaccgaggtggtggtcaaa

SEQ ID NO: 16 - protein, 130 aa, Oryctolagus cuniculus
MDMRAPTQLLGLLLLWLPGARCAFELTQTPASVEAGVGGTVTIKCQASESINSWLSWYQQKPGQPPTLLIYSASTLASGVPSRFKGSRSGTQFTLTISDLECADAATYYCQSYYGTSTTAFGGGTEVVVK

SEQ ID NO: 17 - protein, 5 aa, Mus musculus
RYGVH

SEQ ID NO: 18 - protein, 16 aa, Mus musculus
MMWGGGNTDYNSALKS

SEQ ID NO: 19 - protein, 11 aa, Mus musculus
SNYVLGYAMDY

SEQ ID NO: 20 - protein, 17 aa, Mus musculus
KSSQSLLNSSNQKNYLA

SEQ ID NO: 21 - protein, 7 aa, Mus musculus
FTSTRGS

SEQ ID NO: 22 - protein, 9 aa, Mus musculus
QQHYRTPHT

SEQ ID NO: 23 - protein, 5 aa, Mus musculus
GYFMN

SEQ ID NO: 24 - protein, 17 aa, Mus musculus
RINPYNGDTFYKQRFKG

SEQ ID NO: 25 - protein, 7 aa, Mus musculus
GNYYFDY

SEQ ID NO: 26 - protein, 11 aa, Mus musculus
RTSKNISKYLA

SEQ ID NO: 27 - protein, 7 aa, Mus musculus
SGYTLQS

SEQ ID NO: 28 - protein, 9 aa, Mus musculus
QQHNEYPYT

SEQ ID NO: 29 - protein, 6 aa, Oryctolagus cuniculus
NYYYLC

SEQ ID NO: 30 - protein, 18 aa, Oryctolagus cuniculus
CIYAGATHDTYYANWAKG

SEQ ID NO: 31 - protein, 13 aa, Oryctolagus cuniculus
DIFASGTYYYRAL

SEQ ID NO: 32 - protein, 11 aa, Oryctolagus cuniculus
QASQSINSWLS

SEQ ID NO: 33 - protein, 7 aa, Oryctolagus cuniculus
AASTLAS

SEQ ID NO: 34 - protein, 10 aa, Oryctolagus cuniculus
QSYYGTSTTD

SEQ ID NO: 35 - protein, 6 aa, Oryctolagus cuniculus
TYFYMC

SEQ ID NO: 36 - protein, 18 aa, Oryctolagus cuniculus
CIYVGSTGDTYYANWAKG

SEQ ID NO: 37 - protein, 13 aa, Oryctolagus cuniculus
DIFATGINYYRAL

SEQ ID NO: 38 - protein, 11 aa, Oryctolagus cuniculus
QASESINSWLS

SEQ ID NO: 39 - protein, 7 aa, Oryctolagus cuniculus
SASTLAS

SEQ ID NO: 40 - protein, 10 aa, Oryctolagus cuniculus
QSYYGTSTTA

SEQ ID NO: 41 - DNA, 28 bp, Artificial Sequence
  Synthetic oligonucleotide
cccgccttatgattctctgctcgtgtcc

SEQ ID NO: 42 - DNA, 27 bp, Artificial Sequence
  Synthetic oligonucleotide
tccgtacatgtcagccagcttcttgaa

SEQ ID NO: 43 - DNA, 21 bp, Artificial Sequence
  Synthetic oligonucleotide
tccttgtctacgctttccacg

SEQ ID NO: 44 - DNA, 18 bp, Artificial Sequence
  Synthetic oligonucleotide
ggtcgcattgtggccttt

SEQ ID NO: 45 - DNA, 21 bp, Artificial Sequence
  Synthetic oligonucleotide
cccgagaggtctttttccgag

SEQ ID NO: 46 - DNA, 21 bp, Artificial Sequence
  Synthetic oligonucleotide
ccagcccatgatggttctgat

SEQ ID NO: 47 - DNA, 22 bp, Artificial Sequence
  Synthetic oligonucleotide
tgaccctgatgagaaagctcaa

SEQ ID NO: 48 - DNA, 21 bp, Artificial Sequence
  Synthetic oligonucleotide
ccctgagcaacactgtctttt

SEQ ID NO: 49 - DNA, 19 bp, Artificial Sequence
  Synthetic oligonucleotide
cgaccctgcctctgaatcc

SEQ ID NO: 50 - DNA, 21 bp, Artificial Sequence
  Synthetic oligonucleotide
tacacgctgggaaacatgagc

SEQ ID NO: 51 - DNA, 25 bp, Artificial Sequence
  Synthetic oligonucleotide
agaatactgtcatttgctccaaact

SEQ ID NO: 52 - DNA, 18 bp, Artificial Sequence
  Synthetic oligonucleotide
gttctggatcgccccttc

SEQ ID NO: 53 - DNA, 20 bp, Artificial Sequence
  Synthetic oligonucleotide
taccggagagcgttcagtga

SEQ ID NO: 54 - DNA, 22 bp, Artificial Sequence
  Synthetic oligonucleotide
ccatcccgaaagagttcattca

SEQ ID NO: 55 - DNA, 20 bp, Artificial Sequence
  Synthetic oligonucleotide
ttgctacagggtttcatcca

SEQ ID NO: 56 - DNA, 23 bp, Artificial Sequence
  Synthetic oligonucleotide
catattgctgtccagttcatctc

SEQ ID NO: 57 - DNA, 20 bp, Artificial Sequence
  Synthetic oligonucleotide
gctcagcaaggaggtaggtg

SEQ ID NO: 58 - DNA, 20 bp, Artificial Sequence
  Synthetic oligonucleotide
tcttaccggtgtccaagtcc

SEQ ID NO: 59 - DNA, 26 bp, Artificial Sequence
  Synthetic oligonucleotide
ctccaacagatacacccgcacatact

SEQ ID NO: 60 - DNA, 26 bp, Artificial Sequence
  Synthetic oligonucleotide
gtatccagaaagcgaccaagaccagc

SEQ ID NO: 61 - DNA, 972 bp, Mus musculus
gggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 62 - protein, 323 aa, Mus musculus
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 63 - DNA, 315 bp, Mus musculus
ggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 64 - protein, 104 aa, Mus musculus
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC